(19) United States
(12) Patent Application Publication
(10) Pub. No.: US 2021/0401220 A1
(43) Pub. Date: Dec. 30, 2021

(54) DEVICE FOR METERING AND/OR FOR PREPARING A MEDIUM TO BE PREPARED, CONTAINER FOR RECEIVING AND METERING A COMPONENT, CONTAINER FOR RECEIVING AND METERING FLUID, AND CORRESPONDING SYSTEM

(71) Applicant: SMIICS GmbH, Göttingen (DE)

(72) Inventor: Stefan SCHLACK, Göttingen (DE)

(21) Appl. No.: 17/293,778

(22) PCT Filed: Dec. 5, 2019
(86) PCT No.: PCT/EP2019/083775

§ 371 Date: May 13, 2021

Related U.S. Application Data
(63) Continuation-in-part of application No. 16/070,084, filed on Jul. 13, 2018, now Pat. No. 11,033,141.

(30) Foreign Application Priority Data
Jan. 14, 2016 (DE) 10 2016 000 406.1
Dec. 5, 2018 (DE) 10 2018 009 551.8
Feb. 13, 2019 (DE) 10 2019 001 063.9
Jul. 17, 2019 (DE) 10 2019 005 000.2
Oct. 21, 2019 (DE) 20 2019 004 331.4

(51) Int. Cl. A47J 31/40; B65D 83/06; A47J 31/54; A47J 31/46; A47J 31/42; A47J 31/52; A47J 31/44; A47J 31/41

(52) U.S. Cl. CPC A47J 31/404; B65D 83/06; A47J 31/54; A47J 31/462; A47J 31/42; A47J 2203/00; A47J 31/4403; A47J 31/525; A47J 31/402; A47J 31/41; A47J 31/521

(57) ABSTRACT

A device (1; 1′) is shown and described for metering and/or preparing a medium to be prepared, in particular baby food, in particular baby milk formula or semi-solid baby food, coffee and/or tea, the device comprising a housing (3) with a first receiving region (5; 5′) and a second receiving region (7; 7′), wherein the first receiving region (5; 5′) is configured for receiving a first container (9; 9′) for a first component of the medium to be prepared, and wherein the second receiving region (7; 7′) is configured for receiving a second container (11) for a fluid, a temperature control device for controlling the temperature of the fluid, a metering device (29; 29′) for metering the first component, wherein the first receiving region (5; 5′) has a metering device receiving region (27; 27′) for receiving the metering device (29; 29′), and wherein an actuating and/or drive device (39) for the metering device (29; 29′) is arranged in the metering device receiving region (27; 27′).

The present application relates to a device for dosing and/or preparing a medium to be prepared, in particular baby food, in particular baby milk or baby food, coffee and/or tea, a container for receiving and dosing a component for preparing a medium, in particular Baby milk or baby food, coffee and/or tea, a container for receiving and dosing fluid for preparing a medium to be prepared, in particular baby food, in particular baby milk or baby food, coffee and/or tea, and a system comprising a device for dosing and/or preparing a medium to be prepared, a container for receiving and dosing a component for preparing a medium, and a container for receiving and dosing fluid for preparing a medium to be prepared.

Exact and precise dosing plays an important role in the preparation of a large number of media, especially in media in which a fluid, for example a liquid or water, has to be mixed with a component, for example a powder or a concentrate. For example, when preparing baby food from baby food concentrate, a corresponding powder or amount of concentrate is measured out or dosed and mixed with water before the baby food is administered. Or when preparing coffee, for example, a corresponding amount of coffee powder or coffee beans must be measured or dosed and mixed with the desired amount of water.

Starting from the prior art, the object of the present invention is to enable a simplified dosage and/or preparation of a medium to be prepared, in particular baby food, in particular baby milk or baby food, coffee and/or tea.

According to the invention, this object is achieved by the subject matter of the independent claims. Preferred embodiments emerge from the dependent claims.

According to one aspect of the invention, a device for dosing and/or preparing a medium to be prepared, in particular baby food, in particular baby milk or baby food, coffee and/or tea, the device comprising: a housing with a first receiving area and a second receiving area, wherein the first receiving area is designed to receive a first container for a first component of the medium to be prepared and wherein the second receiving area is designed to receive a second container for a fluid, a tempering device for tempering the fluid, a dosing device for dosing the first component, wherein the first receiving area has a dosing device receiving area for receiving the dosing device, and wherein an actuating and/or drive device for the dosing device is arranged in the dosing device receiving area.

Preferably, the second container can be connected to a fluid reservoir, the second container and/or the fluid reservoir being exchangeable and designed as disposable article.

Preferably, the dosing device is connected to the first container, the first container and the dosing device being interchangeable and designed as disposable article.

According to a further aspect of the invention, a container for receiving and dosing a component for the preparation of a medium, in particular baby food, in particular baby milk or baby food, coffee and/or tea, the container comprising: a housing with an interior space for receiving the component; and an outlet in fluid communication with the interior space, the outlet being connectable to an inlet of a dosing device, the dosing device having an outlet such that actuation of the dosing device dispenses a dosage of the component through the outlet; wherein the dosing device is or can be connected to the container, and wherein the container and/or the dosing device are exchangeable and are designed as disposable articles.

Preferably, the container for receiving and dosing a component for preparing a medium, in particular baby food, in particular baby milk or baby food, coffee and/or tea, can be supplied pre-filled with the component.

According to a further aspect of the invention, a container for receiving and dosing fluid for the preparation of a medium to be prepared, in particular baby food, in particular baby milk or baby food, coffee and/or tea, the container comprising: a housing with an interior space for receiving of fluid, an inlet in fluid connection with the interior space and an outlet in fluid connection with the interior space, the inlet being connectable to an outlet of a fluid reservoir, wherein a dosage of the fluid for preparing the medium to be prepared can be delivered through the outlet of the container, and wherein the container is replaceable and designed as a disposable article.

Preferably, the container for receiving and dosing fluid for the preparation of a medium to be prepared, in particular baby food, in particular baby milk or baby food, coffee and/or tea can be supplied pre-filled with the fluid.

Another aspect of the invention relates to a system comprising a device for preparing a medium to be prepared, in particular baby food, in particular baby milk or baby food, coffee and/or tea, a first container for receiving and dosing a component for preparing a medium, and/or a second container for receiving and dosing fluid for preparing a medium to be prepared, in particular baby food, in particular baby milk or baby food, coffee and/or tea.

In the following, the invention is first described on the basis of a device for dosing and grinding coffee beans and/or for preparing coffee, in particular filter coffee. Thereafter, the invention is described on the basis of a device for dosing and/or preparing baby food, in particular baby milk or baby food, or coffee. The description is thus based on coffee and baby food as media to be prepared. It is conceivable that the medium to be prepared can also be any other medium, for example tea, soup, or the like.

There are various known ways of preparing filter coffee. For example, the filter coffee can be prepared in a Chemex carafe using a special Chemex filter, or in a Karlsbader jug, where the coffee is filtered through a fine, double-glazed porcelain sieve. Furthermore, so-called dripping coffee machines are on the market, in which cold water gradually seeps through a paper filter, drop by drop, onto the coffee powder and collects as iced coffee in a glass jug below. Depending on the type of preparation, different filters, grinding grades, temperatures, mixing ratios, coffee soaking times, water dispensing speeds, etc. are used.

In order for the filter coffee to develop a particularly good aroma, the coffee should ideally be freshly ground, as the aroma disappears with it. So far, coffee grinders for filter coffee are used for this purpose. In the manual process, the ground coffee is then weighed and, depending on the desired coffee volume and type of coffee, a certain mixing ratio must be maintained between the ground coffee and a fluid, for example water, so that the coffee develops a particularly aromatic taste.

Filter coffee machines are known which include such a coffee grinder. These coffee machines known from the prior art can grind the coffee beans and heat the coffee that is then brewed, but these coffee machines must be cleaned regularly. Coffee residues, for example oil residues, etc., can worsen the coffee taste and germs or bacterially contaminated residues can occur. Furthermore, the machines can calcify over time and not only be irreparably damaged as a result, because the calcification in turn has a detrimental effect on the aroma of the coffee. Therefore, all coffee machines known from the prior art with an integrated grinder must be cleaned and decalcified regularly. The lines by means of which the fluid for preparing coffee is supplied must also be cleaned in this context, for example to avoid a bio-film.

It is desirable to provide a device for dosing and grinding coffee beans and/or for preparing coffee, by means of which the preparation of coffee with an improved aroma is made possible in a simple manner.

Preferably, a device for dosing and grinding coffee beans and/or for preparing coffee, in particular filter coffee, is provided, comprising: a housing with a first receiving area and a second receiving area, the first receiving area being designed to receive a first container for coffee beans and wherein the second receiving area is designed to receive a second container for a fluid, a tempering device for controlling the temperature of the fluid, a dosing and grinding device for dosing and grinding the coffee beans, the first receiving area being a dosing and grinding device receiving area for receiving the dosing and grinding device having grinding device, and wherein an actuating and/or drive device for the dosing and grinding device is arranged in the dosing and grinding device receiving area.

The device according to the invention is designed to dose and grind coffee beans and/or to prepare coffee, in particular filter coffee.

The device can have a first receiving area which is designed to receive a first container with coffee beans. Furthermore, a dosing and grinding device receiving area is arranged in the first receiving area of the device, that is to say a receiving area in which a dosing and grinding device for dosing and grinding coffee beans can be received. Thus, the first container with coffee beans and the dosing and grinding device can be at least partially received in the first receiving area. This advantageously enables the dosing and grinding device to interact with the first container. In particular, correct dosing of the coffee beans can be carried out by the dosing and grinding device. This is made possible by the fact that the dosing and grinding device is driven by the drive device which is also arranged in the dosing and grinding device receiving area.

Furthermore, all components of the device described which come into contact with the coffee beans or the coffee powder ground by the dosing and grinding device or with the fluid are in particular exchangeable and can be easily removed from the device. Exchangeable components mean that the components are designed as disposable or single-use-article. In particular, the first container for coffee beans, the dosing and grinding device for dosing and grinding the coffee beans, and the second container for a fluid are exchangeable. The first container can be connected or fluidly connected to the dosing and grinding device and/or the second container can be connected or fluidly connected to a fluid reservoir. This is advantageous because the device for dosing and grinding coffee beans and/or for preparing coffee, in particular the first receiving area and the second receiving area, does not come into contact with the coffee beans and the fluid. The device, in particular the first receiving area and the second receiving area, is not contaminated with coffee or fluid, so that cleaning of the device is not necessary after each individual preparation of coffee. Furthermore, the device and/or its individual components do not need to be decalcified.

The coffee beans are first dosed by the dosing and grinding device and then ground into coffee powder in the dosed amount. The coffee powder and the supplied fluid can then be introduced into a container, preferably into a filter or filter container, in the correct mixing ratio. This is advantageous for the desired aroma of the coffee to be prepared.

It is conceivable that the device comprises a preparation device for preparing coffee from the coffee powder and the fluid ground by the dosing and grinding device, which can also be exchangeable and designed as a disposable or single-use-articles. With the device, the ground coffee and the fluid (e.g. a liquid) can be fed from the second container in a correct mixing ratio to the preparation device, so that the ground coffee and the fluid can be fed into a further, separate container, in particular into a filter and/or funnel containers are introduced. This enables the coffee, in particular the filter coffee, to be prepared correctly, which has an advantageous effect on the quality of the aroma of the coffee.

The preparation device can thus have a filter and/or funnel container or filter container into which the coffee powder and the fluid can be introduced and/or mixed. Furthermore, the preparation device can have a container, for example a coffee cup or a coffee pot, or the preparation device can be brought into contact or interact with a container, for example a coffee cup or a coffee pot. The coffee cup or the coffee pot is arranged relative to the filter and/or funnel container in such a way that the coffee can be introduced or filled in from the filter and/or funnel container as a result of gravity. The coffee cup or the coffee pot is preferably arranged below the filter and/or funnel container.

Preferably, the device is designed to determine the presence and/or the type of preparation device.

The temperature of the fluid in the second container can be brought to the preparation temperature suitable for the production of filter coffee by means of the tempering device. For the preparation of the filter coffee, the preferred preparation temperature is between 90° C. and 100° C., particularly preferably around 96° C. This temperature should be kept as constant as possible, which is made possible by the tempering device. The tempering device can be designed as a heating plate, by means of which the fluid in the second container can be brought to a constant temperature. In contrast to a flow heater, this is possible. The tempering device can heat or heat the fluid in the interior of the second container as a whole, similar to what is the case with a high-speed water quick boiler. Thus, with the tempering device designed as a heating plate, a similar effect can be achieved as when pouring with a water quick boiler, which enables particularly good coffee to be prepared. In particular, a swelling or bloom of the coffee powder brought into contact with the fluid from the second container is made possible.

Preferably, the tempering device is controllable or regulatable. For this purpose, the device can comprise a control or regulating unit. It is conceivable that the tempering device is designed as a heating plate and/or a cooling plate or that the tempering device comprises at least one heating plate and/or at least one cooling plate. It is also conceivable that different zones or areas of the tempering device or the heating plate and/or the cooling plate can be controlled or regulated or activated.

The device for dosing and grinding coffee beans and/or for preparing coffee enables automatic dosing of fluid in the desired amount with which the coffee powder ground by the dosing and grinding device is mixed, whereby the desired aroma of the coffee is automatically obtained. This means that the fluid with which the ground coffee powder is mixed does not have to be precisely measured and matched to the amount of coffee powder, as is the case with conventional coffee filter machines, in which the complete fluid that is in the fluid tank of the coffee filter machine is available stands, is consumed. An automated comparison between fluid and coffee beans and/or ground coffee powder prevents incorrect settings of the correct mixing ratio between fluid and coffee powder, which has an advantageous effect on the aroma of the coffee.

The device for dosing and grinding coffee beans and/or for preparing coffee enables, due to the interchangeable components, that different first containers with different types of coffee beans can be introduced into the first receiving area, so that different types of coffee can be prepared with the device. Advantageously, the first container designed as a disposable article or single-use-articles and the dosing and grinding device designed as a disposable article or single-use-articles avoid residues in the individual components of the device from previous types of coffee. This means that cleaning can be dispensed with and the aroma of a newly prepared coffee is not impaired by the residues of the previously prepared coffee. An infinite number of different types of coffee could be prepared or processed without residue or without cleaning the device for dosing and grinding coffee beans and/or for preparing coffee.

Preferably, the dosing and grinding device can be connected to the first container.

The dosing and grinding device can be connectable to the first container. This means that the dosing and grinding device can be connected to the first container, so that the dosing and grinding device and the first container can be introduced into the receiving area and/or removed again together. For example, the dosing and grinding device can be firmly connected to the first container (e.g. glued and/or welded) so that the dosing and grinding device and the first container are firmly connected to each other. However, it is also conceivable that the dosing and grinding device and the first container are detachably connected to each other.

This enables the first container with coffee beans and the dosing and grinding device to be introduced into the first receiving area in a simplified manner and at least partially received by the first receiving area. At the same time, the dosing and grinding device can safely engage with the drive device, so that the intended amount of coffee beans can be introduced or dosed from the first container into the dosing and grinding device and then ground into coffee powder. However, it is also conceivable that the dosing and grinding device and the first container are not connected to each other and are introduced into and/or removed from the first receiving area separately from each other.

Preferably, the dosing and grinding device comprises a grinding device, the grinding device being designed for dosing and grinding.

The grinding device can comprise a grinder. By operating the grinder, the coffee beans can be dosed and ground at the same time. A separate dosing device, for example a screw conveyor (as described later), can be dispensed with.

Preferably, a defined amount of coffee can be controlled or regulated and/or dosed by means of a control or regulation of the grinding device or the grinder.

For example, by actuating the grinder, a defined amount of coffee beans can be ground over a certain period of time and thus a defined amount of coffee or ground coffee can be dosed at the same time. This enables the simultaneous grinding of coffee beans and dosing of a defined amount of ground coffee.

Preferably, a control or regulating parameter is a signal from a sensor device, wherein the sensor device comprises a scale and/or a timer.

The sensor device can comprise a timer. The timer can emit a signal by means of which the duration of grinding and dosing can be controlled or regulated. The grinder can be operated for a certain period of time via the timer, so that a defined amount or a predetermined amount of coffee beans can be ground and a certain amount of coffee powder can be dosed. Alternatively or at the same time, the sensor device can comprise a scale, for example a platform scale. The scale can, for example, be arranged below the container into which the ground coffee powder is filled, so that the weight inside the container can be determined. After reaching a certain or desired weight, the scale can send a signal to the grinding device so that grinding and dosing can be ended. The desired dosage is then achieved. It is also conceivable that the scale is arranged to the side or above the container into which the ground coffee powder is filled and, for example, designed as a hanging scale. The scale can for example be designed as a hanging scale and arranged above the container with coffee beans. The container with coffee beans can hang or be arranged on the scales so that the desired dosage can be determined by means of the weight or the weight loss of the container.

Preferably, the grinder can be inserted, preferably over its full length, into an outlet of the first container and is arranged rotatably therein, so that the grinder and the outlet extend around a common longitudinal axis.

The grinder can in particular be arranged at least partially in the outlet of the first container with coffee beans. In this way, the coffee beans inside the first container can be fed gravimetrically to the outlet and ground. With this arrangement, a screw conveyor, as described later, can be dispensed with. The coffee beans are transported to the grinder by gravity alone. The grinder is designed to then grind the coffee beans, i.e. after they have been gravimetrically transported to the grinder, to form a coffee powder.

Preferably, the grinder is rotatably arranged in the outlet of the first container. The grinder can be connected or connectable to the container. For example, the grinder can be glued into the outlet of the container or to the inner walls of the outlet. However it is also conceivable that the grinder is connected in one piece to the container.

When inserted into the outlet, the grinder and the outlet can extend around the same longitudinal axis. The grinder can be actuated and/or driven by the actuating and/or drive device. By actuating and/or driving the coffee beans from the first container can be ground by the grinder so that the ground coffee powder can leave the container or the grinder through the outlet of the first container.

The grinding mechanism can have a first end and an opposite second end along a grinding mechanism longitudinal axis. The first end can protrude from the outlet of the first container and thus be arranged outside of the first container. The second end can be disposed within the first container. The first end of the grinder can be designed as a drive end of the grinder.

At the drive end of the grinder, a coupling device can be arranged along the longitudinal axis of the grinder, or a coupling device can be arranged at the first drive end. The coupling device can comprise a transmission, for example a gear transmission with a gear or pinion, by means of which the grinding device or the grinder can be driven.

Preferably the grinder is a cone grinder. The cone grinder can be used to dose and grind at a low speed, preferably between 30 and 240 rpm (revolutions per minute).

The grinder can have a grinder core with an essentially conically shaped longitudinal section in the direction of the longitudinal axis of the grinder. The grinder core can extend between the first end and the second end of the grinder in the direction of the longitudinal axis of the grinder. Corresponding to the conically shaped longitudinal section of the grinder core, the circumference of the grinder core, seen transversely to the grinder longitudinal axis, decreases from the first end in the direction of the second end.

The grinder may have an inner ring adjoining or near to the second end. The inner ring can at least partially extend around the grinder core from the second end in the direction of the first end. The inner ring can surround the longitudinal axis of the grinding mechanism and preferably have a essentially conical longitudinal section along the longitudinal axis of the grinding mechanism, wherein the cross-sectional area of the inner ring can taper towards the second end.

The inner ring of the grinder seated on the grinder core or on the shaft can be moved by means of an adjusting element along the longitudinal axis of the grinder, in the direction of the first end and/or in the direction of the second end of the grinder. The adjusting element is arranged adjoining to or near to the first end and concentrically surrounds the longitudinal axis of the grinder. By means of the adjustment element, the inner ring can be displaceable in the direction of the first end and/or in the direction of the second end of the grinder. This enables a grinding degree to be set in a simple manner.

The grinder can have an outer ring. This outer ring can have an essentially cylindrical cross section and an inner circumference which is larger than the outer circumference of the inner ring and which is smaller than the inner circumference of the outlet of the first container. The outer ring can be arranged on the inner wall of the outlet of the first container or can be arranged adjoining or near to the inner wall of the outlet. The outer ring can be arranged on the inner wall of the outlet by means of a holding element, for example a hold-down device. The outer ring can be held in a fixed or stationary position in the outlet by the hold-down device.

The outer ring can be arranged around the inner ring so that the inner ring can rotate within the outer ring due to the drive of the grinding mechanism. By adjusting the degree of grinding by means of the adjusting element, the position of the inner ring relative to the outer ring (viewed in the direction of the longitudinal axis of the grinder) can be adjusted, so that a gap between the inner ring and the outer ring can be adjusted. Within the space, the coffee beans can be ground to coffee powder at the interfaces of the inner ring and the outer ring. The coffee beans, which are gravimetrically conveyed to the outlet and the grinder, thus get into the space between the inner ring and the outer ring and can be ground into coffee powder due to the rotation of the inner ring within the outer ring. The inner ring and the outer ring are arranged adjoining or near to the inner walls of the outlet or to the outlet opening of the first container. The coffee powder ground between the inner ring and the outer ring can thus exit the first container through the outlet.

The motor can be accommodated or arranged as part of the actuation and/or drive device in the dosage receiving area of the device. The motor can comprise a gearwheel or a pinion so that the gearwheel or the pinion of the motor can come into contact with the gearwheel or pinion of the coupling device of the grinder and the grinder or the grinder can be driven.

The motor can preferably be controlled or regulated by means of a signal from the sensor device.

The motor can communicate with the sensor device, for example the scales described above, so that the motor can be switched off after the desired dosage amount has been reached. The device can thus be operated in an automated manner.

Preferably, the device for dosing and grinding coffee beans and/or preparing coffee is designed such that the housing has only one receiving area, the receiving area being designed to receive the first container for coffee beans. In other words, the device does not include a second receiving area for receiving the second container for a fluid. The device can therefore also be designed without the second container for a fluid, so that the tempering device for tempering the fluid can be dispensed with.

Preferably, the dosing and grinding device comprises a grinding device and a dosing device. The grinding device can comprise a grinder and the dosing device can comprise a screw conveyor. Furthermore, the dosing and grinding device preferably comprises a screw conveyor, a grinder and a screw conveyor housing, the screw conveyor, preferably in its full length, being introduced into the screw conveyor housing and/or rotatably arranged or mounted therein, the grinder preferably being in its full length, is inserted into the screw conveyor housing and is rotatably arranged therein, so that the screw conveyor, the grinder and the screw conveyor housing extend around a common longitudinal axis of the screw conveyor housing.

The dosing and grinding device, thus, comprises a screw conveyor housing in which the grinder and the screw conveyor are arranged. In other words, the dosing and grinding device comprises a housing or a dosing and grinding device housing, which is referred to below as a screw conveyor housing. The screw conveyor is designed to transport the coffee beans to the grinder. The grinder is designed to then grind the coffee beans, i.e. after they have been transported to the grinder by means of the conveyor screw, to form a coffee powder.

The screw conveyor can be designed as a shaft around one or more helically wound flights in the form of flat metal sheets and/or rubber flaps or wings, which essentially extend in the form of a screw thread transversely away from the longitudinal axis of the conveyor screw. The screw conveyor is preferably designed as a rigid screw conveyor. However, it is also conceivable that the screw conveyor is designed as a flexible, in particular bendable screw. The screw thread can either be firmly connected to the shaft, for example welded, or it can be manufactured or manufactured in one part with the shaft. The screw conveyor preferably comprises a continuous, continuous screw thread which extends between the opposite ends of the screw conveyor along the longitudinal axis of the screw conveyor. This enables in particular the transport of coffee beans by means of the screw conveyor along its longitudinal axis. The screw conveyor, in particular the screw thread, can be turned from a solid material, for example from a piece of round steel, or as a cast part or Injection molded part are made. The screw conveyor and/or the screw conveyor housings are essentially cylindrical.

The configuration of the dosing and grinding device enables the coffee beans to be guided from the first container into the dosing and grinding device and transported by means of the screw conveyor in the screw conveyor housing along the longitudinal axis of the screw conveyor to the grinder. With each turn of the screw conveyor, a certain amount of coffee beans can be conveyed, so that the number of (partial) rotations can determine the dosage of the coffee beans and thus the coffee powder ground by the grinder.

This enables precise and simplified dosing of the coffee powder, which can take place both automatically, for example controlled by a regulating or control device, or manually.

The dosing and grinding device can comprise a screw conveyor with a screw conveyor and a screw conveyor housing, wherein a grinding device for coffee beans can be arranged in the screw conveyor housing adjoining or near to the screw conveyor. The screw conveyor can be connected or connectable to the grinder in such a way that the shaft can simultaneously drive the screw conveyor and the grinder to rotate. The longitudinal axis of the grinder and the longitudinal axis of the screw conveyor preferably extend in one plane or in a straight line.

The dosing and grinding device and/or the grinder can be designed as disposable or single-use-articles. Thus, the grinding surfaces or grinding knives of the grinder do not have to be ground or replaced after a certain period of use. Rather, the entire grinder with the dosing and grinding device or the packaging can be exchanged so that a high grinding quality or grinding quality can be firmly guaranteed.

The grinder can for example be made of ceramic or comprise ceramic.

Preferably, the screw conveyor housing has an inlet with an inlet opening and an outlet with an outlet opening. The inlet and the outlet are preferably arranged in the screw conveyor housing on opposite sides, viewed transversely to the longitudinal axis of the screw conveyor.

Through the inlet opening in the inlet, coffee beans can be fed from the first container into the interior of the screw conveyor housing in order to be taken up by one or more helically wound flights of the screw conveyor. The device for preparing coffee can comprise a shaking device with which the first container or its contents can be set in a shaking motion. This enables the coffee beans to be guided almost completely out of the first container through the inlet opening into the interior of the screw conveyor housing, especially if the coffee beans do not slide by themselves and are to be guided into the interior of the screw conveyor housing, for example by gravity. The shaking device can preferably be arranged in or corresponding to the first receiving area. The device for preparing coffee can comprise at least one sensor element or camera element. The shaking device or the shaking function can be controllable or regulatable via a regulating or control device and/or a sensor element or a camera element. A similar shaking device or shaking function can also be provided for the container or the filter container into which the ground coffee powder is fed from the outlet of the screw conveyor housing so that the coffee powder is evenly distributed in the container. A sensor element or a camera element can be used to determine the time or duration in which the desired amount of fluid has entered the container or filter container. Based on this period of time, the degree of grinding can be adjusted automatically or manually. Since the degree of grinding is decisive for the quality and the aroma of the coffee, its automatic adjustment enables the preparation of a particularly good or particularly aromatic coffee.

Preferably, the inlet is arranged adjoining or near to the screw conveyor and the outlet is arranged adjoining or near to the grinder.

As a result of the rotation of the screw conveyor, after entering the interior of the screw conveyor housing, the coffee beans are conveyed by the screw conveyor essentially along the longitudinal axis of the conveyor screw to the grinder. After the coffee beans have been ground by the grinder, the coffee powder can exit the screw conveyor housing through the outlet and preferably be brought into contact with the fluid for preparing coffee.

Preferably, the grinder is a cone grinder. The cone grinder can grind at a low speed, preferably between 30 and 240 rpm (revolutions per minute).

Preferably, the screw conveyor has a screw pitch diameter, i.e. an outer diameter transverse to the longitudinal direction of the screw conveyor, which is in a range of approximately 20 to 40 mm. The screw flank diameter is particularly preferably approximately 25 mm. This dimensioning of the screw flank diameter favors the conveyance or dosage of the coffee beans.

Preferably, the conveyor screw has a length which lies in a range between approximately 50 and 120 mm. The length of the screw conveyor is particularly preferably between approximately 50 mm and 90 mm, further preferably approximately 65 mm. The screw conveyor preferably has a diameter which lies in a range between approximately 10 and 40 mm. The diameter is particularly preferably between approximately 20 and 30 mm, more preferably the diameter of the screw conveyor is approximately 22 mm. This dimensioning of the length of the screw conveyor favors the conveying of the coffee beans. If the length of the screw conveyor is reduced, the coffee beans can form bridges in the one or more helically wound flights, so that the inlet opening is blocked and no further coffee beans can be introduced through the inlet opening. The bridging can occur particularly when the coffee beans are to be guided through the inlet opening into the screw conveyor housing by means of gravity.

Preferably, the screw conveyor housing has a length which lies in a range between approximately 100 mm and 140 mm. The length of the screw conveyor housing is preferably between approximately 105 mm and 120 mm, more preferably approximately 110 mm. Preferably, the screw conveyor housing has a diameter which is at least as large or is slightly larger than the diameter of the screw conveyor and/or the grinder. Preferably, the screw conveyor housing has a diameter which is in a range between approximately 25 and 50 mm. The diameter of the screw conveyor housing is particularly preferably between approximately 27.5 and 35 mm, more preferably the diameter of the screw conveyor housing is approximately 30 mm.

Dimensioning the length and the screw flank diameter of the screw conveyor in the value ranges described above enables a delivery rate of coffee beans in the range of around 1 to 5 g per revolution of the screw conveyor (e.g. around 2 g per revolution). The number of revolutions (or the angle of rotation around the longitudinal axis) allows the desired amount of coffee beans to be ground and thus the desired amount of ground coffee powder to be guided through the outlet of the screw conveyor housing and thus out of the screw conveyor housing. This enables precise dosing of the coffee beans or the ground coffee powder for the preparation of coffee.

Preferably, the inlet opening is essentially oval-shaped and extends in the direction of the longitudinal axis. However, other shapes of the inlet opening are also conceivable. The inlet opening comprises a length in the range from approximately 20 mm to 60 mm (e.g. from approximately 47 mm) in the direction of the longitudinal axis of the conveyor screw and/or a length in the range from approximately 10 mm to 40 mm (e.g. from approximately 29 mm) transversely to the conveyor screw longitudinal axis, in particular seen perpendicular to the longitudinal axis of the screw conveyor. Preferably, the outlet opening is essentially rectangular and extends in the direction of the longitudinal axis. However, other forms of the outlet opening are also conceivable. The outlet opening comprises a length in the range from approximately 20 mm to 50 mm (e.g. from approximately 30 mm) in the direction of the longitudinal axis of the conveyor screw and/or a length in the range from approximately 5 mm to 20 mm (e.g. from approximately 10 mm) across the longitudinal axis, in particular seen perpendicular to the longitudinal axis of the screw conveyor. These dimensions of the inlet opening and outlet opening enable a particularly favorable introduction of coffee beans into the screw conveyor housing and a particularly favorable execution of ground coffee powder from the screw conveyor housing.

Preferably, the screw conveyor housing extends between a first end and an opposite second end along the longitudinal axis of the screw conveyor housing, wherein the grinder is arranged adjoining or near to the first end and extends along the longitudinal axis of the grinder, wherein the screw conveyor is arranged adjoining or near to the second end and extends along the longitudinal axis of the screw conveyor, the outlet being arranged adjoining or near to the first end and the inlet being arranged adjoining or near to the second end. The longitudinal axis of the screw conveyor, the longitudinal axis of the grinder and the longitudinal axis of the screw conveyor housing preferably extend in one plane or in a straight line.

The inlet and the outlet are preferably arranged at a distance from each other in the longitudinal direction of the screw conveyor housing. By arranging the inlet adjoining or near to the second end and arranging the outlet adjoining or near to the first end of the screw conveyor housing, the coffee beans can be received by one or more helical flights after entering the interior of the screw conveyor housing through the inlet opening in the inlet are conveyed by the rotation of the screw conveyor to the second end of the screw conveyor housing, picked up by the grinder and ground to coffee powder so that the ground coffee powder can exit again through the outlet opening. Thus, a predetermined or predeterminable amount of coffee beans can be conveyed per revolution, so that a dosage can be set (or controlled or regulated) based on the number of revolutions (or the angle of rotation around the longitudinal axis).

The first end of the screw conveyor housing is preferably designed to be open and the second end of the screw conveyor housing is preferably designed to be closed. Thus, the screw conveyor can be inserted completely into the screw conveyor housing through the first end, preferably until it reaches the second end. Subsequently, the grinder can be completely inserted through the first end into the screw conveyor housing, preferably up to one end of the screw conveyor. But it is also conceivable that the screw conveyor and the grinder are designed in one piece, so that the screw conveyor and the grinder can be completely inserted as a unit into the screw conveyor housing, preferably until they reach the second end. An insertion element or a removal element, which extends away from the second end, can be provided at the second end. The insertion element or removal element can be designed as a flap which comprises a surface that is approximately the size of a thumb. In particular, the insertion element or removal element can have a length of approximately 3 to 4 cm and/or a width of approximately 2 to 3 cm. On opposite sides, the insertion element or removal element can comprise a haptic corrugated structure. The corrugated structure is preferably made of a soft, rubberized material. However, it can also be made of the same material as the insertion element or removal element.

By means of the insertion element, the dosing and grinding device can be held and/or introduced into the dosing and grinding device receptacle in a targeted manner. Furthermore, the dosing and grinding device can also be easily removed again by means of the insertion element, in particular if the first container is empty and has to be replaced.

Preferably, the inlet comprises a flange with a peripheral wall which at least partially surrounds the inlet opening and extends (preferably essentially radially) away from the screw conveyor housing, the flange for connecting the dosing and grinding device to the first container and/or for introducing the dosing and grinding device is configured in the dosing and grinding device receiving area.

The peripheral wall of the inlet in the screw conveyor housing is designed to be able to engage with the first container, in particular with an outlet in the first container. This enables the coffee beans from the first container to be introduced into the screw conveyor housing in a particularly reliable manner. The peripheral wall can be manufactured in one piece with the screw conveyor housing, or it can be manufactured as a cast part or injection-molded part that can be connected to the screw conveyor housing.

The peripheral wall can extend away from the edge of the inlet opening in the screw conveyor housing essentially at an angle different from 0° or 180°, in particular transversely. The peripheral wall, like the inlet opening, can thus be essentially oval-shaped and extend in the same direction as the longitudinal axis of the conveyor screw. However, other shapes are also conceivable for the peripheral wall. In particular, the peripheral wall has essentially the same shape as the inlet opening. The peripheral wall can have a circumference in the range of approximately 100 mm to 130 mm (e.g., approximately 122 mm). The peripheral wall can extend along a first peripheral wall central longitudinal axis, which can have a length in the range of approximately 30 mm to 60 mm (e.g., approximately 47 mm). Furthermore, the peripheral wall can extend along a second peripheral wall central longitudinal axis, which is oriented perpendicular to the first peripheral wall central longitudinal axis, and/or can have a length in the range from approximately 20 mm to 40 mm (e.g. from approximately 29 mm). Other lengths are also possible. The length of the first peripheral wall central longitudinal axis is preferably greater than the length of the second peripheral wall central longitudinal axis. The previously described lengths of the first and second peripheral wall central longitudinal axes are particularly favorable for introducing the coffee beans into the screw conveyor housing and/or for connecting the dosing and grinding device to the first container.

Preferably, the peripheral wall comprises a first contact surface and an opposing second contact surface, the first and second contact surfaces being aligned parallel to each other.

The first and second contact surfaces can be arranged on opposite sides of the second peripheral wall central longitudinal axis. These contact surfaces allow a particularly simple introduction of the dosing and grinding device into the dosing and grinding device receiving area. In particular, during insertion into the dosing and grinding device receiving area, the contact surfaces can slide along lateral guide elements in the first receiving area and, after being received in the dosing and grinding device receiving area, can rest against the lateral guide elements. The first contact surface and the second contact surface can have an essentially parabolic cross-sectional area. Due to the design of the two contact surfaces and the lateral guide elements, as well as their interaction when the first container is inserted into the first receiving area, the first container can be received in a correct position by the first receiving area so that the ground coffee powder can be guided out of the outlet of the dosing and grinding device in the correct dosage.

Preferably, a coupling device extends from a drive end of the screw conveyor in the direction of the longitudinal axis of the screw conveyor and a coupling device extends from the drive end of the grinder in the direction of the longitudinal axis of the grinder. The coupling device of the screw conveyor is designed to interact in a coupling manner with an actuating and/or drive device of the grinder, in particular to intervene. The coupling device of the grinder is designed to interact in a coupling manner with the actuating and/or drive device for the dosing and grinding device, in particular to intervene.

The coupling device of the screw conveyor is designed to interact in a coupling manner with the actuating and/or drive device of the grinder, in particular to intervene, or to be connected. In the interconnected state, the coupling device of the screw conveyor engages with the actuating and/or drive device of the grinder in such a way that the longitudinal axes of the grinder and the screw conveyor run in one plane or in a straight line, and when inserted into the screw conveyor housing with the longitudinal axis of the screw conveyor housing extend in a plane or in a straight line. Opposite to that of the actuation and/or drive device of the grinder, the grinder has a coupling device. The coupling device of the grinder is designed to interact with the actuating and/or drive device of the device for dosing and grinding coffee beans and/or for preparing coffee in a coupling manner, in particular to intervene or to be connected. This is advantageous because the grinder and the screw conveyor can be driven simultaneously via the same shaft by actuating or driving the actuating and/or drive device of the device. However, it is also conceivable that the grinder does not have an actuating and/or drive device and that the screw conveyor does not have a coupling device, but that instead the grinder and the screw conveyor are integrally connected to each other and can be driven in a coupling manner together via the coupling device of the grinder, as previously described.

The coupling device of the screw conveyor can be designed as an essentially cylindrical cavity and/or as a receptacle which extends essentially in the direction of the longitudinal axis of the screw conveyor. Correspondingly, the coupling device of the grinder can be configured as an essentially cylindrical cavity and/or as a receptacle which extends essentially in the direction of the longitudinal axis of the grinder. After the dosing and grinding device has been introduced and received in the dosing and grinding device receiving area, a coupling element in the dosing and grinding device receiving area can simultaneously be received in the (preferably essentially cylindrical) cavity of the grinder. The inner wall of the (cylindrical) cavity of the screw conveyor preferably has an inner profile which can be brought into engagement with an outer profile of the outer wall of the coupling element of the grinder. The inner wall of the (cylindrical) cavity of the grinder preferably has an inner profile which can be brought into engagement with an outer profile of the outer wall of the coupling element of the device.

For example, the outer profile of the coupling element of the device can have at least one material elevation which can engage or interact with at least one material depression in the inner profile of the cylindrical cavity of the grinder. Correspondingly, the outer profile of the coupling element of the grinder can have at least one material elevation which can engage or interact with at least one material depression in the inner profile of the cylindrical cavity of the conveyor screw.

The coupling element of the device can be designed as a drive shaft, so that the introduction of the coupling element into the cylindrical cavity of the grinder enables the dosing and grinding device to be driven and thus a rotation of the grinder and the screw conveyor when the grinder and the screw conveyor are by means of the coupling element the grinder and the coupling device or cavity of the screw conveyor are connected to each other. Preferably, the translation of the speed is adjustable or variable. This enables a change in the speed of the coffee beans conveyed through the screw conveyor housing and thus a change in the dosage of the coffee beans and subsequently ground coffee powder.

Preferably, the grinder has a grinder core with an essentially conically shaped longitudinal section in the direction of the longitudinal axis of the grinder. The grinder or the grinder core can be designed as a shaft. The grinder core has a first end and an opposite second end, the coupling device being arranged at the first end and the coupling element, which can be connected to the coupling device of the screw conveyor, being arranged at the second end. Corresponding to the conically shaped longitudinal section of the grinder core, the circumference of the grinder core, seen transversely to the longitudinal axis of the grinder, decreases from the first end in the direction of the second end. At no point on the grinder core, viewed in the direction of the longitudinal axis of the grinder, does the grinder core have a circumference which exceeds the circumference of the screw conveyor, seen in the direction of the longitudinal axis of the screw conveyor. This enables the grinder and the screw conveyor can be introduced together into the screw conveyor housing, so that the grinder and the screw conveyor can be driven together to rotate approximately the longitudinal axis of the screw conveyor housing by the actuating and drive device of the device for grinding and dosing coffee beans.

Preferably, the grinder has an inner ring adjoining or near to the second end of the grinder core. The inner ring can extend at least partially around the grinder core from the second end in the direction of the first end. The inner ring can surround the longitudinal axis of the grinding mechanism and preferably has an essentially conical longitudinal section along the longitudinal axis, the cross-sectional area of the inner ring tapering towards the second end of the grinding mechanism core. The inner ring of the grinder seated on the grinder core and/or the shaft can be moved by means of an adjusting element, for example by means of an adjusting screw, along the longitudinal axis of the grinder, in the direction of the first and the second end of the grinder core. The adjusting element is preferably arranged adjoining or near to the first end of the grinder core and surrounds the longitudinal axis of the grinder concentrically. The position of the inner ring can be adjusted in the direction of the longitudinal axis of the grinder by means of the adjusting element. The inner ring can thus easily be displaced in the direction of the first end and/or in the direction of the second end of the grinder core. This enables a degree of grinding to be set in a simple manner. By means of the adjustment element, the degree of grinding can be adjusted to the type of coffee beans in the first container and/or to the type of preparation (e.g. Chemex, Cold Brew, Karlsbader). The degree of grinding is decisive for the quality and aroma of the coffee. As finer the degree of grinding as longer the fluid or water runs through the coffee powder. This makes the coffee or the extraction stronger. As coarser the grind, as faster the fluid or water runs through the coffee powder. If the grind is too coarse, the coffee can taste watery and/or sour. If the grind is too fine, the coffee can become too strong, taste earthy and incompatible. The correct grinding degree must therefore be set so that the coffee tastes as good as possible and as many good aromas as possible can develop.

It is conceivable that the first container with coffee beans can be scanned by means of a scanning element, for example a smartphone, so that the degree of grinding can be set or regulated or controlled automatically or automatically (by means of a regulating or control device). For example, the degree of grinding can be adjustable depending on the volume of the fluid in the second container, or the degree of grinding can be adjustable depending on the desired type of preparation of the coffee (e.g. Chemex, Cold Brew, Karlsbader). The dosing and grinding device can have the grinder suitable for the coffee beans contained in the first container and only needs to be inserted or clicked into the dosing and grinding device receptacle in the device. The degree of grinding can also be set manually using the adjusting element. It is also conceivable, however, that the grinding degree is already preset, preferably adapted to the type or type of coffee beans, so that it does not have to be set automatically and/or manually.

Preferably, the grinder has a spring element which is arranged adjoining or near to the inner ring and/or adjoining or near to the second end of the grinder core. The spring element can for example be arranged on the rear part of the shaft or the grinder. The rear part of the shaft means the second end of the grinder, on which the actuation and drive device of the grinder is arranged. However, it is also conceivable that a recess within the grinder core extends from the second end of the grinder core at least partially in the direction of the first end. This recess can be arranged at a distance from the longitudinal axis or the central longitudinal axis of the grinder core and extend essentially concentrically around the longitudinal axis of the grinder. Thus, the distance between the recess and the outer wall of the grinder core, which is surrounded by the inner ring, transversely to the longitudinal axis or central longitudinal axis of the grinder core, can be less than the distance to the longitudinal axis or central longitudinal axis of the grinder core. With this arrangement, the spring element can also be arranged in the recess and thus ensure that the inner ring is in the selected position for the setting of the desired grinding degree.

Preferably, the grinder has an outer ring. This outer ring can have a essentially cylindrical cross-section with an inner circumference that is larger than the outer circumference of the inner ring. The outer ring is preferably arranged on the inner wall of the screw conveyor housing, more preferably the outer ring is arranged on the inner wall of the screw conveyor housing by means of a holding element, for example a hold-down device. The hold-down device can extend between the first open end of the screw conveyor housing, adjoining or near to the adjusting element, up to the outer ring along the inner wall of the screw conveyor housing.

Preferably, the outer ring of the grinder has an outer diameter which is in a range between approximately 20 and 30 mm, preferably in a range from approximately 25 to 27 mm, more preferably the outer diameter is approximately 25.7 mm. The outer ring of the grinder preferably has an inner diameter which is in a range between approximately 10 and 20 mm, preferably in a range from approximately 17 to 19 mm, more preferably the inner diameter is approximately 18 mm. The outer ring preferably has a length along which the central longitudinal axis of the outer ring extends, which is in a range between approximately 5 and 15 mm, preferably in a range between approximately 8 and 12 mm, more preferably the length is approximately 11 mm.

Preferably the, grinder core has a diameter which is in a range between approximately 10 and 25 mm, particularly preferably in a range between approximately 13.5 mm and 19.5 mm. The grinder core preferably has a length which is in a range between approximately 5 and 15 mm, particularly preferably between approximately 10 and 12 mm. More preferably, the length of the grinder core is approximately 11.1 mm.

Thus, the outer ring can be arranged around the inner ring so that the inner ring can rotate within the outer ring due to the drive of the grinder.

By adjusting the degree of grinding by means of the adjusting element, the position of the inner ring relative to the outer ring (viewed in the direction of the longitudinal axis of the conveyor screw or in the direction of the longitudinal axis of the screw conveyor housing) can be adjusted so that there is a gap between the inner ring and the outer ring and/or the interface between the inner ring and the outer ring can be adjustable. Due to the essentially conically shaped inner ring, the coffee beans can be ground to coffee powder at the interfaces between the rotating inner ring and the stationary outer ring. The coffee beans conveyed by the screw conveyor in the direction of the grinder thus reach the space between the inner ring and the outer ring and can be ground into coffee powder due to the rotation of the inner ring within the outer ring.

Preferably, the inner ring and the outer ring are arranged adjoining or near to the outlet or to the outlet opening of the screw conveyor housing. Thus, the coffee powder ground between the inner ring and the outer ring of the grinder can leave the screw conveyor housing through the outlet.

Preferably, the grinder and the screw conveyor can be driven in the interconnected state with a drive force of approximately 0.5 Nm to 2 Nm, preferably approximately 1 Nm, in order to convey the coffee beans by means of the screw conveyor towards to the grinder and subsequently grind them by means of the grinder according to the set grinding degree.

Preferably, the screw conveyor housing comprises an outer wall with a plurality of ribs, the ribs preferably extending essentially in the axial direction at least partially between the first end and the second end, and/or wherein the ribs extend away from the outer wall essentially in the radial direction.

The ribs are preferably formed as longitudinal ribs between the first and second end and/or surround the outer wall in the peripheral direction at regular or symmetrical intervals. The ribs can extend away from the outer wall so that each of the ribs has an outer edge that runs in a straight line that runs essentially parallel to the longitudinal axis of the screw conveyor housing and/or has a essentially constant distance from the outer wall of the screw conveyor housing. However, the ribs can, also have an e.g. conically shaped area, which is preferably near to the first end of the screw conveyor housing. In this conically shaped area, the outer edge of the ribs tapers towards the first end of the screw conveyor housing.

Preferably, two more of the ribs limit the outlet opening on or on opposite sides in the peripheral direction of the outer wall. In other words, two of the ribs are arranged adjoining or near to the outlet opening and extend away from the edge of the outlet opening. Two further ribs are preferably provided, which limiting the outlet opening on opposite sides in the axial direction of the outer wall. These further ribs run between the two ribs limiting the outlet opening on opposite sides in the peripheral direction and are arranged adjoining or near to the outlet opening, wherein they extend away from the edge thereof. The outlet opening can thus be surrounded by ribs on all sides.

The ribs on the outlet, in particular on the outlet opening, advantageously prevent the exiting coffee powder from coming into contact with the housing of the device for dosing and grinding coffee beans and/or for preparing coffee. Because the coffee powder does not touch the housing of the device, the housing does not have to be cleaned after each use and can be reused immediately. In addition, it is avoided that the coffee powder on the housing is contaminated and/or cannot be used to prepare coffee. The ribs can, however, also serve as a stand for the dosing and grinding device, in particular if the dosing and grinding device is not inserted in the dosing and grinding device receiving area of the first receiving area. This enables a simple connection of the first container to the dosing and grinding device and a subsequent simple filling of the first container with coffee beans.

Preferably, the first receiving area has a rear wall, more preferably the first receiving area has two side walls which are spaced apart from each other and which are oriented at an angle different from 0° or 180°, in particular essentially transversely to the rear wall. Preferably, the first receiving area has an upper and a lower limitation which are oriented at an angle different from 0° or 180°, in particular essentially transversely to the side walls. Further preferably, the first receiving area also has an open front side opposite the rear wall, so that the first receiving area is formed between the side walls and/or the upper and lower limitation. The first receiving area preferably has a container receiving area for receiving the first container, the container receiving area preferably being arranged above the dosing and grinding device receiving area.

The container receiving area can thus adjoin the upper limitation and/or the dosing and grinding device receiving area can adjoin the lower limitation. Through the open front side, the first container together with the dosing and grinding device can thus be introduced into the first receiving area by an essentially perpendicular movement to the rear wall, so that the first container is received by the container receiving area and the dosing and grinding device is received from the dosing device and grinder receiving area. Preferably, the first container is connected to the dosing and grinding device in such a way that the first container, when inserted into the first receiving area, is arranged above the dosing and grinding device relative to the lower limitation and/or is spaced further from the lower limitation than the dosing and grinding device. This enables the coffee beans to be guided from the first container into the dosing and grinding device, for example by gravity.

Preferably, a first guide element and a second guide element are arranged between the container receiving area and the dosing and grinding device receiving area, the guide elements extending essentially from the open front to the rear wall and/or wherein the guide elements extend away from the side walls.

The guide elements can run essentially continuously from the front to the rear wall. They allow a particularly simple introduction of the first container and the dosing and grinding device in the interconnected state into the first receiving area, so that the first container is arranged and/or received above the guide elements and the dosing and grinding device is arranged and received below the guide elements. For the correct insertion of the first container and the dosing and grinding device, the peripheral wall can be inserted between the guide elements, so that the first and second support surfaces slide essentially along the guide elements. In other words, the first support surface slides along the first guide element and the second support surface slides along the second guide element until the dosing and grinding device is completely received by the dosing and grinding device receptacle. In the state inserted into the dosing and grinding device receiving area of the first receiving area, the lateral contact surfaces of the peripheral wall of the dosing and grinding device then rest on the two guide elements. This enables the first container and/or the dosing and grinding device to be received in a particularly simple manner and to be arranged in a stable manner in the first receiving area.

Preferably, the guide elements are aligned essentially in a plane parallel to the upper limitation and/or to the lower limitation, the guide elements preferably being inclined towards the front out of the plane towards the container receiving area.

As a result, the guide elements each include an insertion bevel adjoining or near to the open front side, which enables an aid for the correct insertion of the dosing and grinding device. In particular, during the introduction, two of the ribs which are arranged on the outer wall of the screw conveyor housing can slide essentially along the underside of the guide elements, while the two lateral contact surfaces slide between the guide elements as previously described. In the state inserted into the dosing and grinding device receiving area of the first receiving area, the lateral contact surfaces of the peripheral wall of the dosing and grinding device and two of the ribs then rest on the two guide elements. In particular, the abutment surfaces may abut the edges of the guide elements which extend away from the side walls, and the two ribs can rest against the underside of the two guide elements facing towards the lower limitation.

When the screw conveyor or the screw conveyor housing is inserted into the dosing and grinder receiving area, the screw conveyor can click into place, e.g. as soon as the end position has been reached. This means that the user knows that the screw conveyor has been installed correctly or that the (cylindrical) cavity has been correctly connected to the coupling element or the drive shaft. The lead-in bevels can help to bring the first container into the correct position and/or also simplify the click-in of the screw conveyor.

Preferably, the lower limitation has a receptacle for the screw conveyor housing extending from the open front to the rear wall.

The receptacle for the screw conveyor housing can extend between the two side walls around a longitudinal axis of the receptacle which is oriented essentially parallel to the two side walls. On the longitudinal axis of the receptacle, in particular adjoining or near to the rear wall, a receptacle outlet opening may be arranged which is formed with essentially the same shape and dimension as the outlet opening of the screw conveyor housing. The receptacle has a cross section transverse to the receptacle longitudinal axis which is essentially concave. In other words, the receptacle is embedded as a essentially concave section in the lower limitation. The lower limitation can thus have a surface that has a first horizontal surface portion adjoining or near to a first of the side walls and a second horizontal surface portion adjoining or near to the second of the side walls, wherein the receptacle as a essentially concave surface portion between the first and second surface section is arranged.

The receptacle for the crew conveyor housing enables the dosing and grinding device to be held particularly securely and firmly in the dosing and grinding device receptacle. After the dosing and grinding device has been inserted and received, the screw conveyor housing lies firmly in the receptacle for the screw conveyor housing, with two of the ribs resting firmly on the first and second horizontal surface sections. At the same time, the outlet opening in the screw conveyor housing is arranged above or adjoining or near to the receiving outlet opening. The ground coffee powder can be conveyed from the screw conveyor housing through the outlet opening in the screw conveyor housing and the receptacle outlet opening in the receptacle in the dosing and grinding device receptacle and, for example, fed to a preparation device without the coffee powder coming into contact with the housing.

Preferably, the drive shaft is formed in or on the rear wall, the drive shaft and the receptacle extending in a plane transverse to the lower limitation.

The coupling element or the drive shaft is preferably arranged in or on the rear wall. The distance between the lower limitation and the coupling element or the drive shaft seen in a plane transverse to the longitudinal direction of the receptacle can correspond to the distance between the first cylindrical cavity and the peripheral wall of the screw conveyor in a plane seen transversely to the longitudinal direction of the screw conveyor. By inserting the dosing and grinding device into the receptacle for the screw conveyor housing as previously described, the coupling element or the drive shaft can automatically engage in the (cylindrical)

The plurality of ribs preferably extends essentially parallel to the upper and/or lower limitation. The plurality of ribs preferably extends essentially from the open front to the rear wall. However, it is also conceivable that the ribs are arranged transversely the upper or lower limitation and/or that the ribs do not extend continuously from the front to the rear wall.

The plurality of ribs is preferably arranged in pairs on the two side walls. In other words, two ribs each extend in a plane transversely to the side walls and/or parallel to the upper or lower limitation. In this way, numerous pairs of ribs can be arranged on the side walls in the container receiving area, preferably between the guide elements and the upper limitation. Preferably, the ribs of a pair of ribs are each spaced between approximately 40 and 50 mm from each other, more preferably the ribs of a pair of ribs are each approximately 50 mm apart. It is also conceivable that not all ribs of the rib pairs have the same distance from each other, but can have a different distance from each other, preferably between approximately 40 and 50 mm.

The ribs enable optimal alignment of the first container received in the first receiving area, so that the coffee beans can be guided and/or dosed from an outlet in the first container through the inlet opening of the screw conveyor housing. At the same time, this prevents coffee beans from remaining in the first container and from being unable to be used for the preparation of coffee. Thus, the ribs allow a plurality of differently shaped first containers to be easily and securely received and thereby brought into a certain desired shape, so that the powder can shift in the direction of the outlet. The first container is thereby held in a position, in particular in an upright position, in which it does not collapse.

Preferably, a container for receiving and dosing and grinding coffee beans is provided, the container comprising a housing having an interior space for receiving coffee beans and an outlet in fluid communication with the interior space, which can be connected to an inlet of a dosing and grinding device wherein the dosing and grinding device has an outlet, so that by actuating the dosing and grinding device, the coffee beans are dosed and ground to a coffee powder, so that the coffee powder is dispensed through the outlet. The container is preferably designed to be introduced into a device for dosing and grinding coffee beans and/or for preparing coffee, as previously described, and to be at least partially received by this. The dosing device is or can be connected to the container, and the container and/or the dosing device and grinding device are exchangeable and designed as disposable articles.

Preferably, the container for receiving and dosing and grinding coffee beans can be supplied pre-filled with coffee beans. The container can be delivered filled with coffee beans from the factory, i.e. the container can be filled with coffee beans at the factory so that the container can already be delivered to the consumer filled with coffee beans.

The container can have all of the features and advantages of the first container as previously described. The container can be designed to be introduced as the first container into the first receiving area of the above-described device for dosing and grinding coffee beans and/or for preparing coffee and to be at least partially received therein. Thus, all the previously described features of the device, which were described in connection with the first container and/or the dosing and grinding device, also apply to the container described below (hereinafter referred to as the first container) for receiving, dosing and grinding coffee beans.

The first container may include a dosing and grinding device to which the first container is connectable and thus may be connected so that the first container and the dosing and grinding device are used to dose and grind the correct amount of coffee beans into coffee powder. Thus, it is possible that a correctly dosed amount of coffee powder is provided by means of the dosing and grinding device. However, it is also conceivable that the first container and the dosing and grinding device are separate elements. Because the first container has an outlet with an outlet opening, coffee beans that are received in the first container can exit or be dispensed from the first container. Because the outlet can be connected to the inlet of a dosing and grinding device, the coffee beans emerging from the first container can be introduced into the dosing and grinding device through the inlet. By means of the screw conveyor, the coffee beans can then be transported along the longitudinal axis of the screw conveyor to the grinder, so that the coffee powder ground by the grinder can exit the second outlet in the dosing and grinding device and can be used in the specified or predeterminable dosage for the preparation of coffee powder.

Thus, with the described first container a correct dosing of coffee beans and a grinding to a coffee powder by means of the dosing and grinding device can be performed. The dosing and grinding device can be driven by an actuating and/or drive device. However, it is also conceivable that the dosing and grinding device is driven manually. Fluid from the second container can be mixed with the coffee powder, which is obtained by means of the coffee beans exiting the first container, and introduced in a correct mixing ratio into a container, in particular into a filter container. This allows coffee to be prepared correctly and in a simplified manner.

Furthermore, the first container may comprise a dosing and grinding device to which the first container is connectable and thus may be connected so that the first container and the dosing and grinding device may be used to dose the correct amount of coffee beans and, consequently, to dispense freshly ground coffee powder in a correctly dosed manner. For this purpose, the first container connected to the dosing and grinding device can also be inserted and received in the first receiving area, in particular in the container receiving area and the dosing and grinding device receiving area of the device for preparing coffee described above. However, it is also conceivable that the first container and the dosing and grinding device are two separate elements, each of which is inserted into the first receiving area, in particular into the container receiving area and into the dosing and grinding device receiving area, and received separately from each other.

Thus, a correct dosing of coffee beans from the first container and subsequent grinding into coffee powder can be performed by means of the dosing and grinding device. The dosing and grinding device can be driven by an actuating and/or driving device which is arranged, for example, in a dosing and grinding device receiving area in the device described above. However, it is also conceivable that the dosing and grinding device is driven manually. The coffee powder dosed and ground by the dosing and grinding device can be introduced into a container, in particular into a filter container, in a correct mixing ratio, together with a fluid that is provided for example from the second container. This enables the coffee to be prepared correctly and in a simplified manner.

Preferably, the dosing and grinding device comprises a grinding device, the grinding device being designed for dosing and grinding.

The grinding device can comprise a grinder. By operating the grinder, the coffee beans can be dosed and ground at the same time. A separate dosing device, for example a screw conveyor (as described later), can thus be dispensed with.

Preferably, a defined amount of coffee can be controlled or regulated and/or dosed by means of a control or regulation of the grinding device or the grinder.

For example, by actuating the grinder, a defined amount of coffee beans can be ground over a certain period of time and thus a defined amount of coffee or ground coffee can be dosed at the same time. This enables the simultaneous grinding of coffee beans and dosing of a defined amount of ground coffee.

Preferably, the first container comprises a sensor device and/or the first container is connectable, in particular signal-connectable, to a sensor device.

Preferably, a control or regulating parameter is a signal from the sensor device, wherein, preferably, the sensor device comprises a scale and/or a timer.

The sensor device can comprise a timer. The timer can emit a signal by which the duration of grinding and dosing can be controlled or regulated. Thus, the grinder can be operated for a certain period of time via the timer, so that a defined amount or a predetermined amount of coffee beans can be ground and a certain amount of coffee powder can be dosed. Alternatively or at the same time, the sensor device can comprise a scale, for example a platform scale. The scale can, for example, be arranged below the container into which the ground coffee powder is filled, so that the weight can be determined inside the container. After reaching a certain or desired weight, the scales can send a signal to the grinding device so that grinding and dosing can be ended. The desired dosage is then achieved. It is also conceivable that the scale is arranged to the side or above the container into which the ground coffee powder is filled and is designed, for example, as a hanging scale. The scale can for example be designed as a hanging scale and arranged above the container with coffee beans. The container with coffee beans can hang or be arranged on the scales so that the desired dosage amount can be determined by means of the weight or the weight loss of the container.

Preferably, the grinder can be introduced into the outlet of the first container, preferably over its full length and is rotatably arranged therein, so that the grinder and the outlet extend around a common longitudinal axis.

In particular, the grinder may be arranged at least partially in the outlet of the first container of coffee beans. In this way, the coffee beans inside the first container can be gravimetrically guided to the outlet and ground. With this arrangement, a screw conveyor, as described later, can be dispensed with. The coffee beans are transported to the grinder by gravity alone. The grinder is designed to then grind the coffee beans, i.e. after they have been gravimetrically transported to the grinder, to form a coffee powder.

Preferably, the grinder is rotatably arranged in the outlet of the first container. The grinder can be connected or connectable to the container. For example, the grinder can be glued into the outlet of the container or to the inner walls of the outlet. However, it is also conceivable that the grinder is integrally connected to the container.

When inserted into the outlet, the grinder and the outlet can extend around the same longitudinal axis. The grinder can be actuated and/or driven by the actuating and/or drive device. By actuating and/or driving the coffee beans from the first container can be ground by the grinder so that the ground coffee powder can leave the container or the grinder through the outlet of the first container.

The grinder can have a first end and an opposite second end along a longitudinal axis of the grinder. The first end can protrude from the outlet of the first container and thus be disposed outside of the first container. The second end can be arranged inside the first container. The first end of the grinder can be designed as a drive end of the grinder.

At the drive end of the grinder, a coupling device can be arranged along the longitudinal axis of the grinder, or a coupling device can be arranged at the first drive end. The coupling device can comprise a transmission, for example a gear transmission with a gear or pinion, by means of which the grinding device or the grinder can be driven.

The grinder can have a grinding mechanism core with an essentially conically shaped longitudinal section in the direction of the longitudinal axis of the grinder. The grinder core can extend between the first end and the second end of the grinder in the direction of the longitudinal axis of the grinder. Corresponding to the conically shaped longitudinal section of the grinder core, the circumference of the grinder core, seen transversely to the grinder longitudinal axis, decreases from the first end in the direction of the second end.

The grinder may have an inner ring adjoining or near to the second end. The inner ring can extend at least partially around the grinder core from the second end in the direction of the first end. The inner ring can surround the longitudinal axis of the grinding mechanism and preferably have an essentially conical longitudinal section along the longitudinal axis of the grinding mechanism, wherein the cross-sectional area of the inner ring can taper towards the second end.

The inner ring of the grinder seated on the grinder core or on the shaft can be moved by means of an adjusting element along the longitudinal axis of the grinder, in the direction of the first end and/or in the direction of the second end of the grinder. The adjusting element is arranged adjoining or near to the first end and concentrically surrounds the longitudinal axis of the grinder. By means of the adjusting element, the inner ring can be displaceable in the direction of the first end and/or in the direction of the second end of the grinder. This enables a grinding degree to be set in a simple manner.

The grinder can have an outer ring. This outer ring can have an essentially cylindrical cross section and an inner circumference which is larger than the outer circumference of the inner ring and which is smaller than the inner circumference of the outlet of the first container. The outer ring can be arranged on the inner wall of the outlet of the first container or can be arranged adjoining or near to the inner wall of the outlet. The outer ring can be arranged on the inner wall of the outlet by means of a holding element, for example a hold-down device. The outer ring can be held in a fixed or stationary position in the outlet by the hold-down device.

The outer ring can be arranged around the inner ring so that the inner ring can rotate within the outer ring due to the drive of the grinder. By adjusting the degree of grinding by means of the adjusting element, the position of the inner ring can be adjusted relative to the outer ring (viewed in the direction of the longitudinal axis of the grinder), so that an intermediate space between the inner ring and the outer ring can be adjusted. Within the space, the coffee beans can be ground to coffee powder at the interfaces of the inner ring and the outer ring. The coffee beans, which are gravimetrically conveyed to the outlet and the grinder, thus get into the space between the inner ring and the outer ring and can be ground into coffee powder due to the rotation of the inner ring within the outer ring. The inner ring and the outer ring are arranged adjoining or near to the inner walls of the outlet or to the outlet opening of the first container. Thus, the coffee powder ground between the inner ring and the outer ring can exit the first container through the outlet.

Preferably, the actuating and/or drive device for the dosing and grinding device comprises a motor, the motor being designed to drive the grinder.

The motor can be accommodated or arranged as part of the actuation and/or drive device in the dosage receiving area of the device. The motor can comprise a gearwheel or a pinion so that the gearwheel or the pinion of the motor can come into contact with the gearwheel or pinion or sprocket of the coupling device of the grinder and the grinding device or the grinder can be driven.

Preferably, the motor can be controlled or regulated by means of a signal from the sensor device.

The motor can communicate with the sensor device, for example the scales as previously described, so that the motor can be switched off after the desired dosage amount has been reached. In that way the device can be operated in an automated manner.

Preferably, the first container and/or the second container comprise at least partially a flexible material and/or at least partially a dimensionally stable material.

Preferably, the housing of the first container and/or the housing of the second container comprises an aluminum composite film or is formed from an aluminum composite film.

Preferably, the first container, for example its outer wall, comprises at least one valve. Preferably, the at least one valve is configured to release carbon dioxide from the first container. Preferably, the at least one valve is designed so that no oxygen penetrates into the container.

Preferably, the dosing and grinding device comprises a screw conveyor, a grinder and a screw conveyor housing, wherein the screw conveyor, preferably in its full length, is insertable and rotatable into the screw conveyor housing, the grinder being inserted into the screw conveyor housing, preferably in its full length and is rotatably arranged therein, so that the screw conveyor, the grinder and the screw conveyor housing extend around a common longitudinal axis of the screw conveyor housing, and wherein the inlet of the dosing and grinding device is arranged in or on the screw conveyor housing.

Thus, the design of the dosing and grinding device enables the coffee beans to be guided from the first container into the dosing and grinding device and transported by means of the screw conveyor in the screw conveyor housing along the longitudinal axis of the screw conveyor towards the grinder and then ground to coffee powder by the grinder. With each turn of the screw conveyor, a certain amount of coffee beans can be conveyed, so that the dosage of the coffee beans or the ground coffee powder can be determined by the number of revolutions. This enables precise and simplified dosing of the coffee beans or the ground coffee powder, which can be done automatically, for example controlled by a regulating or control device, or manually by an operator.

The first container can be connected to a dosing and grinding device comprising a screw conveyor, a grinder and a screw conveyor housing. The screw conveyor, the grinder and the screw conveyor housing can thereby have all the features that were previously described in the context of the device for dosing and grinding coffee beans and/or for preparing coffee, so that the dosing and grinding device can be received in the first receiving area or can be received in the dosing and grinder receiving area of the device as previously described.

It is conceivable that the dosing and grinding device comprises a plate, which is preferably designed as a stand plate and is arranged on the screw conveyor housing. This stand plate is used to better position the first container and/or to protect it from falling over, particularly if the first container for holding coffee beans is positioned outside the device for dosing and grinding coffee beans and/or for preparing coffee. The plate can be firmly connected to the screw conveyor housing or the plate can be connectable to the screw conveyor housing. Thus, after the coffee beans have been picked up, the plate can be removed from the screw conveyor housing, so that the first container and/or the dosing and grinding device can be accommodated in the first receptacle of the device for dosing and grinding coffee beans and/or for preparing coffee. It is further conceivable that the screw conveyor housing comprises a casing, the casing having at least one flat surface which serves as a stand plate, so that the first container can be better positioned and protected from falling over. Preferably, the outlet of the first container is firmly connected to the inlet in the screw conveyor housing, in particular screwed and/or glued.

The first container can be connected to the screw conveyor housing so that coffee beans can be introduced from the first container into the screw conveyor housing, so that after the conveyance has been carried out by the screw conveyor to the grinder and after grinding to coffee powder, the coffee powder is dispensed from this in the correct dosage can. The outlet of the first container can be firmly connected (e.g. glued) to the inlet of the screw conveyor housing. For this purpose, for example, the outlet of the first container can have a peripheral wall which is similar to the peripheral wall of the flange which is arranged on the screw conveyor housing. In particular, the peripheral wall of the container outlet can have a cross-sectional profile which corresponds to the cross-sectional profile of the peripheral wall of the flange, although the circumference of the peripheral wall of the container outlet is slightly larger or slightly smaller than the circumference of the peripheral wall of the flange. In this way, the peripheral walls can be brought into an overlap and/or firmly connected to each other (e.g. glued and/or welded).

However, it is also conceivable that the outlet of the first container is screwed to the inlet in the screw conveyor housing. Thus, the peripheral wall of the flange on the screw conveyor housing can comprise a first drive profile and the peripheral wall of the container outlet can comprise a second drive profile. Preferably, the first container and the dosing and grinding device can be connected to each other in a form-fitting and rotationally fixed manner via the two drive profiles. For example, the outer contour of the peripheral wall of the flange on the screw conveyor housing can have a drive profile and the inner contour of the peripheral wall of the container outlet can have a corresponding drive profile, so that the peripheral walls can be connected to each other in a non-rotatable manner in particular. Any structure that enables a connection between the first container and the dosing and grinding device can serve as the drive profile. The drive profile can accordingly be polygonal, star-shaped, slot-shaped, etc.!

Preferably, the screw conveyor housing is integrated into the first container. By integrating the screw conveyor housing into the first container, the first container and the screw conveyor housing can be connected integrally with each other, so that the first container and the dosing and grinding device are in particular firmly and non-detachably connected to each other. It is conceivable that in particular the peripheral wall of the container outlet and the peripheral wall of the flange on the screw conveyor housing are formed integrally with each other.

Preferably, the first container has at least partially a tapering section, the periphery of the first container in the tapering section preferably decreases essentially conically towards the outlet.

The first container can have a cross-section in a plane through the conveyor screw longitudinal axis of the conveyor screw conveyor housing when connected to the first container, the tapering section being laterally limited by a first side edge and a second side edge. In the “state connected to the first container” means that the dosing and grinding device or the screw conveyor housing are connected to the screw conveyor and the first container. The first side edge can run essentially transversely, preferably at an angle smaller than 90°, particularly preferably at an angle of approximately 45°, to the longitudinal axis of the conveyor screw conveyor housing (viewed in the connected state). The second side edge can run essentially transversely, preferably at an angle smaller than approximately 90°, particularly preferably at an angle of approximately 45°, to the longitudinal axis of the conveyor screw conveyor housing. It is also conceivable that both side edges run essentially transversely, preferably at an angle smaller than approximately 90°, particularly preferably at an angle of approximately 45°, to the longitudinal axis of the conveyor screw conveyor housing. Due to this arrangement of the side edges relative to the longitudinal axis of the screw conveyor housing (seen in the connected state) enables the coffee beans to be emptied particularly easily from the first container.

Preferably, the second side edge encloses an angle of approximately 45° with the first side edge. As a result of this configuration, the circumference of the first container in the tapering section is gradually reduced towards the outlet. This enables a particularly efficient emptying of the coffee beans received in the first container from the outlet and the subsequent introduction into the inlet of the conveyor screw conveyor housing.

Preferably, the first container has at least partially comprises a first essentially symmetrical section, wherein the circumference of the first container remains the same within the first essentially symmetrical section and wherein preferably a first essentially symmetrical portion is spaced further from the outlet than the tapering section.

The first container may have a cross-section in a plane through the conveyor screw longitudinal axis of the screw conveyor housing as viewed in the connected state with the first container, wherein the first essentially symmetrical section is laterally limited by a first side edge and a second side edge, which are aligned essentially parallel to each other and thus run essentially transversely, preferably at an angle of approximately 90°, to the conveyor screw longitudinal axis of the screw conveyor housing (as viewed in the connected state). The first side edge of the first essentially symmetrical section can run in one plane with the first side edge of the tapered section and/or the second side edge of the first essentially symmetrical section can be oriented transversely to the second side edge of the tapered section. However, it is also conceivable that the second side edge of the first essentially symmetrical section runs in the same plane as the second side edge of the tapering section, so that a further tapering section is formed instead of the symmetrical section.

Preferably, the distance between the first and second side edges of the symmetrical section is a maximum of approximately 140 mm and/or the length of the two side edges is a maximum of approximately 155 mm. It is also conceivable that the length of the first side edge is longer than the length of the second side edge. Thus, the length of the first side edge can be a maximum of approximately 155 mm and/or the length of the second side edge can be a maximum of approximately 125 mm. However, it is also conceivable that the previously described distances and lengths can differ from the specified values, so that the first container can have a smaller or a larger volume or can be smaller or larger.

Through this embodiment further enables particularly efficient emptying of the coffee beans accommodated in the first container out of the outlet of the first container and subsequent introduction into the inlet of the screw conveyor housing. At the same time, the symmetrical section enables alternative configurations of an inlet for receiving coffee beans in the first container.

Preferably, the first container has a second essentially symmetrical section adjoining or near to the outlet, wherein the periphery of the first container remains the same within the second essentially symmetrical section and essentially corresponding to the circumference of the outlet and/or an outlet opening in the outlet.

The first container can have a cross-section in a plane through the conveyor screw longitudinal axis of the conveyor screw conveyor housing when connected with the first container, the second essentially symmetrical section being laterally limited by a first side edge and a second side edge which are aligned essentially parallel to each other and thus run essentially transversely, preferably at an angle of approximately 90°, to the conveyor screw longitudinal axis of the conveyor screw conveyor housing (viewed in the connected state). The first side edge of the second essentially symmetrical section may be coplanar with the first side edge of the tapering section and with the first side edge of the first essentially symmetrical section, and/or the second side edge of the second essentially symmetrical section may be oriented transverse to the second side edge of the tapering section and parallel to the second side edge of the first.

Preferably, the distance between the first and second side edges of the second symmetrical section is in the range from approximately 20 mm to 60 mm (e.g. approximately 50 mm) and/or the length of the two side edges is in each case in the range from approximately 10 mm to 110 mm (e.g. approximately 15 mm or 90 mm each). However, it is also conceivable that the previously described distances and lengths can differ from the specified values, so that the first container can have a smaller or a larger volume or can be smaller or larger.

Preferably, the second essentially symmetrical section is connected to the outlet, so that further preferably the diameter of the outlet or the passage of the outlet opening corresponds to the distance between the first and second side edges of the second symmetrical section.

Through this embodiment further enables particularly efficient emptying of the coffee beans received in the first container from the outlet and subsequent introduction into the inlet of the screw conveyor housing.

However, it is also conceivable that the first container has a further, essentially symmetrical section instead of the tapering section. Here, the first side edges of the three sections can run in one plane and the second side edges can run in one plane, the two planes being oriented essentially parallel to each other.

Preferably, the first container has an inlet opening, the inlet opening preferably being arranged essentially opposite the outlet and/or an outlet opening in the outlet.

The inlet opening can preferably be arranged in the first essentially symmetrical section. Further preferably, the inlet opening can be arranged adjoining or near to a side edge which runs between the first and second side edges of the first essentially symmetrical section. The inlet opening is preferably arranged at a first free end of the first container which lies opposite a second free end of the first container, the outlet and the outlet opening being arranged at the second free end. The tapering section can be arranged between the inlet or the inlet opening and the outlet or the outlet opening.

Through the inlet opening coffee beans can be received in the first container. Due to arranging the inlet opening opposite to the outlet, the coffee beans can be guided in the direction of the outlet and the outlet opening and can be guided out of the first container into the dosing and grinding device. This enables correct dosage of the coffee beans or the ground coffee powder. Preferably, the inlet opening can be closed by means of a closure element, more preferably by means of a zipper.

However, it is also conceivable that the first container does not have an inlet opening and is integrally or firmly connected to the dosing and grinding device. The first container and the dosing and grinding device can be connected to each other in one piece as a unit and can be filled with coffee beans.

The inlet opening preferably extends adjoining to/or near to the first free end between the first and second side edges of the first essentially symmetrical section. Preferably, the inlet opening can be closed with a closure element. The first container is advantageously reusable and/or coffee beans can be refilled after they have been completely emptied, or the first container can be closed again after coffee beans have been transferred. However, it is also conceivable that the first container cannot be reused and does not have a closure element, as after the receiving of the coffee beans the inlet or the inlet opening is welded. It is also conceivable that the first container does not have an inlet or an inlet opening, but that the coffee beans are initially received into the first container through the outlet or the outlet opening, and the outlet is then connected to the dosing and grinding device. In particular, after the coffee beans have been taken up, the outlet can be connected to the inlet of the dosing and grinding device by means of a connecting element, for example an adhesive element in the form of an adhesive strip or a clip. In this case, one and the same opening is used to receive the coffee beans in the first container and to remove the coffee beans from the first container.

Thus, the first container with coffee beans can already be supplied connected to the dosing and grinding device and is designed as a disposable or single-use-article. It is also conceivable that the dosing and grinding device, which can be connected to the first container, is designed as a reusable article. In particular if the dosing and grinding device and the first container are formed integrally with each other or are glued or screwed to each other, the dosing and grinding device can be designed as a single-use or disposable article.

The closure element can be designed as a zipper that is easy to open and close. However, it is also conceivable that instead of the zipper or in addition to the zipper, a rail is arranged on the first free end of the first container. With this rail, the first container can be connected to an upper area of the first receiving area. It is also conceivable, that one or more magnetic holders, one or more Velcro fasteners, one or more buttons and/or one or more adhesive strips or other types of fastenings with which the first container can be connected to the upper area of the receiving area are also conceivable. It is further conceivable that the first container has a first screw element and the upper area of the first receiving area has a second screw element, such that the first container can be connected to the upper area of the receiving area by means of the screw elements.

A tab can be arranged adjoining to or near to the closure element. The tab may have an internal opening. The inner opening can be designed as a handle, so that the first container can be carried or held in a simplified manner from one location to another. The inner opening can, however, also serve to be hooked or suspended in a hook, for example, thereby providing additional stability, in particular when filling the first container. Preferably, the closure element, the zipper, is preferably designed to be inserted into a groove in a first receiving area of a device for dosing and grinding coffee beans and/or for preparing coffee.

The closure element or the zipper can be designed to be (at least partially) inserted into a groove. Preferably, the closure element or the zipper is designed to be inserted into a groove which is arranged in the first receiving area, in particular on the inner side the upper limitation which points towards the lower limitation. The groove may extend essentially in the same plane as the drive shaft in the dosing and grinding device receiving area and as the conveyor screw longitudinal axis of the conveyor screw conveyor housing when inserted into the device. Preferably, the groove extends at least partially in the upper limitation. Further preferably, the groove extends from an area adjoining or near to the open front side to an area adjoining or near to the rear wall. This enables the first container and/or the dosing and grinding device to be easily inserted into the first receiving area of the device for dosing and grinding coffee beans and/or for preparing coffee, the dosing and grinding device being received by the receiving device in the lower limitation and the cylindrical cavity of the dosing and grinding device can engage with the drive shaft in the drive device. At the same time, the closure element or the zipper can be inserted into the groove, which enables an additional hold for the first container in addition to the lateral ribs.

The first container can be made of different materials and can comprise, for example, paper, plastic or other flexible materials for holding coffee beans. Furthermore, the first container can be designed as a pouch or a bag. However, it is also conceivable that the first container is made of a non-flexible material and is therefore dimensionally stable, wherein it can possible comprise a metal such as aluminum or a plastic, for example. For example, the first container can also be designed as a cardboard box, for example a Tetra Pak. In particular, when the first container is made of a non-flexible material, the second receiving area can have two open sides, which are arranged on opposite sides of the longitudinal axis of the receiving area, instead of the closed side walls and the plurality of ribs which extend away from the side walls.

The first container can have a capacity of approximately 1.5 dm3. This capacity allows up to 500 g of coffee beans to be received, with 500 g of coffee beans corresponding to a volume of 1.1 dm3. Thus, the capacity of 1.5 dm3 allows convenient filling and/or transferring of coffee beans. However, it is also conceivable that the first container has a capacity that deviates from approximately 1.5 dm3, so that the first container can be made larger or smaller.

In the following the second receiving area and the second container are described in more details:

Preferably, the second receiving area has a rear wall, two side walls which are spaced apart from each other and which are aligned at an angle different from 0° or 180°, in particular essentially transversely to the rear wall, a lower limitation which is at an angle different from 0° or 180°, in particular is oriented transversely to the side walls, and an open top opposite the lower limitation, at least one of the side walls being an inclined side wall which is at an angle different from 90°, preferably at an angle between 10° and 50°, more preferably is oriented at an angle between 10° and 30°, particularly preferably at an angle of 20°, to the lower limitation.

The second receiving area can comprise an open top. In other words, the top surface can be designed to be completely open. This enables the second container to be introduced into the second receiving area by an essentially perpendicular movement to the lower limitation, so that the second container can be received by the second receiving area. However, it is also conceivable that the second receiving area has an upper limitation in which a through-hole or opening is arranged through which the second container can be introduced into the second receiving area by an essentially perpendicular movement to the lower limitation.

The second receiving area can have a front side opposite to the rear wall, which can preferably comprise a window element, for example a window element made of glass or plastic, or a flap or closure flap. This enables the filling level of the second container to be checked easily through the front. However, it is also conceivable that the front side is designed as a front wall which, like the rear wall, is closed and has no opening. The open front side of the first receiving area can also be closed by means of a flap or cap, preferably in a manner analogous to the closing flap of the second receiving area. Thus, after the first container has been inserted and picked up, the first receiving area can be protected from dust or dirt by closing the closure flap. Preferably, the at least one, second receiving area is designed to receive a lifting system for dosing fluid.

By means of the lifting system, a pressure on the second container for a fluid can be exerted, for example with the aid of a pump mechanism, so that the fluid can be dosed correctly. However, it is also conceivable that a pressure can be exerted on the second container with the aid of a rotating mechanism or some other mechanism, so that the fluid can be dosed correctly and in a particularly easy to implement manner. The second receiving area is designed in such a way, that it can also accommodate the lifting system in addition to the second container.

Preferably, the lifting system is connected or can be connected to the second container.

The lifting system can be firmly connected to the second container. In other words, the lifting system can be integrated into the second container and offered or delivered in such an integrated manner. However, it is also conceivable that the lifting system and the second container are two separate elements that can be combined or connected with each other so that the fluid can be dosed from the second container. For example, the lifting system can be combined or connectable to an opening of the second container, for example to the inlet or the outlet of the second container. Like the second container, the lifting system can be interchangeable and a disposable or single-use-article. Thus, the lifting system can be delivered ex works together with the second container, which is preferably already filled with fluid.

Preferably, the lifting system is connected or can be connected to the outlet of the second container. Thus, by operating the lifting system, the fluid can be pumped out of the second container and precisely dosed. Preferably, the lifting system is connected or connectable to a container or mug, for example a coffee pot or a coffee mug or one of the preparation devices described below, for example via a hose system. In this way, the correctly dosed fluid can be filled into the container or into the cup or into the preparation device.

Preferably, the container or cup or the preparation device is arranged in the direction of gravity below the lifting system that can be or is connected to the second container. Preferably, the lifting system is arranged or positioned in the direction of gravity between the container or cup or preparation device and the second container. Thus, the fluid can be guided in a simple manner from the second container in the direction of the lifting system by gravity and pumped out of the second container into the container or cup or to the preparation device, so that the fluid is particularly simple in the container or cup or can be guided into the preparation device.

The lifting system preferably has a piston and a rotary plate.

It is conceivable that the lifting system can the fluid by means of a piston pump. For example, the lifting system can comprise a rotary plate which is driven by means of a motor and can exert a pressure on a piston. The piston can be connectable or connected to the second container in such a way that the piston can be deflected or moved by the rotary plate. The rotary plate is preferably arranged above the piston in the direction of gravity, so that the rotary plate can be driven by the motor and set in a rotary motion. This rotary movement sets the piston in a translatory movement. Thus, the piston can be pressed downward in the direction of gravity in the direction of the second container so that the fluid can be pumped out of the second container and correctly dosed. Thus, by deflecting or moving the piston, the fluid can be pumped out of the second container in a simple manner and correctly dosed. One revolution of the motor can lead to several lifting movements. In other words, one revolution of the motor can lead to several combined rotary and translational movements of the rotary plate and the piston.

Preferably, the rotating plate is designed as an eccentric or as a control disk, which is attached on a shaft and whose center is outside the shaft axis. The piston is preferably arranged below the eccentric in the direction of gravity and outside, preferably above or below in the direction of gravity, its shaft axis. In this way, the rotational movement of the eccentric can advantageously be converted into the translational movement of the piston or into the piston stroke.

Preferably, the second receiving area has a lifting system receiving area.

The lifting system receiving area is designed to receive the motor and/or the rotary plate and/or the piston. It is conceivable that the motor and/or the rotary plate and/or the piston are firmly connected to the lifting system receiving area. These can be arranged, for example, on the rear wall of the second receiving area and/or on its side walls. The second container can then be introduced and inserted into the second receiving area in such a way that the rotary plate and/or the piston in the lifting system receiving area can interact or interact with the second container. Thus, the fluid can be pumped out of the second container and correctly dosed by means of the rotary plate and the piston. It is also conceivable that the motor and/or the rotary plate and/or the piston are firmly connected to the second container and can be exchanged together with the second container. Thus, the motor and/or the rotary plate and/or the piston can be inserted together with the second container into the lifting system receiving area, so that the fluid can be pumped and dosed out of the second container.

Preferably, the lifting system has a sensor.

The sensor can be firmly connected to the lifting system receiving area. The level of the fluid in the second container can be determined by means of the sensor when the second container is introduced or inserted into the second receiving area. The sensor can be connected to application software, for example a mobile app, as described later, so that new fluid can be ordered automatically on the Internet based on the fill level of the fluid.

Preferably, the second container is connected or connectable to a dosing device or the second container comprises a dosing device, wherein preferably the dosing device being a lifting system, the lifting system being designed to dose the fluid from the interior of the second container.

It is conceivable that the device comprises only one dosing and grinding device for dosing and grinding the coffee beans and no dosing device for dosing the fluid.

Preferably, the lifting system is made from a bioplastic or bioplastic or a bio-based plastic. Preferably, the lifting system comprises a bioplastic or bioplastic or a bio-based plastic. For example, the bioplastic can comprise stone paper and/or wood.

The inclined side wall of the second receiving area can preferably be connected or connectable to the rear wall and can be spaced from the lower limitation. Thus, the lower edge of the inclined side wall or the edge of the inclined side wall, which points to the lower limitation of the second receiving area, can be arranged adjoining or near to a flange which can surround a through-hole in the lower limitation. The inclined side wall is configured to receive and retain the second container of fluid in the inclined position. In other words, the second container can have one of its outer side walls abutting the inclined side wall such that the inclined side wall is configured as a support member and/or as a support member for the second container. The second container can be held in an inclined position by the inclined side wall, so that the outlet of the second container can open into the through-hole, which can be surrounded by a flange. The flange enables a simplified introduction of the outlet of the container into the through-hole and at the same time serves as a lateral support or as a lateral support element for the outlet. The inclined position of the second container is advantageous since it allows the fluid to flow out of the second container in a suitable manner, so that no residual volume or residual fluid or dead volume remains in the second container. At the same time, the first container with the dosing and grinding device can thus be arranged in a position which is arranged essentially transversely, preferably at an angle of 90° to the lower limitation and above the lower limitation and/or above the through-hole. In this way, the outlet of the screw conveyor housing and the outlet of the second container can advantageously open together in the through-hole. This enables the ground coffee powder and the fluid to be guided through the through-hole in the device for dosing and grinding coffee powder and/or for preparing coffee, so that the coffee powder and the fluid can be fed to a container, preferably a filter container.

Preferably, the outlet of the first container and the outlet of the second container open into the through-bore at a distance from each other. The distance between the two outlets when the first container is inserted and received in the first receiving area and when the second container is received in the second receiving area is preferably approximately 30 mm to 60 mm, more preferably approximately 45 mm.

However, it is also conceivable that the second receiving area has two side walls which are separate from each other and which are arranged transversely to the rear wall, preferably at an angle of approximately 90° to the rear wall, and are aligned parallel to each other. A third wall, which is designed as an inclined wall and has the features of the inclined side wall previously described, can be arranged between these two separate side walls.

Preferably, the second receiving area has a multiplicity of clamping elements which extend at least partially between the front side opposite the rear wall and the rear wall of the second receiving area. Preferably, the clamping elements are designed as clamps, wherein preferably the clamps are arranged in a plane parallel to the inclined side wall.

Particularly, preferably, at least two clamping elements, preferably three clamping elements, which run in a plane or clamping element plane parallel to the inclined side wall, are particularly preferably arranged adjoining or near to the inclined side wall of the second receiving region. However, it is also conceivable that the second receiving area has more than three clamping elements adjoining or near to the inclined side wall, which clamp elements run in the clamping element plane. Preferably, the clamping element plane is oriented at an angle different from 90°, preferably at an angle between 10° and 50°, more preferably at an angle between 10° and 30°, particularly preferably at an angle of 20° to the lower limitation. This is advantageous because the second container can thus be held between the inclined side wall and the clamping elements so that one of the side walls of the second container rests against the inclined side wall and an opposite side wall of the second container rests against the clamping elements. Thus, the fluid can be dosed out of the second container by means of the clamping elements. The clamping elements allow the fluid to be dosed in the interior of the second container or from the interior of the second container. The desired or correct dosage of the fluid in the interior of the second container can be clamped off by the clamping elements. This is advantageous because expensive peristaltic pump, flow sensors, etc. can be dispensed with.

Preferably, at least one of the clamping elements is replaced by the tempering device for controlling the temperature of the fluid to be dosed by means of the clamping elements.

By introducing the second container through the open top into the second receiving area, the second container is received in the second receiving area in such a way that the second container can be held and/or clamped laterally by the up to three clamping elements and the inclined side wall. The clamping elements clamp the fluid inside the second container. Because the clamping elements can be arranged displaceably on the rear wall of the second receiving area, the fluid can be dosed by moving the clamping elements. Thereby, the up to three clamping elements can assume at least one position, in particular a first position and a second position. In the first position, up to three clamping elements can laterally adjoin the second container and/or touch the second container in such a way that the clamping elements can exert pressure on one of the side walls of the second container and an opposite second side wall of the second container is against the inclined side wall pressed. In the second position, the up to three pairs of clamping elements cannot adjoin the container or touch the second container, so that the clamping elements cannot exert any pressure on one of the side walls of the second container. The different positions of the at least one clamping element are advantageous if the fluid in the interior of the second container is to be tempered or heated by means of the tempering device. Due to the heating caused by the heating or boiling, the fluid in the interior of the second container expands, so that the circumference of the outer wall and thus the distance between the side walls of the second container increases due to the expanding fluid.

Due to the first position and the second position of the at least one clamping element, the position of the clamping elements can be changed or displaced relative to the side walls or relative to the inclined side wall and/or relative to the lower limitation of the second receiving area. Thus, the position of the clamping elements can also be changeable relative to the side walls or relative to the inclined side wall and/or relative to the lower limitation of the second container when the second container is received in the second receiving area.

A first clamping element can be arranged such that the clamping element can have a first distance from the lower limitation. A second clamping element can be arranged in such a way that the clamping element can have a second distance from the lower limitation which is greater than the first distance from the lower limitation. The first clamping element can thus be arranged as a lower clamping element adjoining or near to the lower limitation in the second receiving area. The second clamping element can be arranged as an upper clamping element adjoining or near to the open upper side. Furthermore, a third clamping element can be arranged as a middle clamping element between the first clamping element and the second clamping element and can have a third distance from the lower limitation which is greater than the first distance and less than the second distance.

The first, lower clamping element can have a first distance to the lower limitation which is between 10 mm and 30 mm, preferably approximately 20 mm. The second, upper clamping element can have a second distance from the lower limitation which is between 160 mm and 240 mm, preferably approximately 180 mm. The distance between the first, lower clamping element and the second, upper clamping element can preferably be between 140 mm and 220 mm, preferably approximately 160 mm. The third, middle clamping element can be arranged between the first, lower clamping element and the second, upper clamping element so that it is displaceable in a range between 10 mm and 30 mm, preferably approximately 20 mm, and a range between 160 mm and 240 mm, preferably approximately 180 mm from the lower limitation.

The first, lower clamping element can exert pressure on one of the side walls of the second container in the state when the second container is received in the second receiving area, or the side walls of the second container are clamped or trapped between the lower clamping element and the inclined side wall so that the second container is closed and no fluid can escape from the second container, for example through an outlet opening in the second container, when the second container is received in the second receiving area. The first, lower clamping element can thus seal the second container in a sterile manner, so that no bacteria or germs can enter the second container, for example through the outlet opening in the second container. The second, upper clamping element can exert a pressure on one of the side walls of the second container or the side walls of the second container are clamped or clamped between the upper clamping element and the inclined side wall in such a way that the second container is closed and no fluid from the second container and/or from a fluid reservoir that can be connected to the second container, for example through an inlet opening in the second container, can emerge when the second container is received in the second receiving area. The second, upper clamping element can thus seal the second container in a sterile manner, so that no bacteria or germs can enter the second container, for example through an inlet opening in the second container. The inclined side wall with the heating element can serve as a counter surface to the clamps, preferably to the lower clamp, so that the second container can be arranged between the lower clamp and the heating element and/or the inclined side wall. It is also conceivable that the inclined side wall can serve for the other two clamps, i.e. the second, upper clamping element and/or the third, middle clamping element.

The first, lower clamping element and the second, upper clamping element close off the area to be sterilized or heated in the interior of the second container and thus store the fluid in a sterile manner. The third, middle clamping element is provided for dosing the fluid inside the second container between the first, lower clamping element and the second, upper clamping element.

Preferably, the first, lower clamping element and the second, upper clamping element are arranged in such a way that the two clamping elements delimit an area of the second container which delimits an amount of fluid of approximately 50 ml to 400 ml in the second container. This makes it possible to dose an amount of fluid up to approximately 400 ml. This area covers the amount of fluid that is required for a portion or for a cup of coffee and/or an espresso, depending on the preparation method required in each case. However, it would also be conceivable that the first, lower clamping element and the second, upper clamping element are arranged in such a way that the two clamping elements delimit a region of the second container, the one fluid amount of more than 400 ml, preferably of more than 400 ml and up to approximately 1000 ml or more than 400 ml and up to 750 ml, limit in the second container. In this way, an amount of fluid could also be provided or dosed which is suitable for preparing coffee for more than one serving or more than one cup of coffee, for example for several servings or a pot of coffee.

Preferably, the distance between the clamping elements can be changed relative to the lower limitation and/or relative to the open upper side.

Preferably, the third, middle clamping element is adjustable or height-adjustable in the clamping element plane. In other words, the third distance to the lower limitation can be changed. This enables the exact dosage of the desired amount of fluid for the preparation of coffee. It is also conceivable that the first, lower clamping element and the second, upper clamping element are adjustable or height-adjustable in the clamping element plane, so that the first and second distance to the lower limitation can be changed. This makes it possible to adapt the two clamping elements to the size or to the volume of the second container, so that two containers of different sizes can be received by the second receiving area and laterally held or limited by the clamping elements and the inclined side wall so that the fluid in the inside the second container can be correctly dosed.

Preferably, each of the clamping elements preferably comprises a first clamping element surface and a second clamping element surface, the clamping element surfaces being arranged on opposite sides of a clamping element longitudinal axis.

The first clamping element surface and the second clamping element surface can be arranged essentially parallel to each other and can each extend between a first end and a second end. The first clamping element surface can extend in a first plane and the second clamping element surface can extend in a second plane, wherein the first plane and the second plane are aligned parallel to each other and/or wherein the clamping element longitudinal axis is in a plane between the first plane and the second level is arranged. The width of the two clamping element surfaces, i.e. the width of the two clamping element surfaces at an angle different from 0° or 180°, in particular essentially transverse to the longitudinal axis of the clamping element, tapers from the first end to the second end. Furthermore, each of the clamping elements can comprise a connecting plate which is arranged at an angle different from 0° or 180°, in particular essentially transverse to the longitudinal axis of the clamping element. The first clamping element surface can be connectable to the connecting plate by means of the first end, and the second clamping element surface can be connectable to the connecting plate by means of the first end. The connecting plate is designed to connect the individual clamping elements to the second receiving area. In particular, the connecting plate can be connectable to the rear wall of the second receiving area so that the clamping elements extend essentially transversely to the rear wall at an angle different from 0° or 180°, in particular essentially transversely, so that the second ends of the clamping element surfaces are spaced from the rear wall. Preferably, the connecting plate of each of the individual clamping elements is connected to the rear wall adjoining or near to the inclined side wall of the second receiving area, so that the clamping elements extend along the inclined side wall, preferably in the clamping element plane, between the front side and the rear wall. This enables the second container to be held between the clamping elements and the inclined side wall after being received in the second receiving area and the fluid to be dosed inside the second container. Due to the fact, that the width of the two clamping element surfaces tapers towards the second end, the individual clamping elements can be brought from the first position to the second position in a particularly simple manner. However, it is also conceivable that the individual clamping elements are not connected to the rear wall by means of a connecting plate, but that the clamping elements are arranged or connectable in a displaceable manner on the rear wall and/or on one of the side walls of the second receiving area by means of a carriage or by means of a rail or guide rail element.

Preferably, the two clamping element surfaces are connected by means of a third clamping element surface, the third clamping element surface having an essentially conically shaped cross-section essentially transverse to the longitudinal axis of the clamping element.

The third clamping element surface can extend from a first side edge of the first clamping element surface to a first side edge of the second clamping element surface. The first side edges of the first and second clamping element surfaces can extend in the same plane, which extends at an angle different from 0° or 180°, preferably at an angle of 90°, in particular transversely to the longitudinal axis of the clamping element. The third clamping element surface can be arranged at an angle of 90° to the first clamping element surface and to the second clamping element surface and/or at an angle of 90° to the connecting plate and/or at an angle of 90° to the rear wall of the second receiving area when the connecting plate with the rear wall is connected. Preferably, the connecting plate comprises at least one through hole so that the clamping elements can be connected to the rear wall by means of a connecting element, for example a screw. However, it is also conceivable that the connecting plate is arranged adjoining or near to the first end of the clamping element surfaces, for example on second side edges opposite the first side edges of the clamping element surfaces, so that the clamping elements can be connected to the side walls.

Preferably, the third clamping element surface can have a essentially conically shaped or triangularly shaped cross-section at an angle different from 0° or 180°, in particular essentially transversely to the longitudinal axis of the clamping element. The third clamping element surface can have a clamping element edge which extends essentially in the direction of the clamping element longitudinal axis and due to the essentially conically shaped cross-section between the first side edge of the first clamping element surface and the first side edge of the second clamping element surface. Preferably, the clamping element edge extends in the same plane as the clamping element longitudinal axis. Due to the design of the clamping element edge of the individual clamping elements, the fluid in the second container can be particularly well dosed when the second container is received in the second receiving area and pressure is exerted on the side walls of the second container by means of the clamping elements and the inclined side wall.

It is also conceivable that the third clamping element surface comprises more than one clamping element edge, preferably two clamping element edges, which, like the previously described clamping element edge, extend essentially in the direction of the clamping element longitudinal axis and extend between the first side edge of the first clamping element surface and the first side edge of the second clamping element surface. The clamping element edges each extend in a plane which runs essentially transverse or at an angle different from 0° or 180°, preferably at an angle of 90°, to the plane of the clamping element longitudinal axis.

Each of the clamping elements can be designed to have an open configuration opposite the third clamping element surface and transverse to the clamping element longitudinal axis. In other words, each of the clamping elements comprises an inner cavity which is delimited by the three clamping element surfaces and has an open side. In the state of the clamping element connected to the second receiving area, for example when the clamping element is connected to the rear wall by means of the connecting plate, the open side of the inner cavity points to one of the two side walls of the second receiving area. Due to this configuration, the clamping elements have a reduced weight and are also suitable for clamping the second container and/or for dosing the fluid inside the second container. However, it is also conceivable that the clamping element has a fourth clamping element surface opposite the third clamping element surface, seen transversely to the clamping element longitudinal axis, which extends between a second side edge of the first clamping element surface and a second side edge of the second clamping element surface.

Preferably, at least one of the clamping element surfaces, preferably the third clamping element surface, is designed as a bearing surface, preferably as a rubberized support surface.

The bearing surfaces enable the second container to be closed particularly tightly. The bearing surfaces can be configured as rubberized bearing surfaces and comprise an elastomer or a thermoplastic or a thermoset or be made of such a material. The support surface can comprise a soft plastic or a solid plastic or be formed from a soft plastic or from a solid plastic.

In particular, when the first, lower clamping element and the second, upper clamping element have assumed the second position, the rubberized contact surface enables an improved seal so that no germs or bacteria can penetrate into the interior of the second container and the fluid is received in a sterile manner inside the second container and no fluid can escape from the second container. Furthermore, it is ensured that the interior of the device, in particular the interior of the second receiving area, for example the side walls, do not come into contact with the fluid. It is thus possible to dispense with cleaning the device, in particular cleaning the interior of the device.

Preferably, one or more (preferably each of the) clamping elements has at least one spring element.

The at least one spring element can be designed as a tension spring or as a rubber band, which is arranged adjoining or near to the first end of the first clamping element surface or adjoining or near to the first end of the second clamping element surface. However, it is also conceivable that a first spring element is arranged adjoining or near to the first end of the first clamping element surface and a second spring element is arranged adjoining or near to the first end of the second clamping element surface. The contact pressure of the clamping elements in the first state can be adjusted by the spring element. This enables a particularly tight closing or sealing of the second container, in particular by the first, lower clamping element and by the second, upper clamping element. The clamping element edge can be pressed particularly tight against one of the side walls of the second container, so that the fluid is received in a sterile manner in the second container. The spring support ensures a good and sterile seal.

A spring support enables a flat pressure or a uniform surface pressure, which enables a particularly sterile seal. The flat pressure or uniform surface pressure can exert a pressure on the contact surfaces of the clamping elements that can be greater than the hydrostatic pressure of the fluid in the interior of the second container or greater than the pressure resulting from the heating or boiling off the fluid. In this way, tightness or sealing of the second container is ensured at all times by means of the clamping elements and the spring support provided thereon.

Preferably, the tempering device is arranged in contact with the second container, wherein the tempering device preferably arranged in an area adjoining or near to the lower limitation of the second receiving area and/or wherein the tempering device is preferably arranged in an area adjoining or near to the inclined side wall and/or adjoining or near to one of the clamping elements, which is closest to the lower limitation, is arranged.

The tempering device can be arranged adjoining or near to the first, lower clamping element and/or adjoining or near to the lower edge of the second side wall, which points toward the lower limitation of the second receiving area and/or that on the flange that has the through hole in the lower limitation surrounds, can be applied. When the second container is received by the second receiving area, a lower area of the second container is arranged adjoining, preferably adjacent, to the tempering device. The tempering device can preferably comprise a tempering element, for example a heating plate, which is arranged adjoining or near to the lower limitation of the second receiving area and/or adjoining or near to the lower clamping element and/or adjoining or near to the lower edge of the inclined side wall. It is also conceivable that the tempering element is arranged between the first, lower clamping element and the third, middle clamping element, or that the temperature control element is arranged between the first, lower clamping element and the second, upper clamping element. In this case, the tempering element can extend between the first, lower clamping element and the third, middle clamping element or the second, upper clamping element. It is further conceivable that the device comprises more than one tempering element, preferably two tempering elements, which are each arranged adjoining or near to the inclined side wall.

This arrangement of the at least one tempering element allows the fluid to be mixed in the interior of the second container. The deepest or lowest point of the second container or the point of the second container which is closest to the lower limitation of the second receiving area when the second container is accommodated in the second receiving area is tempered or heated. In this way, a circulating movement of the fluid in the interior of the second container can be set in motion and the fluid is thoroughly mixed in the interior of the second container. This ensures that the fluid can be kept at the same temperature in the entire interior of the second container. This is advantageous since a mixing unit in the second container can be dispensed with.

Preferably, the tempering device comprises at least one sealing element, preferably two sealing elements. The at least one sealing element can be a sealing lip which is arranged adjoining or near to the lower limitation of the second receiving area and/or is arranged adjoining or near to one of the clamping elements of the first, lower clamping element pair and/or is arranged adjoining or near to one of the tempering elements. The sealing lip is designed to press the deepest, lowest point of the second container in the state received in the second receiving area, preferably the area adjoining or near to an outlet opening of the second container, against the tempering element, so that the tempering element is in contact, preferably in a surface-to-surface contact, is brought to the second container. However, it is also conceivable that instead of the separate sealing element, the first, lower clamping element can press the deepest, lowest point of the second container in the state received in the second receiving area, preferably the area adjoining to or near to an outlet opening of the second container, against the tempering element. This creates a particularly high thermal conductivity and the temperature of the fluid inside the second container is particularly effectively. When the second container is received in the second receiving area, the sealing element and the first, lower clamping element can be arranged on opposite sides of the second container.

The at least one tempering element can be designed as a heating element as previously described, for example as a heating plate, in order to set a circulating movement of the fluid in motion interior the second container and to heat the interior evenly. Preferably, the at least one tempering element is designed to heat the fluid to a temperature between 90° C. and 100° C., particularly preferably approximately 96° C. This enables the preparation of coffee.

However, it is also conceivable that the at least one tempering element is designed as a cooling element, for example as a cooling plate. Thus, the circulation movement in the interior can be stopped by the cooling element and the fluid can be cooled to a specified temperature. It is also conceivable that a first of the tempering elements is designed as a heating element, for example as a heating plate, and a second of the tempering elements is designed as a cooling element, for example as a cooling plate. Furthermore, one and the same tempering element can be designed both as a heating element and as a cooling element. As a result of the design as a cooling plate, the fluid in the interior of the second container can be cooled or chilled to a temperature which is advantageous for special types of coffee preparation methods, for example for cold brew preparation.

Preferably, the lower limitation of the second receiving area has a through hole.

The through hole is designed so that an outlet of the second container and/or an outlet of the first container can be passed through the through hole. In particular, when the second container is inserted through the open top into the second receiving area and received, an outlet at a lower end of the second container can be guided through the through hole, so that the outlet of the second container in the state received in the second receiving area through the through-hole is guided and protrudes below the second exception area. In the same way, the outlet from the screw conveyor housing can be guided through the through-hole so that the outlet of the screw conveyor housing is guided through the through-hole when it is received in the first receiving area and protrudes below the second receiving area. This enables the ground coffee powder to be guided through the outlet of the screw conveyor housing and the fluid through the outlet of the second container, the outlets being able to be guided by means of the through-hole in the direction of a container, for example a filter container. As previously described, the through-hole comprises a flange that laterally surrounds the through-hole and which extends away from the lower limitation in the direction of the upper limitation. This flange allows a simplified introduction of the outlets. At the same time, the outlets of the screw conveyor housing and of the second container can be designed to have a certain length, for example as extended outlets, so that the outlets can be guided through the through-hole in a simple manner without the fluid and the ground coffee powder already in the through-hole with each other come into contact or that the side walls of the through-hole are contaminated with coffee powder or with fluid. Thus, cleaning of the through-hole can thus be dispensed with. Preferably, the outlets of the screw conveyor housing and of the second container have a length which is greater than the length of the through hole and the flange, viewed transversely or at an angle of 90° to the lower limitation.

Preferably, the through-hole is arranged in the middle of the lower limitation of the device for dosing and grinding coffee beans and/or for preparing coffee, preferably at a point that is equidistant from a front and a rear of the device and/or the is equidistant from two opposite side walls of the device. A partition between the first receiving area and the second receiving area can then be dispensed with. The first container with the dosing and grinding device and the second container can then be arranged in such a way that the outlets can be guided through the through-hole.

This enables the outlets to be connected to the preparation device or to a container, for example a filter container, so that a correctly dosed amount of fluid can exit by means of the second container and by means of the preparation device or in the filter container with the preparation device as well or ground coffee powder fed to the filter container can be mixed and can be filled into a container, preferably into a coffee cup or into a coffee pot. The correctly dosed quantities of fluid and coffee powder can then be fed into a container or a filter container, and the ready-to-eat coffee can then be produced by shaking or jiggling the container or the filter container. In other words, the correctly dosed amount of fluid and coffee powder are mixed by shaking or jiggling the container or the filter container. Shaking or jiggling can be done manually by the user. However, it is also conceivable that the device has a shaking device and/or a mixing device, by means of which the correctly dosed fluid and coffee powder in the container or in the filter and/or funnel container are vibrated and/or mixed.

The coffee in the filter and/or funnel container can be mixed in order to obtain a homogeneous coffee. This can be done manually or by means of a rotating heating plate or by means of 3D acoustic waves, but possibly also by shaking. The shaking function can have several functions or is advantageous for several reasons: an advantageous sliding of the coffee powder or the coffee beans, an even distribution of the coffee powder in the coffee filter, whereby preferably a camera can be used to check the correctly distributed coffee, a recognition of the bloom effect, a mixing of the coffee powder in a dripping process or in cold brew, whereby preferably the mixing can take place via 3D acoustic waves, as well as recognition of how fast the water or fluid flows through the coffee powder, i.e. the flow rate of the fluid. The camera can thus recognize the bloom effect and/or the correct mixing of the coffee powder. If necessary, the grinding degree can be readjusted and adapted to the type of coffee or the type of coffee preparation (crema, dripping, etc.). This can be done automatically, so that the device is automatically optimized.

However, it is also conceivable that the mixing takes place by means of the preparation device. In case of the dripping method or the dripping type of preparation, the preparation device would be placed in a vessel which has a sieve at the bottom. The coffee drips from here into a container below, e.g. in a jug or in a vessel. In the case of the coldbrew, the preparation device would be in a container which is designed, for example, as a sieve, which in turn is inserted or can be inserted into another container with water. However, the mixture can only be optional. In other words, the device for dosing and grinding coffee beans and/or for preparing coffee can also only fill vessels or parts (in the correct dosage). In the case of the coldbrew method of preparation, the container can be filled with water and the filter coffee can be poured into the filter. In the case of the dripping type of preparation, the fluid or water and the coffee powder are mixed in the preparation device.

The following is a brief description of the features of the preparation device, which can be provided as an optional component in the device:

Preferably, the preparation device has an inner cavity which extends around a central longitudinal axis between an upper open end and a lower open end, the cavity being surrounded by an inner wall, the circumference of which preferably decreases from the upper open end to the lower open end. Preferably, the inner cavity comprises an inner wall which extends along the central longitudinal axis and divides the inner cavity into a first cavity region and a second cavity region. Preferably, a first closing flap for closing the first cavity area and a second closure flap for closing the second cavity area are arranged at the upper open end of the preparation device. Preferably, the preparation device comprises, adjoining or near to the upper open end, a connection for connecting or coupling the preparation device to the device and/or the preparation device comprises, adjoining or near to the lower open end, a connection for connecting or coupling the preparation device to a container, e.g. a jug or a vessel.

Preferably, the preparation device has a filter and/or funnel container into which the coffee powder and the fluid can be introduced and/or mixed. Furthermore, the preparation device can comprise a container, for example a coffee cup or a coffee pot, which is arranged relative to the filter and/or funnel container in such a way that the coffee is introduced or filled in from the filter and/or funnel container due to gravity can be. The coffee cup or the coffee pot is preferably arranged below the filter and/or funnel container. In the case of the dripping process, the preparation device can be placed in a vessel which has a sieve or a filter at the bottom at the bottom. The coffee drips from here into a container below, e.g. in a jug or vessel. In the case of the coldbrew method or type of preparation, the preparation device or stirrer and flaps of the preparation device would be in a container which is designed as a sieve, which is located in a further container in which fluid or water can be located.

Preferably, a drip tray is provided on the housing of the device, which drip tray extends from a side wall of the housing, preferably away from a rear wall of the housing. Preferably, the drip tray is arranged below the preparation device. Preferably, the distance of the first receiving area can be changed relative to the drip tray and/or the distance of the second receiving area can be changed relative to the drip tray. In particular, the housing of the device can thus be retractable or pushed together. This enables the device to be supplied, for example, with a pushed together or collapsible housing, so that packaging material for transport can be saved. Furthermore, changing the distance between the first receiving area and/or the second receiving area relative to the drip tray enables the distance to be adapted to the size of the container, in particular the coffee container into which the coffee is to be filled. Different sized containers or coffee containers can be arranged above or on the drip tray and filled with coffee.

Preferably, the device is designed to determine the presence and/or the type of preparation device.

Preferably, the dosing and grinding device comprises a closure or flap element, wherein a closure or flap element being designed to be opened automatically or manually, wherein preferably, the closure or flap element being designed to accommodate the dosing and grinding device and/or to seal the first container airtight.

Preferably, a container is provided for receiving and dosing fluid (in particular liquid) for preparing coffee, in particular filter coffee, wherein the container having a housing with an interior space for receiving fluid, an inlet in fluid connection with the interior space and an outlet in fluid connection includes with the interior space. Furthermore, the inlet can be connected to an outlet of a fluid reservoir and a dosing of the fluid for preparing coffee can be delivered through the outlet of the container. The container is replaceable and designed as a disposable article.

Preferably, the container for receiving and dosing fluid (in particular liquid) can be supplied pre-filled with fluid. The container can be supplied filled with the fluid from the factory, i.e. the container can be filled with fluid at the factory, so that the container can already be supplied to the consumer filled with fluid for the preparation of coffee, in particular filter coffee.

The fluid reservoir can be replaceable, i.e. the fluid reservoir can be designed as a disposable or single-use-article, just as the first container for coffee beans, the dosing and grinding device for dosing and grinding the coffee beans, the second container for a fluid and the preparation device can be designed as replaceable components. However, it is also conceivable that the respective components as previously described as replaceable are designed as reusable or reusable components. The fluid reservoir can be connected to the second container in such a way that the device for dosing and grinding coffee beans and/or for preparing coffee, in particular the second receiving area, does not come into contact with the fluid. The device, in particular the second receiving area, is thus not contaminated with fluid, so that cleaning of the device is not necessary after each individual preparation of coffee.

Preferably, the container is designed to be introduced into and received by a device for dosing and grinding coffee beans and/or for preparing coffee as a second container.

The container can be designed to be introduced as a second container into the second receiving area of the previously described device for dosing and grinding coffee beans and/or for preparing coffee and to be at least partially received therein. Thus, all the previously described features of the device that were described in connection with the second container therefore also apply to the second container described below for receiving and dosing fluid. In particular, the second container described below can be inserted and received in the second receiving area of the device described above, so that precise dosing of the fluid for preparing coffee is made possible by means of the clamping elements.

Preferably, the inlet of the second container comprises an inlet opening, which is preferably arranged essentially opposite the outlet of the second container viewed in the direction of a container longitudinal axis and/or essentially opposite an outlet opening in the outlet of the second container viewed in the direction of the container longitudinal axis. The second container may comprise an inlet with an inlet opening and an outlet with an outlet opening, the outlet being arranged on a side opposite the inlet. When the second container is introduced into the second receiving area by a essentially vertical movement through the open top, the second container is received by the second receiving area such that the outlet is arranged in a lower area of the second receiving area, adjoining or near to the first, lower clamping element and adjoining or near to the lower limitation. Thus, the outlet can be carried out through the through-hole in the lower limitation of the second receiving area. As the inlet is arranged in an upper region of the second receiving region, adjoining or near to the open upper side and adjoining or near to the second, upper clamping element. Since the inlet can be connected to an outlet of a fluid reservoir, fluid can be guided from the fluid reservoir into the interior of the second container and this can be done by means of the clamping elements and/or the inclined side wall fluid can be dosed in the desired amount, which is necessary for the preparation of coffee, and emerge from the second container through the outlet. This enables a predetermined or predeterminable dosage of the fluid for preparing coffee and a correct dosage of the fluid by means of the clamping elements.

Preferably, the inlet of the second container is firmly connected to the outlet of the fluid reservoir, preferably screwed or glued.

The second container can be firmly connected to the fluid reservoir. The second container and the fluid reservoir can thus be designed as a unit that is firmly connected to each other. Therefore the fluid reservoir can preferably be integrated into the container so that the fluid reservoir is formed integrally with the second container. As a result, the second container and the fluid reservoir can be introduced into the receiving area and received as a unit connected to each other. As a result of the outlet of the fluid reservoir is connected to the inlet of the second container, after the second container is received in the second receiving area, the fluid can be guided from the fluid reservoir into the interior of the second container and by means of the clamping elements in the desired amount dosed and fed through the outlet to the preparation device. This enables the fluid reservoir and the second container to be provided as a unit and the user no longer has to manually assemble the fluid reservoir and the second container. The second container can thus be connected to the fluid reservoir and form a combination container or combination bag. In this state, the combination container can be produced filled with fluid. In other words, the second container and the fluid reservoir can be filled with fluid. Here, it is conceivable here that only the fluid reservoir is filled with fluid and the second container is arranged or fastened to the fluid reservoir when folded. The fluid reservoir filled with fluid, for example a Tetra-Pak, can be separated from the second container with a separating element, for example with a clamp. The use of a clamp as a separating element prevents the fluid from flowing from the fluid reservoir into the second container and from being able to escape from the outlet of the container when the second container is opened.

However, it is also conceivable that the fluid reservoir and the second container are two separate elements which are provided separately from each other. Thus, the inlet of the second container can first be connected to the outlet of the fluid reservoir, for example by means of a screw connection or by means of a plug connection or by means of an adhesive connection or by means of a clamp connection, so that the second container and the fluid reservoir then together through the open top into the second receiving area can be introduced.

The second container can be formed from different materials and, for example, plastic or other flexible materials, for example a film material, which are suitable for receiving a fluid. Furthermore, the second container can be designed as a bag or a pouch. Like the second container, the fluid reservoir can be formed from a flexible material. However, it is also conceivable that the fluid reservoir is formed from a non-flexible material and is thus dimensionally stable, wherein the fluid reservoir can comprise a metal such as aluminum or a plastic, for example. For example, the fluid reservoir can also be designed as a cardboard box, for example a Tetra Pak. Preferably, the fluid reservoir and the second container are made from the same material, in particular if the fluid reservoir and the second container are made as one unit and not as two separate elements.

Preferably, the second container comprises a essentially horizontal plate which is arranged adjoining or near to the inlet opening and/or adjoining or near to the inlet of the second container. Preferably, the plate can be connected to the second container or the plate is firmly connected to the second container or the plate is integrated into the second container. The horizontal plate can also be integrated into the fluid reservoir.

The plate or suspension strap can be connected to the upper region of the second container in a fixed or detachable manner. The plate can be formed integrally with the second container. Preferably, the plate has a surface shape which essentially corresponds to the surface shape of a cross-section of the second container at an angle different from 0° or 180°, preferably at an angle of 90°, in particular seen transversely to the longitudinal container axis of the second container. The surface shape of the plate can be, for example, rectangular or square or circular or oval. However, other forms are also conceivable. The distance between two opposite sides of the surface shape of the cross-section of the plate is preferably equal to or greater than the distance between two opposite side surfaces of the second container when it is inserted and received in the second receiving area or when it is filled with a fluid or when inside the second container a fluid is added.

The plate enables a simplified introduction of the second container into the second receiving area and a subsequent holding or positioning of the second container in the second receiving area. When the second container is received in the second receiving area, the plate rests on the edges or marginal surfaces of the inclined side wall. In addition, the plate enables the second container to be precisely inserted into the second receiving area, so that the clamping elements can exert pressure on one of the side walls of the second container and the tempering device can come into contact with the second container. This enables precise temperature control of fluid to the desired temperature and subsequent dosing for the preparation of coffee.

As an alternative to the plate, a positioning and holding device or suspension device can be provided, which essentially fulfills a similar purpose as the plate. The positioning and holding device is preferably designed as a clamp or as a C-clamp or as a C-holding element with a C-shape. This C-clamp can be arranged between the second container and the fluid reservoir, preferably at the point at which the second container is connected to the fluid reservoir in the case of the combination container. The C-clamp can, for example, be fastened, preferably glued, to the underside of the fluid reservoir or to the top of the second container. It is also conceivable that the positioning and holding device comprises an adhesive element, for example an adhesive strip, and/or a Velcro element instead of the C-clamp or the C-holding element.

The combination container can be positioned and held by means of the positioning and holding device on one of the side walls or on the inclined side wall of the second receiving area, preferably in an upper area of the second receiving area. Due to positioning and holding device prevents the combination container, in the state inserted into the second receiving area, from sliding downwards in the direction of the lower limitation while the fluid is being emptied. This ensures that the second container can empty completely. The positioning and holding device is designed to hold the second container and/or the fluid reservoir in position.

Preferably, the plate preferably has a through-hole, wherein the plate preferably comprises a first flange with a first peripheral wall, the first peripheral wall at least partially surrounding the through-hole and extending at an angle different from 0° or 180°, in particular essentially transversely from a first Side of the plate extends away. Preferably, the first flange is designed to connect the plate to the outlet and/or to an outlet opening of the fluid reservoir.

The first peripheral wall of the first flange of the plate is designed to be able to engage with the fluid reservoir, in particular the outlet of the fluid reservoir. This enables the second container to be fluidly connected to the fluid reservoir, so that the fluid can be reliably introduced from the fluid reservoir into the second container. The first flange or the first peripheral wall can be manufactured in one piece with the plate, or manufactured as a cast part or injection-molded part that can be connected to the plate. The outer wall of the first flange can be shaped essentially round and the outer wall of the outlet of the fluid reservoir can be shaped essentially round. However, other shapes are also conceivable, for example an oval shape.

The first flange can be connected to the outlet of the fluid reservoir, for example by means of a plug-in connection. Accordingly, the inner diameter of the first flange or the first peripheral wall can essentially correspond to the outer diameter of the outlet of the fluid reservoir, or the inner diameter of the first flange or the first peripheral wall can be slightly larger than the outer diameter of the outlet of the fluid reservoir. The outlet of the fluid reservoir can thus be connected to the first flange in a simple manner, so that fluid can be reliably introduced into the second container. However, it is also conceivable that the first flange can be connected to the outlet of the fluid reservoir by a screw connection. Thus, the first peripheral wall of the first flange can comprise a first thread, for example on the inside or on the outside of the first peripheral wall relative to the through hole, which is connected to a second thread of the outlet of the fluid reservoir, for example on the outside or on the inside of a peripheral wall of the outlet, can be screwed.

Thus, the second container can thus be connected to the fluid reservoir by simply plugging or screwing it. However, it is also conceivable that the second container is glued to the outlet of the fluid reservoir by means of the first flange or that the second container is designed integrally with the fluid reservoir, for example as a combination container. A tank, which can be adapted to the dimensions of the device for preparation, can be provided as the fluid reservoir. For example, the tank can have a cross-section in the plane of one of its side faces in which the outlet and the outlet opening are arranged, which essentially corresponds to the cross-section of the device for dosing and grinding coffee beans and/or for preparing coffee, in one of 0° or 180° different angles, in particular transverse, to the longitudinal axis of the device or to the longitudinal axis of the second receiving area. However, it is also conceivable that the fluid reservoir is a bottle in which fluid suitable for the preparation of coffee is held and which can be purchased, for example, in the supermarket, for example a bottle with still mineral water. Instead of the bottle, a container, in particular a Tetra Pak, is also conceivable. In this case, the bottle opening or the opening of the Tetra Pak can be screwed to the first flange as an outlet in a simple manner, whereby, for example the external thread of the bottle opening or the opening of the Tetra Pak with the first thread, for example on the inside of the first flange or the first peripheral wall, can be screwed.

Preferably, the plate has a second flange with a second peripheral wall, the second peripheral wall at least partially surrounding the through hole and extending essentially transversely away from a second side of the plate opposite the first side. The second flange is preferably designed to connect the plate to the inlet and/or to the inlet opening of the container.

As previously described, the plate can be firmly connected to the second container, in particular a second side of the plate, which is opposite the first side with the first flange and the first peripheral wall, can be firmly connected to the second container, so that the second container by means of the plate is connectable to the fluid reservoir. However, it is also conceivable that the plate is a separate element which can be connected to the outlet of the fluid reservoir by means of the first flange and which can be connected to the inlet of the second container by means of a second flange.

The second flange comprises a second peripheral wall and is arranged on the second side of the plate in such a way that the second flange and the second peripheral wall at least partially surround the through hole. The second flange and the second peripheral wall can be configured essentially like the first flange and the first peripheral wall. Preferably, the first flange and the second flange surround the same flange center longitudinal axis, which extends at an angle different from 0° or 180°, in particular transversely to the plane of the plate and/or through the through opening. Thus, the plate can be connectable to the fluid reservoir by means of the first flange and to the inlet of the second container by means of the second flange, a fluid reservoir central longitudinal axis of the fluid reservoir and the container longitudinal axis of the second container running in the same straight line with the flange central longitudinal axis if the fluid reservoir, the plate and the second container are connected to each other. The fluid reservoir central longitudinal axis extends through the outlet, so that the outlet opening is arranged around the fluid reservoir central longitudinal axis. The longitudinal axis of the container extends through the inlet so that the inlet opening is arranged around the longitudinal axis of the container. The outlet opening and/or the outlet of the second container can also be arranged around the longitudinal axis of the container. However, it is also conceivable that the outlet opening and/or the outlet do not extend around the longitudinal axis of the container, but rather around a longitudinal axis which extends in a plane parallel to the longitudinal axis of the container.

It is also conceivable that the inlet of the second container can be connected directly to the outlet of the fluid reservoir. Thus, the plate can be dispensed with. Preferably, the outlet of the fluid reservoir can be connectable to the inlet of the second container by means of a plug connection or by means of a screw connection. The inlet of the second container can, however, also be glued to the outlet of the fluid reservoir, or can be connected to each other in one piece. Preferably, the inlet of the second container comprises the first thread, for example on the inside or on the outside of the inlet, viewed relative to the inlet opening, wherein the first thread with the second thread at the outlet of the fluid reservoir, for example on its inside or outside, viewed relative to the outlet opening, can be screwed. Preferably, the fluid reservoir comprises a housing with an upper side and a lower side, the upper side and the lower side being arranged at opposite ends to the fluid reservoir central longitudinal axis. The outlet of the fluid reservoir is arranged on the underside, the underside running extending in a essentially horizontal plane or in a plane at an angle different from 0° or 180°, in particular at an angle of 90°, viewed transversely to the fluid reservoir central longitudinal axis. With this configuration, the underside can assume the function of the plate.

The fluid reservoir can also have an inlet with an inlet opening, which is preferably arranged opposite the outlet or the outlet opening. In this way, a fluid can be introduced into the fluid reservoir through the inlet and/or added. However, it is also conceivable that the fluid reservoir does not include an inlet or an inlet opening, in particular if the combination container filled with fluid is produced.

Preferably, the outlet of the container is for insertion into a through hole configured in the lower limitation of the second receiving area.

The outlet of the second container can be designed as an elongated element, for example the outlet can be tubular and the outlet can extend between a first end and an opposite second end along a longitudinal axis of the outlet. The first end is disposed adjoining or near to the second container and the second end is spaced from the second container. The outlet has an outer diameter which is smaller than the inner diameter of the through-hole in the lower limitation of the second receiving area. Thus, the outlet of the second container can be guided through the through-hole in the lower limitation of the second receiving area when the second container is inserted into the second receiving area. This enables that, in the condition of the second container inserted in the second receiving area, the outlet can be connected to the preparation device, so that a correctly dosed amount of fluid can exit from the second container and mixed with the coffee powder also supplied to the preparation device by means of the preparation device can be and can be filled into a container, preferably into a coffee pot or cup. The first end of the outlet can be firmly connected to the second container, for example the first end of the outlet can be designed in one piece with the second container or it can be glued to the second container. The second end of the outlet, when the second container is received in the second receiving region, may lie in a plane that is below the plane of the lower limitation. However, it is also conceivable that the second end lies in the same plane as that of the lower limitation, or in a plane adjoining or near to the plane of the lower limitation, for example above the level of the lower limitation.

Preferably, the second container has at least partially a tapering section, wherein circumference of the second container in the tapering section decreasing towards the outlet, preferably essentially conically.

The second container can extend between an inlet end and an opposite outlet end along the longitudinal axis of the container. Adjoining or near to the inlet end, the inlet opening and the inlet are arranged. Adjoining or near to the outlet end, the outlet and the outlet opening are arranged. The second container can have a first side wall and an opposing second side wall which extend essentially parallel to the plane of the longitudinal axis of the container between the inlet end and the outlet end. In the lower area near to the outlet end, the container has a tapering section. In the tapering section, the distance between the first side wall and the second side wall decreases towards the outlet, preferably essentially conically. This enables the fluid to be guided almost completely out of the second container through the outlet, so that a minimal amount of residual fluid remains in the second container.

Preferably, the second container has at least partially a essentially symmetrical section, wherein the periphery of the second container remaining the same within the essentially symmetrical section and wherein the essentially symmetrical section is spaced further from the outlet than the tapered section.

In the essentially symmetrical section, the first side wall and the second side wall each extend in a plane parallel to the plane of the longitudinal axis of the container. The essentially symmetrical section may extend between the inlet end and the tapered section. In the state when the second container is inserted or received in the second receiving region, the clamping elements of the individual pairs of clamping elements are adjoined to the side walls in the essentially symmetrical section and can apply pressure to the side walls. This enables the fluid for preparing coffee to be dosed. Preferably, the first, lower pair of clamping elements is preferably arranged on the side walls of the second receiving area in such a way that it can exert pressure in an area of the second container which lies within the essentially symmetrical section and is adjoining or near to the tapering section.

Preferably, the second container comprises at least one first magnet, wherein the at least one first magnet is preferably arranged on an outer wall of the tapered portion, and wherein the at least one first magnet with at least one second magnet adjoining or near to the through hole in the lower limitation of the second receiving area is connectable.

The at least one first magnet can be arranged adjoining or near to the outlet, preferably on an outer wall of the outlet. The at least one second magnet can be arranged adjoined or near to the through hole in the lower limitation, preferably on an inner wall of the through-hole. The at least one first magnet can at least partially surround the outer wall of the outlet, preferably the at least one first magnet can completely surround the outer wall of the outlet. The at least one second magnet can at least partially surround the inner wall of the through-hole, preferably the at least one second magnet can completely surround the inner wall of the outlet. Preferably, the at least one first magnet and the at least one second magnet are arranged in such a way that they can interact when the second container is received in the second receiving area. In this way, the second container is brought or held in a suitable position so that the fluid can be guided almost completely out of the second container through the outlet and/or so that the individual clamping elements enable or ensure optimal dosing of the fluid. Alternatively, it is also conceivable that, instead of the at least one first magnet, a metal element (or a metal plate or a metal strip) is provided which interacts with the second magnet. It is further conceivable that instead of the at least one second magnet, a metal element (or a metal plate or a metal strip) is provided which interacts with the first magnet. Due to the magnets, the second container is always in the correct position so that the fluid for preparing coffee can be guided out of the outlet without fluid reaching the housing of the second receiving area.

Preferably, the second container is a hose or the second container is designed as a hose.

Preferably, the fluid in the interior of the second container can be dosed by means of a peristaltic pump.

Preferably, the second container or the hose and/or the peristaltic pump can preferably be introduced into the second receiving area of the device for dosing and grinding coffee beans and/or for preparing coffee and can be received by the second receiving area.

Preferably, the hose and/or the peristaltic pump and/or the fluid reservoir are p exchangeable and designed as single-use or disposable article.

Preferably, the hose and the fluid reservoir are interconnectable or interconnected.

Preferably, a tempering device, for example a heating plate and/or a cooling plate, is arranged adjoining to or near to the fluid reservoir. Further preferably, the tempering device is preferably in contact with the fluid reservoir.

Preferably, at least one clamping element is arranged adjoining or near to the fluid reservoir. Preferably, the at least one clamping element is designed as a clamp. Preferably, the at least one clamping element or the clamp is designed to heat and/or cool at least part of the fluid in the interior of the fluid reservoir.

Preferably, the first container and/or the second container and/or the dosing device or the screw conveyor and/or the hose and/or the peristaltic pump are made from a bioplastic or bioplastic or a bio-based plastic. Preferably, the first container and/or the second container and/or the dosing device or the screw conveyor and/or the hose and/or the peristaltic pump comprise a bioplastic or bioplastic or a bio-based plastic. For example, the bioplastic can comprise stone paper and/or wood.

Preferably, the first container, for example after emptying the coffee beans or after reaching a certain fill level, and/or the second container, for example after emptying the fluid or after reaching a certain fil level, can be automatically ordered on the Internet.

Preferably, the sensor or the scale is connected to application software, for example a mobile app, so that the level of the fluid can be automatically indicated, for example by a signal tone or a signal light, so that a new container with fluid or a new container with coffee beans can be prepared manually and/or so that a new container with fluid or a new container with coffee beans can be automatically ordered on the Internet.

Preferably, the device or the second container comprises a positioning and holding device which is designed to position and hold the second container in the second receiving area.

Preferably, the device as previously described can be operated remotely. The device can be regulated or controlled from anywhere and at any time, for example using an app on the smartphone or computer or remote control. In this way, the coffee can be prepared remotely without someone having to be on site near the device. Furthermore, different operating schedules are conceivable so that the device can automatically prepare a coffee at a predetermined point in time.

It is conceivable that a so-called community coffee can be prepared. Community coffee means that a group of people can access the device, for example by means of an app on their smartphone or by computer or by remote control, in order to place an order for a coffee to be prepared. For this purpose, a computer-implemented method for controlling or regulating the device described above (and for preparing a community coffee) is conceivable, which comprises at least one of the following steps:
Recognize that someone is preparing or wanting to prepare a certain type of coffee in a certain period of time; and or
Informing, for example by a signal tone or a pop-up window on the screen of a smart device or smartphone or computer or a text message (SMS) that someone is preparing or would like to prepare a certain type of coffee within a certain period of time and or
Ordering, for example using a book button on the screen of the computer or smartphone, a desired number of cups of coffee and or
Information approximately the maximum volume or the maximum number of cups that can be booked. This means that there are more pre-orders than there is space in a container, for example in a coffee pot, so another community coffee has to be made and the user is informed that his order will be taken into account for the next preparation (the process can be repeated); and or
Setting, for example via app, for which period a pre-order should exist and whether automatic preparation should take place or not; and or
Preparation of a coffee including all orders. Now, if someone makes a coffee, the coffee plus the bookings or orders are prepared; and or
Inform, for example by means of a signal tone or a pop-up window on the screen or a text message (SMS), that the coffee is ready. After a coffee has been prepared, all participants who have ordered or booked a coffee can be informed that the coffee is ready. Furthermore, information can also be provided as to whether an order has been taken into account or when it will be taken into account, e.g. with the next preparation; and or
Confirmation by the user, for example via the screen, that the coffee has been taken or picked up; and or
Notification, for example by means of a signal tone or a pop-up window on the screen or a text message (SMS), that a new use or a new community coffee is intended. For example, it can then be agreed, verbally or via app, that normal filter coffee should be prepared so that a filter should be inserted into the device and a coffee pot should be placed accordingly.

The computer-implemented method as previously described enables a defined amount of coffee to be prepared, precisely as much as one variety of people is desired. This prevents too much coffee from being prepared that is not drunk in the end.

The computer-implemented method as previously described can be used in a corporate office, but also in a cafe or in a coffee shop. The process can also be used for other types of preparation such as cold brew, cold drip or espresso. Remote pre-orders are also conceivable, for example early in the morning when someone is on their way to work so that they can have their coffee as soon as they arrive.

If automatic preparation is not to take place, the following sequence is possible: When enough orders have come together in a predetermined period of time, a person at the machine is asked to prepare the system. Alternatively, someone from the community or in the college is selected for this by the software. The selection takes into account who was there and how often. Thus, no participant is disadvantaged.

By means of a text message, for example SMS, or a signal tone or a pop-up window on the screen of the computer or smartphone, the chosen person can be informed that he or she should carry out the preparation. It is also conceivable to have a billing system in which it is determined via app which participant has placed an order and how often. It is also conceivable that the device automatically recognizes which participant is approaching the device and thus makes an automatic booking. This can be done, for example, via smartphone recognition or via recognition of a key with an RFID chip. Preparation can therefore only be possible if a participant has their smartphone or key with an RFID chip with them. Recognition by manually entering an identification code on an input field of the device is also conceivable.

Another computer-implemented method for controlling or regulating the device as previously described can comprise the following steps: Dosing the coffee beans from the first container using the dosing and grinding device and/or dosing the fluid from the second container using a further dosing device, for example using the clamping elements as previously described or the lifting system described above, and/or preparing the coffee using the preparation device (as previously described or below), and/or determination of the level in the first container, which is designed to hold coffee beans, and/or determination of the level in the second container, which is designed to hold a fluid, and/or identification the first component and/or the fluid and/or re-ordering the coffee beans and/or the fluid based on the determined level.

Preferably, the device recognizes the type of preparation device in an automated manner.

It is conceivable that the device can recognize the preparation device that is inserted in the device via a sensor element, for example via a click sensor. Ideally, the device or the sensor element can detect whether a coffee pot or a coffee cup is arranged in the device. Preferably, the device detects whether the coffee pot or the coffee cup is suitable for receiving the amount of coffee to be prepared. Furthermore, it is also conceivable that the device comprises a barcode reader with which a barcode, which can be attached to the dosing and grinding device, for example, can be read. The barcode can contain data approximately the type of bean and the grinder.

Furthermore, depending on the recognized preparation device, the device can take into account certain preparation instructions and sequences. Preferably, the device is designed to check whether the appropriate coffee and the appropriate grinder are used for the selected type of preparation. It is conceivable that the device can output a signal tone, so that it is indicated that an unsuitable coffee or an unsuitable grinder is being used.

Preferably, the preparation devices (or preparation units) have at least one ring. The at least one ring can have at least one notch. This enables the device to use the number of rings and/or the number of notches to determine the respective preparation device or type of preparation, e.g. filter coffee, cold brew, cold drip, espresso, Karlsbader, etc., can recognize. Alternatively, it is conceivable that the type of preparation device can be recognized via RFID, barcode or different ring sizes.

The device is thus designed to accommodate the various preparation devices and to recognize the type of preparation device on the basis of the number or type of rings and/or the notches. The preparation devices can thus be integrated or used in the device and recognized or identified by means of the sensor element. In this way, the device can automatically prepare the desired coffee or the desired type of coffee, depending on the preparation device used. However, it would also be conceivable that additional settings with regard to the type of preparation can be selected or adjusted, for example via a control panel.

The device is designed to accommodate a holding element for the preparation device, for example a holder for a paper filter or a holder consisting of a housing with a filter. The holding element can be made of porcelain or at least partially comprise porcelain, for example on the inner surfaces which come into contact with the preparation device. The filter can also be made of porcelain or at least partially comprise porcelain. It is conceivable that the holding element at least partially comprises glass, metal and/or plastic, or that the holding element is made of glass, metal or plastic. The coffee pot or barrel can be positioned under the filter. The coffee pot can be combined or connected to the various filters or filter holders.

Preferably, there is for each type of preparation, such as filter coffee, cold brew, cold drip or espresso, there is a separate preparation device that can be automatically recognized by the device or by the sensor element.

It is conceivable that the preparation devices can be connected or combined with a coffee pot or a coffee cup. The preparation device can comprise a water receiver, a portafilter and a further attachment. The device can be designed so that temperature-controlled or preheated water is first filled into the water receiver up to just below a valve. Then the portafilter or filter can be clicked or hung into the device to fill it with espresso powder (freshly ground). The device can automatically provide the correct proportions of fluid or water and powder.

It is also conceivable that the preparation device, for example the preparation device for the preparation of espresso, can be positioned below the device, preferably adjoining or near, or can be clicked into the device, preferably at a lower position of the device. Then only a cover or attachment needs to be put on. In other words, separate components such as a water receiver, a portafilter and another attachment can be dispensed with. The device can comprise a tempering field or a hob which can be used for a milk foamer and for the preparation device, for example for the espresso preparation device. The tempering field can be designed for heating but also for cooling.

When the water and the coffee powder are mixed or come into contact with each other, for example in the portafilter or filter, the water can rise, which is accompanied by a bubbling-hissing sound. The device can be designed, for example, comprise a temperature sensor, in order to determine the temperature of the coffee powder/water mixture. In addition, the effect of the bubbling-hissing noise or the water flowing up can be taken into account, for example by a determination unit to determine when the coffee or espresso is ready, so that the corresponding preparation device can be removed from the device. The device can comprise a heating plate and to control or regulate the temperature.

Correspondingly, when preparing by means of the cold-drip type of preparation, a corresponding preparation device can be hooked into the device, which device can also be recognized by the device or the corresponding sensor element. A clamping element can be provided, for example a clamp, which can hold the filter and the jug. Furthermore, a cooling water sensor can be hung in the device. This can also be placed under the device. As soon as the device has determined the corresponding preparation device, for example using the click sensor, the corresponding type of preparation, here cold drip, is carried out.

The device can have a control panel which is displaceable in order to use the device transversely but also I longitudinally.

Preferably, a system is provided, the system comprising a device for dosing and grinding coffee beans and/or for preparing coffee, a first container for receiving and dosing and grinding coffee beans, and/or a second container for receiving and dosing fluid for making coffee. The device provided in the system for dosing and grinding coffee beans and/or for preparing coffee can have all of the features described above and have the advantages associated with these features. The first container for receiving and dosing and grinding coffee beans can have all of the features described above and have the advantages associated with these features. The second container for receiving and dosing fluid for preparing coffee can have all of the features as previously described and have the advantages associated with these features.

The container or the filter container can be part of the device for dosing and grinding coffee beans and/or for preparing coffee and can be arranged so that the ground coffee powder and the fluid can be introduced into the container, preferably by gravity. The container or the filter container can preferably be arranged below the through-hole through which the outlet of the screw conveyor housing and the outlet of the second container can be guided.

Preferably, the device for dosing and grinding coffee beans and/or for preparing coffee preferably has a rotatable or rotating plate or a rotatable or rotating heating plate. With rotatable or rotating is meant that this plate or this heating plate can be set in a rotating movement or in a circular movement. The rotating heating plate is preferably arranged below the through-hole through which the outlet of the screw conveyor housing and the outlet of the second container are guided. The container or the filter container can be arranged on this rotating plate and can be held in a fixed position on the rotating plate by lateral limiting elements or position elements.

The water or the fluid can be applied to the coffee powder in a circular motion in order to achieve even moistening. This can be done by the rotating plate. It is also conceivable that in this way a uniform filling of the filter container with coffee powder can be supported. In this case, the filter container is connected to the jug or cup. Alternatively, a kind of spinning top can be attached directly to the filter container or to the filter holder (brewing group). The gyroscope can have the shape of a horizontal propeller. When the water or fluid hits the blade, it rotates. This rotates the point at which the water or fluid meets the coffee powder. In this way, a circular movement and thus a uniform application of the water or fluid can be achieved. The blades can correspond to the blades on the propeller. The screw is set in rotation by the impacting water. This distributes the water evenly over the coffee powder. The gyroscope can comprise a plurality of wings, preferably the gyrosope comprises at least two wings, preferably three wings, more preferably five wings.

For example, a coffee pot can be connected to the filter container, and this coffee pot is arranged on the rotating plate. In this way, the coffee powder in the filter container can be moistened uniformly with the desired amount of fluid, while the filter container is set in a rotating movement, so that the coffee powder can be mixed with the fluid particularly well. In an advantageous manner, the entire coffee powder is mixed with fluid or soaked through and as little coffee powder as possible can enter the coffee pot through the filter. This improves the quality of the coffee. The rotating plate means there is no need for nozzles. Alternatively, it is conceivable that the mixing of the coffee powder with the fluid takes place via 3D acoustic waves or via the preparation device. The mixing can be used in a cold brew type of preparation or in a dripping type of preparation. It is advantageous if a uniform soaking or softening of coffee powder can be ensured with fluid, which can be done, for example, by the propeller as previously described. The mixing can also be done manually by swiveling motion and/or by stirring with a spoon.

Preferably, the device for dosing and grinding coffee beans and/or for preparing coffee comprises a timer or a camera which are designed to detect the swelling or bloom of the coffee or the coffee powder. Approximately 30 seconds after the coffee powder with the fluid has been placed in the filter container, the coffee begins to swell (so-called “bloom”). This causes carbon dioxide (CO2) gases to escape from the coffee. The coffee becomes heavier and adheres more to the filter. This promotes a uniform extraction. This is essential for a good coffee. The swelling or bloom can be adjusted or detected by means of the timer or the camera, so that a further introduction of fluid from the second container can take place. The further dosing of the fluid can take place via the clamps in the second receiving area. A regulating or control device can be provided by means of which the clamps can be controlled or regulated for further dosing of the fluid. Thus, the clamps can be used for a first dosage of fluid and, once the coffee has swelled, a second dosage of fluid can take place by means of the clamps. The clamps can, however, also be controllable or controllable manually by an operator.

The regulating or control device can also be used to regulate or control the filling speed of the filter container with coffee powder and/or fluid. For example, the filling speed can be regulated or controlled by means of the hydrostatic pressure of the fluid. This regulation or control can take place via different positions of the clamps, so that different filling quantities can be set in the second container, which generate different hydrostatic pressure and thus lead to different flow speeds. Adjustments can be made using one of the brackets if necessary. However, it would also be conceivable that the slope of the inclined side wall and the plane in which the three brackets extend can be changed or adjusted relative to the lower limitation. This can be done either automatically by the regulating or control device, or manually by an operator. Depending on the slope of the inclined side wall and the plane, the flow rate of the fluid can thus be determined or influenced.

The previously described device for dosing and grinding coffee beans and/or for preparing coffee can be used, for example, as a coffee machine that can prepare the coffee using the dripping process, in which cold water gradually, drop by drop, through a paper filter in the filter container and onto the coffee powder so that iced coffee collects in the glass or coffee pot below. It should be noted that the paper filter described above is optional. For this purpose, an attachment can be arranged at the outlet of the second container. The attachment can be connectable to the outlet, it can be glued or screwed to the attachment. The attachment can be designed as a valve in order to adjust the dripping speed of the fluid from the second container. For example, every two seconds a drop could be introduced from the second container into a filter container, which is preferably arranged below the through-hole and thus below the outlet. A coffee pot or a glass pot is preferably arranged below the filter container as previously described. The device is designed to enable a suitable dosing of fluid and coffee powder. The coffee powder is moistened (must be dosed in such a way that all of the powder is moist) using the machine (followed by the attachment). The powder can be mixed via 3D acoustic waves or via the preparation device or via the shaking function or shaking device or via the rotating plate on which the filter container and/or the coffee pot are arranged. Then a paper filter can be placed on the coffee powder in the filter container. This is done manually. Preferably, the machine can give a signal when the paper filter is to be placed or placed on the coffee powder. The sensor element or the camera element can be used to determine when the correct mixture has been achieved. The fluid can be cooled, for example, by means of the tempering device, which can be designed as a combined heating and cooling plate. However, it is also conceivable that ice cubes are placed in the water reservoir or that the water reservoir comprises ice cubes. The heating element can then be switched off accordingly. It is conceivable that the attached valve is automatically controlled or regulated. The machine can regulate or control the dripping speed via a camera or sensor element. The dripping speed can also be controlled or regulated via the hydrostatic pressure.

The device for dosing and grinding coffee beans and/or for preparing coffee can also be used for other types of preparation, for example Chemex or French Press or Cafe Solo Brewer or hand filter or Karlsbader Kanne or AeroPress. The required quantities of coffee powder and/or fluid, as well as the degree of grinding and the length of time in which the fluid and the coffee powder are in contact, can be regulated or controlled by means of the regulating or control device and/or the sensor elements or camera elements. The following table shows the preferred values for the selected preparation method:

| Type of | coffee | Water | Recommended | |
|---|---|---|---|---|
| preparation | amount | amount | grinding degree | Contact time |
| Chemex | 18 g | 148 ml | medium coarse | approx. 4 min |
| Gold Brew | 50 g | 237 ml | very coarse | approx. 12 h |
| French Press | 10 g | 155 ml | Rough | approx. 3-4 min |
| Cafe Solo | 18 g | 113 ml | medium coarse | approx. 4 min |
| Brewer | | | | |
| Hand filter | 16 g | 250 ml | medium fine | approx. 3-4 min |
| Carlsbad jug | 22 g | 350 ml | very coarse | approx. 4-5 min |
| AeroPress | 16 g | 200 ml | medium fine | approx. 30 s |

It is conceivable that the device comprises a memory unit in which the values shown in the table are stored. For example, the contact time can be determined automatically based on the desired type of preparation, in which the regulating or control unit can access this table. The required preparation time can be transferred to the control unit using the app.

Preferably, a device for dosing and/or preparing baby food, in particular baby milk or baby food, or coffee is preferably provided, comprising: a housing with a first receiving area and a second receiving area, wherein the first receiving area being designed to accommodate a first container for baby food concentrate or coffee powder and wherein the second receiving area is designed to receive a second container for a fluid (in particular liquid), a tempering device for tempering of the fluid, and a dosing device for dosing the baby food concentrate or the coffee powder. The first receiving area has a dosing device receiving area for receiving the dosing device and an actuating and/or drive device for the dosing device is arranged in the dosing device receiving area.

The device has a first receiving area which is designed to receive a first container with baby food concentrate or coffee powder. Furthermore, a dosing device receiving area is arranged in the first receiving area of the device, i.e. a receiving area in which a dosing device can be received. Thus, the first container with baby food concentrate or coffee powder and the dosing device can be at least partially received in the first receiving area. This advantageously enables the dosing device to interact with the first container. In particular, correct dosing of the baby food concentrate or the coffee powder can be carried out by the dosing device. This is made possible by the fact that the dosing device is driven by the drive device which is also arranged in the dosing device receiving area.

Furthermore, all components of the device described that come into contact with the baby food concentrate or with the coffee powder or with the fluid are in particular exchangeable and can be easily removed from the device. By exchangeable components, it means that the components are designed as disposable or single-use-articles. In particular, the first container for baby food concentrate or coffee powder, the dosing device for dosing the baby food concentrate or coffee powder and the second container for a fluid are interchangeable. The first container can be connected or fluidly connected to the dosing device and/or the second container can be connected or fluidly connected to a fluid reservoir. This is advantageous because the device for preparing baby food, in particular the first receiving area and the second receiving area, does not come into contact with the baby food concentrate or coffee powder and the fluid. Thus, the device, in particular the first receiving area and the second receiving area, is not contaminated with baby food concentrate or coffee powder and fluid, so that cleaning of the device is not necessary after each individual preparation of baby food or coffee. Furthermore, the device and/or its individual components do not need to be decalcified.

The baby food concentrate or coffee powder and the fluid can be introduced through the device into a container, preferably into a baby bottle or into a coffee cup, in the correct mixing ratio. Thus, the baby food or the coffee can be mixed and prepared ready to drink by shaking or jiggling the container or the baby bottle or the coffee container or the coffee cup.

However, it is also conceivable that the device comprise a preparation device for preparing baby food or coffee from the baby food concentrate or coffee powder and the fluid, which can also be exchangeable and designed as a disposable or single-use-articles. With the device, the baby food concentrate or coffee powder from the first container and the fluid (e.g. a liquid) from the second container can be fed to the preparation device and introduced into a funnel and/or filter container in the correct mixing ratio, so that the coffee is in a further, separate container, in particular in a baby bottle or in a coffee cup, can be introduced. This enables the baby food or coffee to be prepared correctly. Thus, the device can be designed to recognize the configuration, for example the shape and/or the volume and/or the size, of the further, separate container and the filling of the further, separate container with fluid and/or baby food concentrate or coffee powder based on to make its design automated.

The tempering device can bring the temperature of the fluid in the second container to the preparation temperature provided or recommended by the manufacturer of the baby food concentrate or the coffee powder. Furthermore, the tempering device enables e.g. a sterilization of the fluid before the fluid is supplied to the preparation device. Sterilization is particularly beneficial when preparing baby food. This can be done, for example, by first bringing the fluid to or near the boiling point in order to kill germs and bacteria in the fluid. The fluid can then be tempered to the desired temperature, for example by cooling. The fluid can, however, also be tempered by heating it to the desired temperature, if the fluid has already cooled below the intended drinking temperature. In this way, the fluid can be kept at the correct temperature in order to be administered sterile to the baby. The device thus enables a simplified and safe preparation of baby food.

Preferably, the temperature control device is controllable or regulatable. For this purpose, the device can comprise a control or regulating unit. It is conceivable that the tempering device is designed as a heating plate and/or a cooling plate, or that the tempering device comprises at least one heating plate and/or at least one cooling plate. It is also conceivable that different zones or areas of the tempering device or the heating plate and/or the cooling plate are controllable or regulatable or can be activated.

Preferably, the dosing device can be connected to the first container.

The dosing device can be connectable to the first container. This means that the dosing device can be connected to the first container so that the dosing device and the first container can be introduced into the receiving area and/or removed again together. For example, the dosing device can be firmly connected to the first container (e.g. glued and/or welded) so that the dosing device and the first container are firmly connected to each other. However, it is also conceivable that the dosing device and the first container are detachably connected to each other.

This enables the first container and the dosing device to be introduced into the first receiving area in a simplified manner and at least partially received by the first receiving area. At the same time, the dosing device can safely intervene with the drive device so that the intended quantity of baby food concentrate or coffee powder can be fed from the first container into the preparation device by means of the dosing device. However, it is also conceivable that the dosing device and the first container are not connected to each other and are introduced into and/or removed from the first receiving area separately from each other.

Preferably, the dosing device comprises a screw conveyor and a screw conveyor housing, wherein the screw conveyor, preferably in its full length, is introduced into the screw conveyor housing and/or rotatably arranged or supported therein, so that the screw conveyor and the screw conveyor housing extend around a common longitudinal axis of the conveyor screw axis.

The dosing device can be designed as a screw conveyor with a screw conveyor and a screw conveyor housing. The screw conveyor can be designed as a shaft, coiled around one or more helically wound flights in the form of flat metal sheets and/or rubber flaps or wings, which essentially extend in the form of a screw thread transversely away from the longitudinal axis of the conveyor screw. Preferably, the conveyor screw is designed as a rigid conveyor screw. However, it is also conceivable that the screw conveyor is designed as a flexible, in particular bendable screw. The screw thread can either be firmly connected to the shaft, for example welded, or it can be manufactured or manufactured in one part with the shaft. This preferably screw conveyor comprises a continuous and continuous screw thread which extends between the opposite ends of the screw conveyor along the longitudinal axis of the screw conveyor enables, in particular, baby food concentrate or coffee powder to be transported by means of the conveyor screw along its longitudinal axis. The screw conveyor, in particular the screw thread, can be turned from a solid material, for example from a piece of round steel, or manufactured as a cast part or injection-molded part. The screw conveyor and/or the screw conveyor housing are essentially cylindrical in shape.

The design of the dosing device enables the baby food concentrate or coffee powder to be fed from the first container into the dosing device and transported by means of the screw conveyor in the screw conveyor housing along the longitudinal axis of the screw conveyor. With each turn of the screw conveyor, a certain amount of powder can be conveyed so that the dosage of baby food concentrate or coffee powder can be determined by the number of (partial) rotations. This enables precise and simplified dosing of the baby food concentrate or the coffee powder, which can take place both automatically, for example controlled by a regulating or control device, or manually.

Preferably, the screw conveyor housing has an inlet with an inlet opening and an outlet with an outlet opening. The inlet and the outlet are preferably arranged in the screw conveyor housing on opposite sides, viewed transversely to the longitudinal axis of the screw conveyor.

Through the inlet opening into the inlet, baby food concentrate or coffee powder can be fed from the first container into the interior of the screw conveyor housing in order to be taken up by one or more helically wound flights of the screw conveyor. The device for preparing baby food or coffee can comprise a shaking device with which the first container or its contents can be set in a shaking movement. This enables the baby food concentrate or coffee powder to be guided almost completely out of the first container through the inlet opening into the inside of the screw conveyor housing, especially if the baby food concentrate or coffee powder does not slip by itself and is to be guided into the inside of the screw conveyor housing, for example by gravity. Preferably, the shaking device can be arranged in or corresponding to the first receiving area.

As a result of the rotation of the conveyor screw, the baby food concentrate or coffee powder is conveyed by the conveyor screw essentially along the conveyor screw's longitudinal axis after entering the interior of the conveyor screw conveyor housing and can exit through the outlet opening of the outlet. Through it the outlet is arranged on an opposite side of the inlet, viewed transversely to the longitudinal axis of the conveyor screw, the baby food concentrate or coffee powder can exit the conveyor screw conveyor housing when it reaches the inlet.

Preferably, the screw conveyor has a screw flank diameter, i.e. an outer diameter transverse to the longitudinal direction of the screw conveyor, which is in a range of approximately 20 to 40 mm. Particularly, preferably, the screw flank diameter is particularly preferably approximately 25 mm. This dimensioning of the screw flank diameter favors the conveyance or dosage of the baby food concentrate or the coffee powder. The properties of the baby food concentrate or the coffee powder can change significantly as a result of moisture in particular, especially if the baby food concentrate or coffee powder (partially) clumps together or sticks together. The previously described dimensioning of the screw flank diameter ensures that the baby food concentrate or coffee powder is conveyed and dosing correctly even if moisture penetrates.

Preferably, the conveyor screw has a length which lies in a range between approximately 60 and 120 mm. Particularly, preferably, the length of the screw conveyor is between approximately 90 mm and 110 mm, further preferably approximately 106 mm. This dimensioning of the length of the screw conveyor favors the conveyance of the baby food concentrate or the coffee powder. If the length of the screw conveyor is reduced, the baby food concentrate or coffee powder can form bridges in the one or more helical passages, so that the inlet opening is blocked and no further baby food concentrate or coffee powder can be introduced through the inlet opening. The bridging can occur especially when the baby food concentrate or coffee powder is to be fed through the inlet opening into the screw conveyor housing by means of gravity.

A dimensioning of the length and the screw flank diameter of the screw conveyor in the value ranges as previously described enables a delivery rate of baby food concentrate or coffee powder in the range of approximately 5 to 10 g per revolution of the screw conveyor (e.g. approximately 8.8 g per revolution). The number of revolutions (or the angle of rotation around the longitudinal axis) allows the desired amount of baby food concentrate or coffee powder to be fed through the outlet of the screw conveyor housing and thus out of the screw conveyor housing. This enables precise dosing of the baby food concentrate or the coffee powder for the preparation of the baby food.

Preferably, the inlet opening is essentially oval-shaped and extends in the direction of the longitudinal axis. However, other shapes of the inlet opening are also conceivable. The inlet opening has a length in the range from approximately 20 mm to 60 mm (e.g. from approximately 47 mm) in the direction of the longitudinal axis of the conveyor screw and/or a length in the range from approximately 10 mm to 40 mm (e.g. from approximately 29 mm) transversely to the longitudinal axis of the conveyor screw, in particular seen perpendicular to the longitudinal axis of the screw conveyor. Preferably, the outlet opening is essentially rectangular and extends in the direction of the longitudinal axis. However, other shapes of the outlet opening are also conceivable. The outlet opening comprises a length in the range from approximately 20 mm to 50 mm (e.g. from approximately 30 mm) in the direction of the longitudinal axis of the conveyor screw and/or a length in the range from approximately 5 mm to 20 mm (e.g. from approximately 10 mm) across the longitudinal axis, in particular seen perpendicular to the longitudinal axis of the screw conveyor. These dimensions of the inlet opening and outlet opening enable a particularly favorable introduction and execution of baby food concentrate or coffee powder in the screw conveyor housing.

Preferably, the screw conveyor housing extends between a first end and an opposite second end along the longitudinal axis of the screw conveyor, the outlet being arranged adjoining or near to the first end and the inlet being arranged adjoining or near to the second end.

The inlet and the outlet are preferably arranged at a distance from each other in the longitudinal direction. By arranging the inlet adjoining or near to the second end and arranging the outlet adjoining or near to the first end of the screw conveyor housing, the baby food concentrate or coffee powder can, after entering the interior of the screw conveyor housing through the inlet opening in the inlet of one or more helically wound flights are taken up and conveyed by the rotation of the screw conveyor to the second end of the screw conveyor housing and exit again through the outlet opening. Thus, a predetermined or predeterminable amount of baby food concentrate or coffee powder can be conveyed per revolution, so that a dosage can be set (or controlled or regulated) based on the number of revolutions (or the angle of rotation around the longitudinal axis).

The first end of the screw conveyor housing is preferably designed to be open and the second end of the screw conveyor housing is preferably designed to be closed. Thus, the screw conveyor can be completely inserted into the screw conveyor housing through the first end. An insertion element or a removal element, which extends away from the second end, can be provided at the second end. The insertion element or removal element can be designed as a tab comprising a surface that is approximately the size of a thumb. In particular, the insertion element or removal element can have a length of approximately 3 to 4 cm and/or a width of approximately 2 to 3 cm. On opposite sides, the insertion element or removal element can comprise a haptic corrugated structure. Preferably, the corrugated structure is made of a soft, rubberized material. However, it can also be made of the same material as the insertion element or removal element.

By means of dosing device can be held and/or introduced into the dosing device receptacle in a targeted manner by means of the insertion element. Furthermore, the dosing device can also be easily removed again by means of the insertion element, in particular if the first container is empty and has to be replaced.

Preferably, the inlet comprises a flange with a peripheral wall which at least partially surrounds the inlet opening and extends (preferably essentially radially) away from the screw conveyor housing, the flange for connecting the dosing device to the first container and/or for introducing the dosing device into the dosing device receiving area is designed.

The peripheral wall of the inlet in the screw conveyor housing is designed to be able to engage with the first container, in particular with an outlet in the first container. This enables the baby food concentrate or coffee powder to be introduced into the screw conveyor housing particularly reliably from the first container. The peripheral wall can be manufactured in one piece with the screw conveyor housing, or it can be manufactured as a cast part or injection-molded part that can be connected to the screw conveyor housing.

The peripheral wall can extend away from the edge of the inlet opening in the screw conveyor housing essentially at an angle different from 0° or 180°, in particular transversely. The peripheral wall, like the inlet opening, can thus be essentially oval-shaped and extend in the same direction as the longitudinal axis of the conveyor screw. However, other shapes are also conceivable for the peripheral wall. In particular, the peripheral wall has essentially the same shape as the inlet opening. The peripheral wall can have a circumference in the range of approximately 100 mm to 130 mm (e.g., approximately 122 mm). The peripheral wall can extend along a first peripheral wall central longitudinal axis, which can have a length in the range of approximately 30 mm to 60 mm (e.g., approximately 47 mm). Furthermore, the peripheral wall can extend along a second peripheral wall central longitudinal axis, which is oriented perpendicular to the first peripheral wall central longitudinal axis, and/or can have a length in the range from approximately 20 mm to 40 mm (e.g. from approximately 29 mm). Other lengths are also possible. The length of the first peripheral wall part longitudinal axis is preferably greater than the length of the second peripheral wall central longitudinal axis. The lengths of the first and second peripheral wall central longitudinal axes described above are particularly favorable for introducing the baby food concentrate or the coffee powder into the screw conveyor housing and/or for connecting the dosing device to the first container.

Preferably, the peripheral wall preferably comprises a first contact surface and an opposing second contact surface, the first and second contact surfaces being aligned parallel to each other.

The first and second contact surfaces can be arranged on opposite sides of the second peripheral wall central longitudinal axis. These contact surfaces enable a particularly simple introduction of the dosing device into the dosing device receiving area. In particular during the introduction into the dosing device receiving area, the contact surfaces can slide along lateral guide elements in the first receiving area and, after being received in the dosing device receiving area, can rest against the lateral guide elements. The first contact surface and the second contact surface can have an essentially parabolic cross-sectional area. Due to the design of the two contact surfaces and the lateral guide elements, as well as their interaction when the first container is inserted into the first receiving area, the first container can be received in a correct position by the first receiving area, so that the baby food concentrate or coffee powder comes out in the correct dosage can be guided to the outlet of the dosing device.

Preferably, a coupling device extends from a drive end of the screw conveyor in the direction of the longitudinal axis, the coupling device being designed to interact, in particular to intervene, in a coupling manner with the actuating and/or drive device.

The coupling device can be designed as an essentially cylindrical cavity and/or as a receptacle, so that after the dosing device has been introduced and received in the dosing device receiving area, a coupling element in the dosing device receiving area can simultaneously be received in the (preferably essentially cylindrical) cavity. The inner wall of the (cylindrical) cavity preferably has an inner profile which can be brought into engagement with an outer profile of the outer wall of the coupling element. For example, the outer profile of the coupling element can have at least one material elevation which can engage or interact with at least one material recess in the inner profile of the cylindrical cavity. The coupling element can be designed as a drive shaft, so that the introduction of the coupling element into the cylindrical cavity enables the dosing device to be driven and thus the screw conveyor to rotate. Preferably, the transmission ratio of the rotational speed is adjustable or variable. This enables the speed of the baby food concentrate conveyed through the screw conveyor housing to be changed and thus a change in the dosage of the baby food concentrate.

Preferably, the screw conveyor housing comprises an outer wall with a plurality of ribs, wherein the ribs preferably extend essentially in the axial direction at least partially between the first end and the second end, and/or wherein the ribs essentially in the radial direction away from the outer wall extend

The ribs are preferably as longitudinal ribs between the first and second end formed and/or surround the outer wall in the peripheral direction at regular or symmetrical intervals. The ribs may extend away from the outer wall so that each of the ribs has an outer edge that runs in a straight line that is essentially parallel to the longitudinal axis of the screw conveyor housing runs and/or a essentially constant distance to the outer wall of the has conveyor screw conveyor housing. The ribs can, however, also have a e.g. have conically shaped area which is preferably near to the first end of the screw conveyor housing. In this conically shaped area, the outer edge of the ribs tapers towards the first end of the screw conveyor housing.

Preferably, two further ribs limit the outlet opening on or on opposite sides in the peripheral direction of the outer wall. In other words, two of the ribs are arranged adjoining or near to the outlet opening and extend away from the edge of the outlet opening. Preferably two further ribs are provided which limit the outlet opening on opposite sides in the axial direction of the outer wall. These further ribs run between the two ribs delimiting the outlet opening on opposite sides in the peripheral direction and are arranged adjoining or near to the outlet opening, wherein they extend away from the edge thereof. Thus, outlet opening can be surrounded by ribs on all sides.

The ribs on the outlet, in particular on the outlet opening, advantageously prevent the escaping baby food concentrate or coffee powder from coming into contact with the housing of the device for preparing baby food concentrate or coffee powder. By the fact that the powder does not touch the housing of the device, the housing does not have to be cleaned after each use and can be used again immediately. In addition, it is avoided that the powder on the housing is contaminated and/or cannot be used to prepare baby food or coffee. The ribs can, however, also serve as a base for the dosing device, in particular if the dosing device is not inserted in the dosing device receiving area of the first receiving area. This enables a simple connection of the first container to the dosing device and a subsequent simple filling of the first container with baby food concentrate or coffee powder.

Preferably, the first receiving area preferably has a rear wall, two spaced apart side walls which are oriented at an angle different from 0° or 180°, in particular essentially transversely to the rear wall, an upper and a limitation which is at an angle of 0° or 180° different angles, in particular are oriented essentially transversely to the side walls, and an open front side opposite the rear wall, so that the first receiving area is formed between the side walls and/or the upper and lower limitation. The first receiving area preferably has a container receiving area for receiving the first container, the container receiving area preferably above the dosing device receiving area is arranged.

The container receiving area can thus adjoin the upper limitation and/or the dosing device receiving area can adjoin the lower limitation. Through the open front, the first container together with the dosing device can thus be introduced into the first receiving area by a essentially perpendicular movement to the rear wall, so that the first container is received by the container receiving area and the dosing device is received by the dosing device receiving area. Preferably, the first container is connected to the dosing device in such a way that the first container, when inserted into the first receiving area, is arranged above or above the dosing device relative to the lower limitation and/or is further spaced from the lower limitation than the dosing device. This enables the powder to be guided from the first container into the dosing device, for example by gravity.

Preferably, a first guide element and a second guide element are arranged between the container receiving area and the dosing device receiving area, the guide elements essentially extending from the open front to the rear wall and/or wherein the guide elements extend away from the side walls.

The guide elements can run essentially continuously from the front to the rear wall. They allow a particularly simple introduction of the first container and the dosing device in the interconnected state into the first receiving area, so that the first container is arranged and/or received above the guide elements and the dosing device is arranged and received below the guide elements. For the correct insertion of the first container and the dosing device, the peripheral wall can be inserted between the guide elements, so that the first and second support surfaces slide essentially along the guide elements. In other words, the first support surface slides along the first guide element and the second support surface slides along the second guide element until the dosing device is completely received by the dosing device receptacle. In the state inserted into the dosing device receiving area of the first receiving area, the lateral contact surfaces of the peripheral wall of the dosing device then rest on the two guide elements. This enables the first container and/or the dosing device to be received in a particularly simple manner and to be arranged in a stable manner in the first receiving area.

Preferably, the guide elements are aligned essentially in a plane parallel to the upper limitation and/or to the lower limitation, the guide elements preferably being inclined towards the front out of the plane towards the container receiving area.

As a result, the guide elements each comprise an insertion bevel, adjoining or near to the open front side, which enables an aid for the correct insertion of the dosing device. In particular, during the introduction two of the ribs which are arranged on the outer wall of the screw conveyor housing can slide essentially along the underside of the guide elements, while the two lateral abutment surfaces slide between the guide elements as previously described. In the state inserted into the dosing device receiving area of the first receiving area, the lateral contact surfaces of the peripheral wall of the dosing device and two of the ribs then rest on the two guide elements. In particular, the contact surfaces can rest against the edges of the guide elements which extend away from the side walls, and the two ribs can rest against the underside of the two guide elements pointing towards the lower limitation.

When the screw conveyor or the screw conveyor housing is inserted into the dosing device receiving area, the auger can click into place, e.g. as soon as the end position has been reached. This means that the user knows that the screw conveyor has been installed correctly or that the (cylindrical) cavity has been correctly connected to the coupling element or the drive shaft. The lead-in bevels can help to bring the first container into the correct position and/or also simplify the click-in of the screw conveyor.

Preferably, the lower limitation has a receptacle for the screw conveyor housing which extends from the open front side to the rear wall.

The receptacle for the screw conveyor housing can extend between the two side walls around a longitudinal axis of the receptacle which is oriented essentially parallel to the two side walls. On the longitudinal axis of the receptacle, particularly adjoining or near the back rear wall, can a receptacle outlet opening be arranged, which is formed with essentially the same shape and the same dimension as the outlet opening of the screw conveyor housing. The receptacle has a cross-section transverse to the longitudinal axis of the receptacle which is essentially concave. In other words, the receptacle is embedded as a essentially concave portion in the lower limitation. The lower limitation can thus have a surface which has a first horizontal surface portion adjoining or near to a first of the side walls and a second horizontal surface portion adjoining or near to the second of the side walls, wherein the receptacle as a essentially concave surface portion between the first and second surface section is arranged.

The receptacle for the screw conveyor housing enables the dosing device to be held particularly securely and firmly in the dosing device receptacle. After inserting and receiving the dosing device, the conveyor screw conveyor housing rests firmly in the receptacle for the conveyor housing, with two of the ribs resting firmly on the first and second horizontal surface sections. At the same time, the outlet opening in the screw conveyor housing is arranged above or adjoining or near to the receiving outlet opening. Thus, the baby food concentrate or coffee powder can be conveyed from the screw conveyor housing through the outlet opening in the screw conveyor housing and the receiving outlet opening in the receptacle in the dosing device receptacle and fed to a preparation device without the baby food concentrate or coffee powder coming into contact with the housing.

Preferably, the drive shaft is formed in or on the rear wall, the drive shaft and the receptacle extending in a plane transverse to the lower limitation.

The coupling element or the drive shaft is preferably arranged in or on the rear wall. The distance between the lower limitation and the coupling element or the drive shaft seen in a plane transverse to the longitudinal direction of the receptacle can correspond to the distance between the first cylindrical cavity and the peripheral wall of the screw conveyor in a plane seen transversely to the longitudinal direction of the screw conveyor. By inserting the dosing device into the receptacle for the screw conveyor housing as previously described, the coupling element or the drive shaft can automatically engage in the (cylindrical) cavity in the conveyor screw conveyor housing. Hence, the screw conveyor can be driven.

Preferably, one or more side walls of the container receiving area comprise a plurality of ribs extending from the one or more side walls.

The plurality of ribs preferably extends essentially parallel to the upper and/or lower limitation. Preferably, the plurality of ribs preferably extend essentially from the open front to the rear wall. However, it is also conceivable that the ribs are arranged transversely to the upper or lower limitation and/or that the ribs do not extend continuously from the front to the rear wall.

The plurality of ribs is preferably arranged in pairs on the two side walls. In other words, two ribs each extend in a plane transverse to the side walls and/or parallel to the upper or lower limitation. In this way, numerous pairs of ribs can be arranged on the side walls in the container receiving area, preferably between the guide elements and the upper limitation. Preferably, the ribs of a pair of ribs are each spaced between approximately 40 and 50 mm from each other, more preferably the ribs of a pair of ribs are each approximately 50 mm apart. It is also conceivable that not all of the ribs of the rib pairs have the same distance from each other, but can have a distance that differs from each other, preferably between approximately 40 and 50 mm.

The ribs enable optimal alignment of the first container received in the first receiving area, so that the baby food concentrate or coffee powder can be guided and/or dosed from an outlet in the first container through the inlet opening of the screw conveyor housing. At the same time, this prevents baby food concentrate or coffee powder from remaining in the first container and not being able to be used for the preparation of baby food or coffee. Thus, the ribs enable a plurality of differently shaped first containers to be easily and securely received, and thereby brought into a certain desired shape so that the powder can shift toward the outlet. The first container is thereby held in a position, in particular in an upright position, in which it does not collapse.

Preferably, a container for receiving and dosing baby food concentrate or coffee powder is provided, the container comprising a housing having an interior space for receiving baby food concentrate or coffee powder, and an outlet in fluid communication with the interior space, which can be connected to an inlet of a dosing device, wherein the dosing device has an outlet, so that by actuating the dosing device, a dosage of the baby food concentrate or the coffee powder is dispensed through the outlet. Preferably, the container is designed to be introduced into a device for preparing baby food or coffee and to be at least partially received by the latter. The dosing device is or can be connected to the container, and the container and/or the dosing device are exchangeable and designed as disposable article.

Preferably, the container for receiving and dosing baby food concentrate or coffee powder is available pre-filled with baby food concentrate or coffee powder. The container can be supplied ex works filled with baby food concentrate or coffee powder, i.e. the container can be filled with baby food concentrate or coffee powder in the factory, so that the container can already be delivered to the consumer filled with baby food concentrate or coffee powder.

The container can be designed to be introduced as the first container into the first receiving area of the device for preparing baby food or coffee as previously described and to be at least partially received therein. Thus, all previously described features of the device, which were described in connection with the first container and/or the dosing device, also apply to the container described below (hereinafter referred to as first container) for receiving and dosing baby food concentrate or coffee powder.

The first container has a dosing device to which the first container can be connected and can thus be connected so that the correct amount of baby food concentrate or coffee powder can be dispensed and thus dosed with the first container and the dosing device. However, it is also conceivable that the first container and the dosing device are two separate elements. Because the first container has an outlet with an outlet opening, baby food concentrate or coffee powder that is received in the first container can exit or be dispensed from the first container. Since the outlet can be connected to the inlet of a dosing device, the baby food concentrate or coffee powder emerging from the first container can be introduced through the inlet of the dosing device. By actuating the dosing device, the baby food concentrate or coffee powder can then exit from a second outlet in the dosing device and be used in the predetermined or predeterminable dosing for preparing baby food or coffee.

Thus, with the described first container, correct dosing of the baby food concentrate or the coffee powder can take place by means of the dosing device. The dosing device can be driven by an actuating and/or drive device. However it is also conceivable that the dosing device is driven manually. Fluid from the second container can be mixed with the baby food concentrate or coffee powder from the first container and introduced into a container, in particular into a baby bottle or into a coffee cup, in the correct mixing ratio. This enables baby food or coffee to be prepared correctly and in a simplified manner.

Furthermore, the first container can have a dosing device to which the first container can be connected and thus connected so that the correct amount of baby food concentrate or coffee powder can be dispensed and dosed using the first container and the dosing device. For this purpose, the first container connected to the dosing device can also be inserted and received in the first receiving area, in particular in the container receiving area and the dosing device receiving area of the device as previously described for preparing baby food or coffee. However, it is also conceivable that the first container and the dosing device are two separate elements, which are each individually inserted into the first receiving area, in particular into the container receiving area and into the dosing device receiving area, and are received separately from each other.

Thus, a correct dosing of the baby food concentrate or the coffee powder from the first container can take place by means of the dosing device. The dosing device can thereby by an actuating and/or drive device, for example in a dosing device receiving area is arranged in the device as previously described, are driven. However it is also conceivable that the dosing device is driven manually. The baby food concentrate or coffee powder can be fed from the first container together with a fluid, for example provided from the second container, to a preparation device and mixed so that the baby food concentrate or coffee powder and the fluid in a correct mixing ratio in a container, in particular in a baby bottle or in a coffee cup. This enables baby food or coffee to be prepared correctly and in a simplified manner.

Preferably, the dosing device comprises a screw conveyor and a screw conveyor housing, the screw conveyor, preferably in its full length, being insertable and rotatable into the screw conveyor housing, so that the screw conveyor and the screw conveyor housing extend around a common screw conveyor longitudinal axis, and the inlet of the dosing device in or is arranged on the screw conveyor housing.

Thus, the configuration of the dosing device enables the baby food concentrate or coffee powder to be guided from the first container into the dosing device and transported by means of the screw conveyor in the screw conveyor housing along the longitudinal axis of the screw conveyor. With each turn of the screw conveyor, a certain amount of powder can be conveyed, so that the dosage of baby food concentrate or coffee powder can be determined by the number of turns. This enables precise and simplified dosing of the baby food concentrate or the coffee powder, which can take place both automatically, for example controlled by a regulating or control device, or manually by an operator.

The first container can be connected to a dosing device which comprises a screw conveyor and a screw conveyor housing. The screw conveyor and the screw conveyor housing can have all the features that were previously described in the context of the device for preparing baby food or coffee, so that the dosing device can be accommodated in the first receiving area or in the dosing device receiving area of the device as previously described.

It is conceivable that the dosing device comprises a plate, which is preferably designed as a standing plate and is arranged on the screw conveyor housing.

This stand plate is used to better position the first container and/or to protect it from falling over, in particular if the first container for holding baby food concentrate or coffee powder is positioned outside the device for preparing baby food or coffee. The plate can be firmly connected to the screw conveyor housing or the plate can be connectable to the screw conveyor housing. Thus, after the baby food concentrate or the coffee powder has been picked up, the plate can be removed from the screw conveyor housing so that the first container and/or the dosing device can be picked up by the first receptacle of the device for preparing baby food or coffee. It is also conceivable that the screw conveyor housing has a casing, the casing having at least one flat surface which serves as a stand plate, so that the first container can be positioned better and is protected from falling over. Preferably, the outlet of the first container is firmly connected to the inlet in the screw conveyor housing, in particular screwed and/or glued.

The first container can be connected to the screw conveyor housing so that baby food concentrate or coffee powder can be introduced from the first container into the screw conveyor housing and/or can be dispensed from it again in the correct dosage. The outlet of the first container can be firmly connected (e.g. glued) to the inlet of the screw conveyor housing. For this purpose, for example, the outlet of the first container can have a peripheral wall which is similar to the peripheral wall of the flange that is arranged on the screw conveyor housing. In particular, the peripheral wall of the container outlet can have a cross-sectional profile which corresponds to the cross-sectional profile of the peripheral wall of the flange, although the circumference of the peripheral wall of the container outlet is slightly larger or slightly smaller than the circumference of the peripheral wall of the flange. In this way, the peripheral walls can be brought into an overlap and/or firmly connected to each other (e.g. glued and/or welded).

However, it is also conceivable that the outlet of the first container with the inlet in conveyor screw conveyor housing is screwed. Thus, the peripheral wall of the flange on the screw conveyor housing can comprise a first drive profile and the peripheral wall of the container outlet can comprise a second drive profile. The first container and the dosing device can preferably be connected to each other in a form-fitting and rotationally fixed manner via the two drive profiles. For example, the outer contour of the peripheral wall of the flange on the screw conveyor housing can have a drive profile and the inner contour of the peripheral wall of the container outlet can have a corresponding drive profile so that the peripheral walls can be connected to each other in a non-rotatable manner in particular. Any structure that enables a connection between the first container and the dosing device can serve as the drive profile. The drive profile can accordingly be polygonal, star-shaped, slot-shaped, etc.

Preferably, the screw conveyor housing is integrated into the first container.

By integrating the screw conveyor housing into the first container, the first container and the screw conveyor housing can be connected integrally to each other so that the first container and the dosing device are in particular firmly and non-detachably connected to each other. It is conceivable that in particular the peripheral wall of the container outlet and the peripheral wall of the flange on the screw conveyor housing are formed integrally with each other.

Preferably, the first container has at least partially a tapering section, the periphery of the first container in the tapering section preferably decreasing essentially conically towards the outlet.

The first container can have a cross-section as viewed in a plane through the longitudinal conveyor screw axis of the conveyor screw conveyor housing in the connected state with the first container, the tapering section being laterally limited by a first side edge and a second side edge becomes. In the “state connected to the first container” means that the dosing device or the screw conveyor housing are connected to the screw conveyor and the first container. The first side edge can run essentially transversely, preferably at an angle of less than 90°, particularly preferably at an angle of approximately 45°, to the longitudinal axis of the conveyor screw conveyor housing (seen in the connected state). The second side edge can run essentially transversely, preferably at an angle smaller than approximately 90°, particularly preferably at an angle of approximately 45°, to the longitudinal conveyor screw axis of the screw conveyor housing. It is also conceivable that both side edges run essentially transversely, preferably at an angle smaller than approximately 90°, particularly preferably at an angle of approximately 45°, to the longitudinal conveyor screw axis of the screw conveyor housing. This arrangement of the side edges relative to the longitudinal axis of the screw conveyor housing (seen in the connected state) enables the baby food concentrate or the coffee powder to be emptied particularly easily from the first container.

Preferably, the second side edge forms an angle of approximately 45° with the first side edge. As a result of this configuration, the circumference of the first container in the tapering section is gradually reduced towards the outlet. This enables a particularly efficient emptying of the baby food concentrate or the coffee powder received in the first container from the outlet and the subsequent introduction into the inlet of the screw conveyor housing.

The first container preferably has at least partially a first essentially symmetrical section, the circumference of the first container remaining the same within the first essentially symmetrical section and wherein preferably the first essentially symmetrical section is spaced further from the outlet than the tapering section.

The first container can have a cross-section in a plane through the longitudinal axis of the screw conveyor housing when connected to the first container, the first essentially symmetrical section being laterally delimited by a first side edge and a second side edge, which are aligned essentially parallel to each other and thus run essentially transversely, preferably at an angle of approximately 90°, to the longitudinal axis of the screw conveyor housing (viewed in the connected state). The first side edge of the first essentially symmetrical section can run in one plane with the first side edge of the tapered section and/or the second side edge of the first essentially symmetrical section can be oriented transversely to the second side edge of the tapered section. However, it is also conceivable that the second side edge of the first essentially symmetrical section runs in the same plane as the second side edge of the tapering section, so that a further tapering section is formed instead of the symmetrical section.

Preferably, the distance between the first and second side edges of the symmetrical section is a maximum of approximately 140 mm and/or the length of the two side edges is a maximum of approximately 155 mm. It is also conceivable that the length of the first side edge is longer than the length of the second side edge. Thus, the length of the first side edge can be a maximum of approximately 155 mm and/or the length of the second side edge can be a maximum of approximately 125 mm.

This embodiment further enables particularly efficient emptying of the baby food concentrate or coffee powder received in the first container from the outlet and subsequent introduction into the inlet of the screw conveyor housing. At the same time, the symmetrical section enables alternative configurations of an inlet for receiving baby food concentrate or coffee powder in the first container.

Preferably, the first container has a second essentially symmetrical section adjoining or near to the outlet, the circumference of the first container remaining the same within the second essentially symmetrical section and essentially corresponding to the periphery of the outlet and/or an outlet opening in the outlet.

The first container can have a cross-section in a plane through the longitudinal conveyor screw axis of the screw conveyor housing when connected to the first container, the second essentially symmetrical section being laterally delimited by a first side edge and a second side edge which are aligned essentially parallel to each other and thus run essentially transversely, preferably at an angle of approximately 90°, to the longitudinal conveyor screw axis of the screw conveyor housing (viewed in the connected state). The first side edge of the second essentially symmetrical section can run in one plane with the first side edge of the tapered section and with the first side edge of the first essentially symmetrical section and/or the second side edge of the second essentially symmetrical section can be transverse to the second Side edge of the tapered portion be aligned and parallel to the second side edge of the first essentially symmetrical portion.

Preferably, the distance between the first and second side edges of the second symmetrical section is in the range from approximately 20 mm to 60 mm (e.g. approximately 50 mm) and/or the length of the two side edges is in each case in the range from approximately 10 mm to 110 mm (e.g. approximately 15 mm or 90 mm each).

Preferably, the second essentially symmetrical section is connected to the outlet, so that the diameter of the outlet or the passage of the outlet opening preferably corresponds to the distance between the first and second side edges of the second symmetrical section.

This embodiment further enables particularly efficient emptying of the baby food concentrate or coffee powder received in the first container from the outlet and subsequent introduction into the inlet of the screw conveyor housing.

However, it is also conceivable that the first container has a further, essentially symmetrical section instead of the tapering section. In this case, the first side edges of the three sections can run in one plane and the second side edges can run in one plane, the two planes being oriented essentially parallel to each other.

Preferably, the first container has an inlet opening, the inlet opening preferably being arranged essentially opposite the outlet and/or an outlet opening in the outlet.

Preferably, the inlet opening can be arranged in the first essentially symmetrical conveyor screw section. More preferably, the inlet opening can be arranged adjoined to or near to a side edge which runs between the first and second side edges of the first essentially symmetrical section. The inlet opening is preferably arranged at a first free end of the first container which lies opposite a second free end of the first container, the outlet and the outlet opening being arranged at the second free end. The tapering section can be arranged between the inlet or the inlet opening and the outlet or the outlet opening.

Baby food concentrate or coffee powder can be received in the first container through the inlet opening. By arranging the inlet opening opposite the outlet, the baby food concentrate or coffee powder can be guided in the direction of the outlet and the outlet opening and can be fed out of the first container into the dosing device. This enables the correct dosage of the baby food concentrate or the coffee powder. The inlet opening can preferably be closed by means of a closure element, more preferably by means of a zipper or zipper.

However, it is also conceivable that the first container does not have an inlet opening and is integrally or firmly connected to the dosing device. The first container and the dosing device can be connected to each other in one piece as a unit and filled with baby food concentrate or coffee powder.

Preferably, the inlet opening extends adjoining or near to the first free end between the first and second side edges of the first essentially symmetrical section. Preferably, the inlet opening can be closed with a closure element. Thus, the first container is advantageously reusable and/or baby food can be refilled after it has been completely emptied, or the first container can be closed again after transferring baby food concentrate or coffee powder. However, it is also conceivable that the first container is not reusable and does not have a closure element, since the inlet or the inlet opening is welded after the baby food concentrate or the coffee powder has been taken up. It is also conceivable that the first container does not have an inlet or an inlet opening, but that the baby food concentrate or the coffee powder is initially received into the first container through the outlet or the outlet opening, and the outlet is then connected to the dosing device. In particular, after the baby food concentrate or the coffee powder has been taken up, the outlet can be connected to the inlet of the dosing device by means of a connecting element, for example an adhesive element in the form of an adhesive strip or a clip. In this case, one and the same opening is used to receive the baby food concentrate or the coffee powder in the first container and to remove the baby food concentrate or the coffee powder from the first container.

Thus, the first container with baby food concentrate can already be supplied connected to the dosing device and is designed as a disposable or single-use articles. It is also conceivable that the dosing device, which can be connected to the first container, is designed as a reusable article. In particular if the dosing device and the first container are formed integrally with each other or are glued or screwed to each other, the dosing device can be designed as a single-use or disposable articles.

The closure element can be designed as a zipper that is easy to open and close. However, it is also conceivable that instead of the zipper or in addition to the zipper, a rail is arranged on the first free end of the first container. With this rail, the first container can be connected to an upper area of the first receiving area. One or more magnetic holders, one or more Velcro fasteners, one or more buttons and/or one or more adhesive strips or other types of fastenings with which the first container can be connected to the upper area of the receiving area are also conceivable. It is also conceivable that the first container has a first screw element and the upper area of the first receiving area has a second screw element, so that the first container can be connected to the upper area of the receiving area by means of the screw elements.

A tab can be arranged adjoined or near to the closure element. The tab may have an internal opening. The inner opening can be designed as a handle, so that the first container can be carried or held in a simplified manner from one location to another. The inner opening can, however, also serve to be hooked or suspended in a hook, for example, which ensures additional stability, in particular when filling the first container. Preferably, the closure element, preferably the zipper, is preferably designed to be inserted into a groove in a first receiving area of a device for preparing baby food or coffee.

The closure element or the zipper can be designed to be inserted (at least partially) into a groove. The closure element or the zipper is preferably designed to be inserted into a groove which is arranged in the first receiving area, in particular on the inside of the upper limitation which points towards the lower limitation. The groove can be in essentially the same plane as the drive shaft in the dosing device receiving area and how the feed screw longitudinal axis of the feed screw conveyor housing run when inserted into the device. The groove preferably extends at least partially in the upper limitation. More preferably, the groove extends from an area adjoining or near to the open front side to an area adjoining or near to the rear wall. This enables the first container and/or the dosing device to be easily inserted into the first receiving area of the device for the preparation of baby food, the dosing device being received by the receiving device in the lower limitation and the cylindrical cavity of the dosing device being able to engage with the drive shaft in the drive device. At the same time, the closure element or the zipper can be inserted into the groove, which enables an additional hold for the first container in addition to the lateral ribs.

The first container can be made from different materials and can comprise, for example, paper, plastic or other flexible materials for holding powdery materials such as baby food concentrate or coffee powder. Furthermore, the first container can be configured as a pouch or a bag. However, it is also conceivable that the first container is made of a non-flexible material and is therefore dimensionally stable, it being possible for it to comprise a metal such as aluminum or a plastic, for example. For example, the first container can also be designed as a cardboard box, for example a Tetra Pak. In particular, when the first container is made of a non-flexible material, the second receiving area can have two open sides, which are arranged on opposite sides of the longitudinal axis of the receiving area, instead of the closed side walls and the plurality of ribs which extend away from the side walls.

The first container can have a capacity of approximately 1.5 dm3. This capacity allows up to 500 g baby food concentrate or coffee powder to be taken, with 500 g baby food concentrate or coffee powder corresponding to a volume of 1.1 dm3. Thus, the capacity of 1.5 dm3 allows convenient filling and/or transferring of baby food concentrate or coffee powder. However, it is also conceivable that the first container has a capacity that deviates from approximately 1.5 dm3, so that the first container can be made larger or smaller.

The second receiving area and the second container are described in more detail below:

Preferably, the second receiving area preferably has a rear wall, two side walls which are spaced apart from each other and which are oriented at an angle different from 0° or 180°, in particular essentially transversely to the rear wall, a lower limitation which is at an angle different from 0° or 180° is aligned in particular transversely to the side walls, and an open top opposite the lower limitation, the second receiving area for receiving the second container being formed between the side walls.

The second receiving area can comprise an open top. In other words, the top can be designed to be completely open. This enables the second container to be introduced into the second receiving area by an essentially perpendicular movement to the lower limitation, so that the second container can be received by the second receiving area. However, it is also conceivable that the second receiving area has an upper limitation in which a through-hole or opening is arranged, through which the second container can be introduced into the second receiving area by a movement essentially perpendicular to the lower limitation.

The second receiving area can have a front side opposite to the rear wall, which can preferably comprise a window element, for example a window element made of glass or plastic, or a flap or closure flap. This enables the filling level of the second container to be checked easily through the front. However, it is also conceivable that the front side is designed as a front wall which, like the rear wall, is closed and has no opening. The open front of the first receiving area can also be closed by means of a flap or cap, preferably in a manner analogous to the closing flap of the second receiving area, be closed. Thus, after the first container has been inserted and picked up, the first receiving area can be protected from dust or dirt by closing the closure flap.

Preferably, the at least one, second receiving area is preferably designed to receive a lifting system for dosing fluid.

By means of the lifting system, a pressure can be exerted on the second container for a fluid, for example with the aid of a pump mechanism, so that the fluid can be dosed correctly. However, it is also conceivable that a pressure can be exerted on the second container with the aid of a rotary mechanism or some other mechanism, so that the fluid can be dosed correctly and in a particularly easy-to-implement manner. The second receiving area is designed in such a way that it can also accommodate the lifting system in addition to the second container.

Preferably, the lifting system is preferably connected or can be connected to the second container.

The lifting system can be firmly connected to the second container. In other words, the lifting system can be integrated into the second container and offered or delivered in such an integrated manner. However, it is also conceivable that the lifting system and the second container are two separate elements that can be combined or connected to each other so that the fluid can be dosed from the second container. For example, the lifting system can be connected or connectable to an opening of the second container, for example to the inlet or the outlet of the second container. Like the second container, the lifting system can be interchangeable and a disposable or single-use articles. The lifting system can thus be delivered from the factory together with the second container, which is preferably already filled with fluid.

Preferably, the lifting system is connected to the outlet of the second container or connectable. Thus, by actuating the lifting system, the fluid can be pumped from the second container and precisely dosed. The lifting system is preferably connected or connectable to a container or cup, for example for coffee or baby food, or a preparation device, for example via a hose system. In this way, the correctly dosed fluid can be filled into the container or into the cup or into the preparation device.

Preferably, the container or cup or the preparation device is arranged in the direction of gravity below the lifting system that can be or is connected to the second container. The lifting system is preferably arranged or positioned in the direction of gravity between the container or cup or preparation device and the second container. Thus, the fluid can be guided in a simple manner from the second container in the direction of the lifting system by gravity and pumped out of the second container into the container or cup or to the preparation device, so that the fluid is particularly simple in the container or cup or can be guided into the preparation device.

Preferably, the lifting system has a piston and a rotary plate.

It is conceivable that the lifting system can dose the fluid by means of a piston pump. For example, the lifting system can comprise a rotary plate which is driven by means of a motor and can exert a pressure on a piston. The piston can be connectable or connected to the second container in such a way that the piston can be deflected or moved by the rotary plate. Preferably, the rotary plate is arranged above the piston in the direction of gravity, so that the rotary plate can be driven by the motor and set in a rotary motion. This rotary movement sets the piston in a translatory movement. Thus, the piston can be pressed downward in the direction of gravity in the direction of the second container so that the fluid can be pumped out of the second container and correctly dosed. Thus, the fluid can easily be removed from the piston by the deflection or movement of the piston second container can be pumped and correctly dosed. One revolution of the motor can lead to several lifting movements. In other words, one revolution of the motor can lead to several combined rotary and translational movements of the rotary plate and the piston.

Preferably, the turntable is designed as an eccentric or as a control disk which is attached to a shaft and whose center point lies outside the shaft axis. Preferably, the piston is arranged below the eccentric in the direction of gravity and outside, preferably above or below in the direction of gravity, its shaft axis. In this way, the rotational movement of the eccentric can advantageously be converted into the translational movement of the piston or into the piston stroke.

Preferably, the second receiving area has a lifting system receiving area.

The lifting system receiving area is designed to receive the motor and/or the rotary plate and/or the piston. It is conceivable that the motor and/or the rotary plate and/or the piston are firmly connected to the lifting system receiving area. These can be arranged, for example, on the rear wall of the second receiving area and/or on its side walls. The second container can then be introduced and inserted into the second receiving area in such a way that the rotary plate and/or the piston in the lifting system receiving area can interact or interact with the second container. Thus, by means of the rotary plate and the piston, the fluid can be pumped from the second container and dosed correctly. It is also conceivable that the motor and/or the rotary plate and/or the piston are firmly connected to the second container and can be exchanged together with the second container. Thus, the motor and/or the rotary plate and/or the piston can be inserted together with the second container into the lifting system receiving area, so that the fluid can be pumped and dosed out of the second container.

Preferably, the lifting system has a sensor.

The sensor can be firmly connected to the lifting system receiving area. The fill level of the fluid in the second container can be determined by means of the sensor when the second container is introduced or inserted into the second receiving area. The sensor can be connected to application software, for example a mobile app, as described later, so that new fluid can be ordered automatically on the Internet based on the fill level of the fluid.

Preferably, the second container is connected or connectable to a dosing device or the second container comprises a dosing device, the dosing device preferably being a lifting system, the lifting system being designed to dose the fluid from the interior of the second container.

It is conceivable that the device for dosing and/or preparing baby food, in particular baby milk or baby food, or coffee, comprises only one dosing device for dosing the fluid and no dosing device for dosing the baby food concentrate or the coffee powder. In the case of the device for dosing and/or preparing baby food, the fluid could then be a preprepared baby milk and thus not just water.

Preferably, the lifting system is made of a bioplastic or bioplastic or a bio-based plastic. The lifting system preferably comprises a bioplastic or bioplastic or a bio-based plastic. For example, the bioplastic can comprise stone paper and/or wood.

Preferably, at least one of the side walls of the second receiving area is preferably an inclined side wall which is inclined at an angle different from 90°, preferably at an angle between 10° and 50°, more preferably at an angle between 10° and 30°, particularly preferably at an angle of 20° to the lower limitation.

The inclined side wall can have all the advantages and features of the device for dosing and grinding coffee beans and/or for preparing coffee in the embodiment as previously described. Furthermore, the device for dosing and/or preparing baby food or coffee can be designed as in the previously described embodiment of a device for dosing and grinding coffee beans and/or for preparing coffee, so that it can have an inclined side wall or with this can work together.

Preferably, the inclined side wall can preferably be connected or connectable to the rear wall and can be spaced from the lower limitation. Thus, the lower edge of the inclined side wall or the edge of the inclined side wall, which points to the lower limitation of the second receiving area, can be arranged adjoining or near to a flange which can surround a through-hole in the lower limitation. The inclined side wall can be configured to receive the second container of fluid and to hold it in the inclined position. In other words, one of its outer side walls of the second container can rest against the inclined side wall, so that the inclined side wall is designed as a support element and/or as a support element for the second container. The second container can be held in an inclined position by the inclined side wall, so that the outlet of the second container can open into the through-hole, which can be surrounded by a flange. The flange enables a simplified introduction of the outlet of the container into the through-hole and at the same time serves as a lateral support or as a lateral support element for the outlet. The inclined position of the second container is advantageous since the fluid can thus flow out of the second container in a suitable manner, so that no residual volume or residual fluid or dead volume remains in the second container. At the same time, the first container with the dosing device can thus be arranged in a position which is arranged essentially transversely, preferably at an angle of 90° to the lower limitation and above the lower limitation and/or above the through hole.

Thus, the outlet of the screw conveyor housing and the outlet of the second container can advantageously open together in the through-hole. This enables the coffee powder or the baby food concentrate with the fluid to be guided through the through-hole in the device for dosing and/or preparing baby food or coffee, so that the coffee powder or the baby food concentrate and the fluid are in a container, preferably a baby bottle or a filter container, can be fed.

Preferably, a plurality of clamping elements is arranged adjoining or near to the side walls, which clamp elements extend at least partially between the front side opposite the rear wall and the rear wall of the second receiving area. The clamping elements are preferably designed as clamps. Two of the clips can be arranged opposite each other and in a plane parallel to the lower limitation of the second receiving area.

Particularly preferably, at least two clamping elements, preferably three clamping elements, are arranged adjoining or near to one of the two side walls of the second receiving area and at least two clamping elements, preferably three clamping elements, are arranged adjoining or near to the other of the two side walls of the second receiving area. In other words, the second receiving area preferably comprises up to six clamping elements, two of the six clamping elements being arranged opposite to each other as a pair of clamping elements in a plane parallel to the lower limitation of the second receiving area. The second receiving area preferably comprises up to three pairs of clamping elements. However, it is also conceivable that the second receiving area has more than three clamping elements adjoining or near to the two side walls, so that the second receiving area has more than six clamping elements and thus more than three pairs of clamping elements.

The clamping elements or the clamping element pairs enable the fluid to be dosed in the interior of the second container or from the interior of the second container.

Due to the desired or correct dosage of the fluid in the interior of the second container can be clamped off by the clamping elements or the clamping element pairs. This is advantageous because expensive peristaltic pumps, flow sensors, etc. can be dispensed with.

It is conceivable that the fluid from the interior of the second container can be dosed as a function of time. This enables alternating dosing of baby food concentrate or baby food powder from the first container and of fluid from the second container is thus made possible. This serves to improve the mixing behavior and/or the mixing ratio between the baby food concentrate or baby food powder and the fluid. A particularly good mixing ratio is obtained when first the fluid, then the powder, and then the fluid is dosed again. Mixing is preferably carried out in this order at a temperature of approximately 37 degree. C. to 43 degree. C., particularly preferably approximately 40 degree. The mixture of powder and fluid can then be shaken manually or automatically. The baby food concentrate can preferably also be dosed from the first container as a function of time.

At this point it should be noted that the baby food concentrate or the coffee powder can be dosed in an analogous manner inside the first container by means of a plurality of clamping elements or by means of clamping element pairs, as described below in connection with the second container and the dosing of the fluid. In other words, the baby food concentrate or the coffee powder cannot be dosed inside the first container by means of the screw conveyor and the screw conveyor housing; with which the baby food concentrate or coffee powder can be correctly dosed.

By introducing the second container through the open top into the second receiving area, the second container is received by the second receiving area in such a way that the second container can be held and/or clamped laterally by the up to three pairs of clamping elements. The clamping element pairs clamp the fluid inside the second container. Due to the fact that the clamping elements are arranged displaceably on the side walls of the second receiving area, the fluid can be dosed by moving the clamping elements or pairs of clamping elements. Thereby, the up to three pairs of clamping elements can assume at least one position, in particular a first position and a second position. In the first position, up to three pairs of clamping elements can laterally adjoin the second container and/or touch the second container in such a way that the clamping element pairs can exert pressure on the side walls of the second container, in particular on two opposite side walls of the second container. In the second position, the up to three pairs of clamping elements cannot adjoin the container or touch the second container, so that the pairs of clamping elements cannot exert any pressure on the side walls of the second container. The different positions of the at least one pair of clamping elements are advantageous if the fluid in the interior of the second container is to be tempered or heated or sterilized by means of the tempering device. Due to the heating by the boil-off, the fluid in the interior of the second container expands, so that the periphery of the outer wall and thus the distance between the side walls of the second container increases due to the expanding fluid.

Due to the first position and the second position of the at least one pair of clamping elements, the position of the clamping elements can be changed or displaced relative to the side walls of the second receiving area. Thus, the position of the clamping elements can also be changed relative to the side walls of the second container when the second container is received in the second receiving area. Due to the arrangement of the clamping element pairs, the second container and/or the fluid in the interior of the second container cannot come into contact with the side walls of the second receiving area when the second container is received in the second receiving area. This reduces soiling of the side walls of the second receiving area due to the introduction of the second container as well as contamination of the fluid inside the second container.

A first of the pairs of clamping elements can be arranged in such a way that the clamping elements of the first pair of clamping elements have a first distance from the lower limitation. A second of the clamping element pairs can be arranged in such a way that the clamping elements of the second clamping element pair have a second distance from the lower limitation which is greater than the first distance from the lower limitation. The first pair of clamping elements can thus be arranged as a lower clamping element pair adjoining or near to the lower limitation in the second receiving area. The second pair of clamping elements can be arranged as an upper clamping element pair adjoining or near to the open upper side. Furthermore, a third of the clamping element pairs can be arranged as a middle clamping element pair between the first clamping element pair and the second clamping element pair and can have a third distance to the lower limitation that is greater than the first distance and less than the second distance.

The first, lower pair of clamping elements can have a first distance from the lower limitation which is between 10 mm and 30 mm, preferably approximately 20 mm. The second, upper pair of clamping elements can have a second distance to the lower limitation which is between 160 mm and 240 mm, preferably approximately 180 mm. The distance between the first, lower pair of clamping elements and the second, upper clamping element pair can preferably be between 140 mm and 220 mm, preferably approximately 160 mm. The third, middle pair of clamping elements can be arranged between the first, lower pair of clamping elements and the second, upper clamping element pair, so that it is in a range between 10 mm and 30 mm, preferably approximately 20 mm, and a range between 160 mm and 240 mm, preferably approximately 180 mm from the lower limitation.

The first, lower pair of clamping elements can exert pressure on the side walls of the second container when the second container is received in the second receiving area or clamp or clamp the side walls of the second container in such a way that the second container is closed and no fluid from the second container, for example through an outlet opening in the second container, can emerge when the second container is received in the second receiving area. The first, lower pair of clamping elements can thus seal the second container in a sterile manner, so that no bacteria or germs can enter the second container, for example through the outlet opening in the second container. The second, upper pair of clamping elements can exert pressure on the side walls of the second container or clamp or clamp the side walls in such a way that the second container is closed and no fluid from the second container and/or from a fluid reservoir that can be connected to the second container, for example through an inlet opening in the second container, when the second container is received in the second receiving area. The second, upper pair of clamping elements can thus seal the second container in a sterile manner, so that no bacteria or germs can enter the second container, for example through an inlet opening in the second container.

The first, lower pair of clamping elements and the second, upper pair of clamping elements close off the area to be sterilized or heated in the interior of the second container and thus store the fluid in a sterile manner. In this way, the preparation time can be reduced as the fluid does not have to be boiled to kill any germs. The fluid only needs to be brought to drinking temperature. The third, middle pair of clamping elements is provided for dosing the fluid in the interior of the second container between the first, lower clamping element pair and the second, upper clamping element pair.

Preferably, the first, lower pair of clamping elements and the second, upper clamping element pair are preferably arranged in such a way that the two clamping element pairs delimit an area of the second container which limits an amount of fluid of approximately 180 ml to 250 ml in the second container. This makes it possible to dose an amount of fluid up to approximately 250 ml. This represents the maximum amount of fluid that can be used to prepare baby food or coffee and that can be taken up by a conventionally available baby bottle or coffee cup. However, it would also be conceivable that the first, lower clamping element pair and the second, upper clamping element pair are arranged in such a way that the two clamping element pairs delimit an area of the second container which limits an amount of fluid of more than 250 ml, preferably more than 250 ml and up to approximately 500 ml or more than 500 ml and up to 750 ml, in the second container. In this way, an amount of fluid could also be provided or dosed which is suitable for preparing baby food for more than one conventionally available baby bottle or for preparing coffee for more than one portion or coffee cup. Thus, several baby bottles could be filled with baby food in a short time, or several coffee cups or coffee pots could be filled with coffee in a short time.

If at least one of the side walls of the second receiving area is designed as an inclined side wall, the clamping elements can preferably be designed as brackets, the brackets being arranged in a plane parallel to the inclined side wall. The clamps can be arranged in the plane parallel to the inclined side wall, as described in the previously described embodiment for the device for dosing and grinding coffee beans and/or for preparing coffee. Furthermore, the clips can have all of the advantages and features described in this context.

Preferably, one of the clamping elements is preferably replaced by the tempering device for controlling the temperature of the fluid to be dosed by means of the clamping elements. This enables simultaneous dosing and tempering of the fluid in the container by at least one of the clamping elements.

Preferably, the distance between the clamping elements can be changed relative to the lower limitation and/or relative to the open upper side.

Preferably, the third, middle pair of clamping elements is adjustable in height. In other words, the third distance to the lower limitation can be changed. This enables the exact dosage of the desired amount of fluid for the preparation of baby food. It is further conceivable that the first, lower pair of clamping elements and the second, upper clamping element pair are height-adjustable, so that the first and second distance to the lower limitation can be changed. This enables the two pairs of clamping elements to be adapted to the size or volume of the second container so that different sizes of the second container can be received by the second receiving area and held or limited laterally by the first, lower clamping element pair and by the second, upper clamping element pair can, so that the fluid can be correctly dosed inside the second container.

Preferably, each of the clamping elements comprises a first clamping element area and a second clamping element area, the clamping element areas being arranged on opposite sides of a clamping element longitudinal axis.

The first clamping element surface and the second clamping element surface can be arranged essentially parallel to each other and can each extend between a first end and a second end. The first clamping element surface can extend in a first plane and the second clamping element surface can extend in a second plane, wherein the first plane and the second plane are aligned parallel to each other and/or wherein the clamping element longitudinal axis is in a plane between the first plane and the second level is arranged. The width of the two clamping element surfaces, i.e. the width of the two clamping element surfaces at an angle different from 0° or 180°, in particular essentially transversely to the longitudinal axis of the clamping element, tapers from the first end to the second end. Furthermore, each of the clamping elements can comprise a connecting plate which is arranged at an angle different from 0° or 180°, in particular essentially transversely to the longitudinal axis of the clamping element. The first clamping element surface can be connectable to the connecting plate by means of the first end, and the second clamping element surface can be connectable to the connecting plate by means of the first end. The connecting plate is designed to connect the individual clamping elements to the second receiving area.

In particular, the connecting plate can be connectable to the rear wall of the second receiving area so that the clamping elements extend essentially transversely to the rear wall at an angle different from 0° or 180°, in particular essentially transversely, so that the second ends of the clamping element surfaces are spaced from the rear wall. Preferably, the connecting plate of each of the individual clamping elements is connected to the rear wall adjoining or near to one of the side walls of the second receiving area, so that the clamping elements extend along the side walls between the front side and the rear wall. This enables the second container to be held between the individual clamping elements of the clamping element pairs after it has been received in the second receiving area and the fluid can be dosed inside the second container. Due to having the width of the two clamping element surfaces tapers towards the second end, the individual clamping elements can be brought from the first position to the second position in a particularly simple manner. However, it is also conceivable that the individual clamping elements are not connected to the rear wall by means of a connection plate, but that the clamping elements are arranged or displaceably arranged on the rear wall and/or on the side walls of the second receiving area by means of a carriage or by means of a rail or guide rail element are connectable.

Preferably, the two clamping element surfaces are connected by means of a third clamping element surface, the third clamping element surface having an essentially conically shaped cross section essentially transversely to the longitudinal axis of the clamping element.

The third clamping element surface can extend from a first side edge of the first clamping element surface to a first side edge of the second clamping element surface. The first side edges of the first and second clamping element surfaces can extend in the same plane, which extends at an angle different from 0° or 180°, preferably at an angle of 90°, in particular transversely to the longitudinal axis of the clamping element. The third clamping element surface can be at an angle of 90° to the first clamping element surface and be arranged to the second clamping element surface and/or at an angle of 90° to the connecting plate and/or at an angle of 90° to the rear wall of the second receiving area when the connecting plate is connected to the rear wall. Each of the clamping elements of the individual clamping element pairs can thus have a third clamping element surface, the third clamping element surfaces of the two clamping elements of each clamping element pair being aligned parallel to each other when the clamping elements are connected to the rear wall by means of the connecting plate.

Preferably, the connecting plate comprises at least one through hole so that the clamping elements can be connected to the rear wall by means of a connecting element, for example a screw. However, it is also conceivable that the connecting plate is arranged adjoining or near to the first end of the clamping element surfaces, for example on second side edges opposite the first side edges of the clamping element surfaces, so that the clamping elements can be connected to the side walls.

Preferably, the third clamping element surface can have a essentially conically shaped or triangular shaped cross section at an angle different from 0° or 180°, in particular essentially transversely to the longitudinal axis of the clamping element. The third clamping element surface can have a clamping element edge which extends essentially in the direction of the longitudinal axis of the clamping element and due to the essentially conically shaped cross section between the first side edge of the first clamping element surface and the first side edge of the second clamping element surface. Preferably, the clamping element edge extends in the same plane as the clamping element longitudinal axis. Due to the design of the clamping element edge of the individual clamping elements, the fluid in the second container can be dosed particularly well when the second container is received in the second receiving area and pressure is exerted on the side walls of the second container by means of the clamping elements or the clamping element edge.

It is also conceivable that the third clamping element surface has more than one clamping element edge, preferably two clamping element edges that are like the previously described clamping element edge extend essentially in the direction of the clamping element longitudinal axis and extend between the first side edge of the first clamping element surface and the first side edge of the second clamping element surface. The clamping element edges each extend in a plane which runs essentially transversely or at an angle different from 0° or 180°, preferably at an angle of 90°, to the plane of the clamping element longitudinal axis.

Each of the clamping elements can be designed to be open opposite to the third clamping element surface and viewed transversely to the clamping element longitudinal axis. In other words, each of the clamping elements comprises an inner cavity which is delimited by the three clamping element surfaces and has an open side. When the clamping element is connected to the second receiving area, for example when the clamping element is connected to the rear wall by means of the connecting plate, the open side of the inner cavity points towards one of the two side walls of the second receiving area. As a result of this configuration, the clamping elements have a reduced weight and are also suitable for clamping the second container and/or for dosing the fluid inside the second container. However, it is also conceivable that the clamping element has a fourth clamping element surface opposite the third clamping element surface, seen transversely to the clamping element longitudinal axis, which extends between a second side edge of the first clamping element surface and a second side edge of the second clamping element surface.

Preferably, at least one of the clamping element surfaces, preferably the third clamping element surface, is preferably designed as a support surface, preferably as a rubberized support surface.

The support surfaces enable the second container to be closed particularly tightly. The support surfaces can be designed as rubberized bearing surfaces and comprise an elastomer or a thermoplastic or a thermosetting plastic or be made of such a material. The support surface can be a comprise soft plastic or a solid plastic or be formed from a soft plastic or from a solid plastic. in particular, when the first, lower pair of clamping elements and the second, upper pair of clamping elements have assumed the second position, the rubberized contact surface enables an improved seal, so that no germs or bacteria can penetrate into the interior of the second container and the fluid is received in a sterile manner inside the second container is. Furthermore, it is ensured that the interior of the device, in particular the interior of the second receiving area, for example the side walls, do not come into contact with the fluid. It is thus possible to dispense with cleaning the device, in particular cleaning the interior of the device.

Preferably, one or more (preferably each of the) clamping elements preferably has at least one spring element.

The at least one spring element can be designed as a tension spring or as a rubber band, which is arranged adjoining or near to the first end of the first clamping element surface or adjoining or near to the first end of the second clamping element surface. However, it is also conceivable that a first spring element is arranged adjoining or near to the first end of the first clamping element surface and a second spring element is arranged adjoining or near to the first end of the second clamping element surface. The contact pressure of the clamping elements in the first state can be adjusted by the spring element. This enables a particularly tight closure or sealing of the second container, in particular by the first, lower clamping element pair and by the second, upper clamping element pair. The edge of the clamping element can in each case be pressed particularly tightly against the side walls of the second container so that the fluid is received in a sterile manner in the second container. In particular, each of the clamping elements of the individual clamping element pairs can comprise a spring element. This double-sided spring support on both sides of the second container makes a good and sterile one sealing guaranteed. The sterility is important because the water is stored or kept in the second container after boiling or after tempering to a temperature of at least 100° C. or to the boiling temperature. When the baby food is being prepared, the water is then only brought to the intended drinking temperature, i.e. heated or cooled, so that the user can save time during preparation.

A spring support on both sides enables a flat pressure or a uniform surface pressure, which enables a particularly sterile seal. The flat pressure or uniform surface pressure can exert a pressure on the contact surfaces of the clamping elements which can be greater than the hydrostatic pressure of the fluid inside the second container or greater than the pressure resulting from the heating or boiling of the fluid. In this way, tightness or sealing of the second container is ensured at all times by means of the clamping elements and the spring support provided thereon.

Preferably, the tempering device is arranged in contact with the second container, the tempering device preferably being arranged in an area adjoining or near to the lower limitation of the second receiving area and/or with the tempering device in an area adjoining or near to one of the clamping elements, the closest to the lower limitation is arranged.

The tempering device can be arranged adjoined or near to the first, lower pair of clamping elements. When the second container is received by the second receiving area, a lower area of the second container is arranged adjoining, preferably adjacent, to the tempering device. Preferably, the tempering device can comprise a tempering element, for example a heating plate, which is arranged adjoining or near to the lower limitation of the second receiving area and/or adjoining or near to a clamping element of the first, lower clamping element pair. It is also conceivable that the tempering element between the first, lower clamping element pair and the third, middle clamping element pair is arranged, or that the tempering element is arranged between the first, lower clamping element pair and the second, upper clamping element pair. The tempering element can extend between a clamping element of the first, lower clamping element pair and a clamping element of the third, middle clamping element pair or a clamping element of the second, upper clamping element pair. It is also conceivable that the device comprises more than one tempering element, preferably two tempering elements, which are each arranged near to the opposite side walls of the second receiving area and in a position to the lower limitation and/or the second container as previously described. For example, a first of the tempering elements can be arranged adjoining or near to a clamping element of the first, lower clamping element pair and a second of the tempering elements can be arranged adjoining or near to the other clamping element of the first, lower clamping element pair.

Due to this arrangement of the at least one tempering element allows the fluid to be mixed in the interior of the second container. The deepest or lowest point of the second container or the point of the second container which is closest to the lower limitation of the second receiving area when the second container is accommodated in the second receiving area is tempered or heated. A circulating movement of the fluid in the interior of the second container can thus be set in motion and the fluid is thoroughly mixed in the interior of the second container. This ensures that the fluid can be kept at the same temperature throughout the interior of the second container. This is advantageous because a mixing unit in the second container can be dispensed with.

Preferably, the tempering device comprises at least one sealing element, preferably two sealing elements. The at least one sealing element can be a sealing lip, which is arranged adjoining or near to the lower limitation of the second receiving area and/or is arranged adjoining or near to one of the clamping elements of the first, lower clamping element pair and/or is arranged adjoining to or near to one of the tempering elements. The sealing lip is designed to press the deepest, lowest point of the second container in the state received in the second receiving area, preferably the area adjoining or near to an outlet opening of the second container, against the tempering element, so that the tempering element is in contact, preferably in a surface-to-surface contact is brought to the second container. However, it is also conceivable that, instead of the separate sealing element, one of the clamping elements of the first, lower pair of clamping elements, the deepest, lowest point of the second container in the state received in the second receiving area, preferably the area adjoining or near to an outlet opening of the second container to which can press the tempering element. This creates a particularly high thermal conductivity and the temperature of the fluid inside the second container is particularly effective. In the state of the second container received in the second receiving area, the sealing element and a clamping element of the first, lower clamping element pair can be arranged on opposite sides of the second container.

The at least one tempering element can be designed as a heating element as previously described, for example as a heating plate, in order to set a circulating movement of the fluid in motion inside the second container and to heat the inside evenly. Preferably, the at least one tempering element is designed to heat the fluid to a temperature of at least 100° C. or to the boiling temperature. In the case of dosing and/or preparing baby food, this enables the fluid inside the second container to be sterilized so that germs and bacteria are reliably killed and the fluid is suitable for preparing baby food or coffee. In the case of the dosing and/or preparation of coffee, the at least one tempering element also enables the fluid to be heated to a temperature between 90° C. and 100° C., particularly preferably approximately 96° C., suitable for the preparation of coffee.

However, it is also conceivable that the at least one tempering element is designed as a cooling element, for example as a cooling plate. Hence, the circulating movement in the interior can be stopped by the cooling element and the fluid can be cooled to a specified temperature. It is also conceivable that a first of the tempering elements is designed as a heating element, for example as a heating plate, and a second of the tempering elements is designed as a cooling element, for example as a cooling plate. Furthermore, one and the same tempering element can be designed both as a heating element and as a cooling element. For example, when dosing and/or preparing baby food after the fluid has been sterilized by heating the fluid to at least 100° C. or to the boiling temperature, the fluid can be brought to the intended temperature for the preparation of baby food or coffee or to the intended drinking temperature. During the heating to at least 100° C. or to the boiling point, the third, middle pair of clamping elements can be brought into the second position, and during the subsequent cooling, for example by the tempering element, to the intended drinking temperature, the third, middle pair of clamping elements can be brought into the first Position. Thus, the second container is held and/or clamped by the clamping elements both during heating and during cooling. Correct dosing of the correctly tempered fluid is thus guaranteed.

If at least one of the side walls of the second receiving area is designed as an inclined side wall, the tempering device can also preferably be arranged in an area adjoining or near to the inclined side wall and/or adjoining or near to one of the clamping elements that is closest to the lower limitation.

In this case, the tempering device can be arranged on the inclined side wall in such a way as described in the previously described embodiment for the device for dosing and grinding coffee beans and/or for preparing coffee. Furthermore, the tempering device can have all of the advantages and features described in this context.

Preferably, the lower limitation of the second receiving area has a through hole.

The through-hole is designed so that an outlet of the second container can be passed through the through-hole. In particular, when the second container is inserted through the open top into the second receiving area and received, an outlet at a lower end of the second container can be guided through the through-hole, so that the outlet of the second container in the state received in the second receiving area through the through-hole is guided and protrudes below the second exception area. This enables the outlet to be connected to the preparation device, so that a correctly dosed amount of fluid can exit by means of the second container and can be mixed with the baby food concentrate or coffee powder also supplied to the preparation device and in a container, preferably in a baby bottle or in a filter container and/or in a coffee cup or coffee pot can be filled. However, it is also conceivable that the correctly dosed quantities of fluid and baby food concentrate or coffee powder are fed to a container or a baby bottle or a coffee container or a coffee cup or coffee pot. The ready-to-eat baby food or coffee can then be produced by shaking or jiggling the container or the baby bottle or the coffee container or the coffee cup or coffee pot. In other words, the correctly dosed quantities of fluid and baby food concentrate or coffee powder are mixed by shaking or jiggling the container or the baby bottle or the coffee container or the coffee cup. Shaking or jiggling can be done manually by the user. However it is also conceivable that the device has a shaking device and/or a mixing device by means of which the correctly dosed fluid and baby food concentrate or coffee powder in the container or in the baby bottle or in the coffee container are vibrated and/or mixed. It is also conceivable that instead of shaking or jiggling 3D acoustic waves are used.

Preferably, a container for receiving and dosing fluid (in particular liquid) for preparing baby food or coffee is provided, the container having a housing with an interior space for receiving fluid, an inlet in fluid connection with the interior space and an outlet in fluid connection with the interior space includes. Furthermore, the inlet can be connected to an outlet of a fluid reservoir and a dosing of the fluid for the preparation of baby food or coffee can be dispensed through the outlet of the container. The container is replaceable and designed as a single-use-article.

Preferably, the container for receiving and dosing fluid (in particular liquid) can be supplied pre-filled with fluid. The container can be supplied filled with the fluid from the factory, i.e. the container can be filled with fluid at the factory so that the container can already be supplied to the consumer filled with fluid for the preparation of coffee or baby food.

The fluid reservoir can be replaceable, i.e. the fluid reservoir can be designed as a disposable or single-use articles, just as the first container for baby food concentrate or coffee powder, the dosing device for dosing the baby food concentrate or coffee powder, the second container for a fluid and the preparation device can be designed as replaceable components. However, it is also conceivable that the respective components previously described as replaceable are designed as reusable or reusable components. The fluid reservoir can be connected to the second container in such a way that the device for preparing baby food, in particular the second receiving area, does not come into contact with the fluid. Thus, the device, in particular the second receiving area, is not contaminated with fluid, so that cleaning of the device is not necessary after each individual preparation of baby food or coffee.

Preferably, the container is designed to be introduced into and received by a device for preparing baby food or coffee as a second container.

The container can be designed to be introduced as a second container into the second receiving area of the previously described device for preparing baby food or coffee and at least partially to be received therein. Thus, all the previously described features of the device that were described in connection with the second container also apply to the second container described below for receiving and dosing fluid. In particular, the second container described below can be inserted and received in the second receiving area of the device as previously described, so that precise dosing of the fluid for preparing baby food or coffee is made possible by means of the clamping elements of the individual clamping element pairs.

Preferably, the inlet of the second container comprises an inlet opening, which is preferably arranged essentially opposite the outlet of the second container viewed in the direction of a container longitudinal axis and/or essentially opposite an outlet opening in the outlet of the second container viewed in the direction of the container longitudinal axis. The second container may comprise an inlet with an inlet opening and an outlet with an outlet opening, the outlet being arranged on a side opposite the inlet. When the second container is introduced into the second receiving area by a essentially vertical movement through the open top, the second container is received by the second receiving area in such a way that the outlet is arranged in a lower area of the second receiving area, adjoining or near to the first, lower pair of clamping elements and adjoining or near to the lower limitation. Thus, the outlet can be carried out through the through-hole in the lower limitation of the second receiving area. At the same time, the inlet is arranged in an upper region of the second receiving region, adjoining or near to the open upper side and adjoining or near to the second, upper pair of clamping elements. By enabling the inlet can be connectable to an outlet of a fluid reservoir, fluid can flow from the fluid reservoir into the interior of the second container are guided and by means of the clamping elements of the individual clamping element pairs, the fluid can be dosed in the desired amount, which is necessary for the preparation of baby food or coffee, and can exit the second container through the outlet. This enables a predetermined or predeterminable dosing of the fluid for preparing baby food or coffee and a correct dosing of the fluid by means of the clamping elements.

Preferably, the inlet of the second container is firmly connected to the outlet of the fluid reservoir, preferably screwed or glued.

The second container can be firmly connected to the fluid reservoir. Thus, the second container and the fluid reservoir can thus be configured as a unit that is firmly connected to each other. Preferably, the fluid reservoir can be integrated into the container so that the fluid reservoir is formed integrally with the second container. As a result, the second container and the fluid reservoir can be introduced into the receiving area and received as a unit connected to each other. By having the outlet of the fluid reservoir is connected to the inlet of the second container, after the second container is received in the second receiving area, the fluid can be guided from the fluid reservoir into the interior of the second container and by means of the clamping elements in the desired amount dosed and fed through the outlet to the preparation device. This enables the fluid reservoir and the second container to be provided as a unit and the user no longer has to manually join the fluid reservoir and the second container together. The second container can thus be connected to the fluid reservoir and form a combination container or combination bag. In this condition, the combination container can be manufactured filled with fluid. In other words, the second container and the fluid reservoir can be filled with fluid. In this case it is conceivable here that only the fluid reservoir is filled with fluid and the second container or the dosing and sterilization area is arranged or fastened to the fluid reservoir when folded. The fluid reservoir filled with fluid, for example a Tetra-Pak, can be separated from the second container with a separating element, for example with a clamp. By using a bracket as a separating element prevents the fluid from flowing from the fluid reservoir into the second container and from being able to escape from the outlet of the container when the second container is opened.

However, it is also conceivable that the fluid reservoir and the second container are two separate elements which are provided separately from each other. Thus, the inlet of the second container can first be connected to the outlet of the fluid reservoir, for example by means of a screw connection or by means of a plug connection or by means of an adhesive connection or by means of a clamp connection, so that the second container and the fluid reservoir then together through the open top into the second receiving area can be introduced.

The second container can be made of different materials and can include, for example, plastic or other flexible materials, for example a film material, which are suitable for receiving a fluid. Furthermore, the second container can be designed as a bag or a pouch. Like the second container, the fluid reservoir can be formed from a flexible material. However, it is also conceivable that the fluid reservoir is formed from a non-flexible material and is thus dimensionally stable, wherein the fluid reservoir can comprise a metal such as aluminum or a plastic, for example. For example, the fluid reservoir can also be designed as a cardboard box, for example a Tetra Pak. The fluid reservoir and the second container are preferably made from the same material, in particular when the fluid reservoir and the second container are made as a unit and not as two separate elements.

Preferably, the second container comprises a essentially horizontal plate which is arranged adjoining or near to the inlet opening and/or adjoining or near to the inlet of the second container. Preferably, the plate is connectable to the second container or the plate is firmly connected to the second container or the plate is integrated into the second container. The horizontal plate can also be integrated into the fluid reservoir.

The plate or suspension strap can be connected to the upper region of the second container in a firmly or detachable manner. The plate can be integrally formed with the second container. Preferably, the plate has a surface shape which essentially corresponds to the surface shape of a cross-section of the second container at an angle different from 0° or 180°, preferably at an angle of 90°, in particular seen transversely to the longitudinal axis of the second container. The surface shape of the plate can be, for example, rectangular or square or circular or oval. However, other forms are also conceivable. The distance between two opposite sides of the surface shape of the cross section of the plate is preferably equal to or greater than the distance between two opposite side surfaces of the second container when it is inserted and received in the second receiving area or when it is filled with a fluid or when inside the second container a fluid is added.

The plate enables a simplified introduction of the second container into the second receiving area and a subsequent holding or positioning of the second container in the second receiving area. In the state of the second container received in the second receiving area, the plate rests on the edges or marginal surfaces of the open top side, so that the plate covers the open top side. In addition, the plate enables precise insertion of the second container into the second receiving area, so that the clamping elements of the individual clamping element pairs can exert pressure on the side walls of the second container and the tempering device can come into contact with the second container. This enables precise tempering of fluid to the desired temperature and subsequent dosing for the preparation of baby food.

As an alternative to the plate, a positioning and holding device or suspension device can be provided, which essentially fulfills a similar purpose as the plate. The positioning and holding device is preferably a clamp or a C-clamp or a C-holding element with a C-shape educated. This C-clamp can be arranged between the second container and the fluid reservoir, preferably at the point at which the second container is connected to the fluid reservoir in the case of the combination container. The C-clamp can, for example, be fastened, preferably glued, to the underside of the fluid reservoir or to the top of the second container. It is also conceivable for the positioning and holding device to comprises an adhesive element, for example an adhesive strip, and/or a Velcro element instead of the C-clamp or the C-holding element.

The combination container can be positioned and held by means of the positioning and holding device on one of the side walls of the second receiving area, preferably in an upper area of the second receiving area. The positioning and holding device prevents the combination container, in the state inserted into the second receiving area, from sliding downwards in the direction of the lower limitation while the fluid is being emptied. This ensures that the second container can empty completely. The positioning and holding device is designed to hold the second container and/or the fluid reservoir in position.

Preferably, the plate has a through-hole, the plate preferably comprising a first flange with a first peripheral wall, the first peripheral wall at least partially surrounding the through-hole and extending at an angle different from 0° or 180°, in particular essentially transversely from a first Side of the plate extends away. The first flange is preferably designed to connect the plate to the outlet and/or to an outlet opening of the fluid reservoir.

The first peripheral wall of the first flange of the plate is designed to be able to engage with the fluid reservoir, in particular the outlet of the fluid reservoir. This enables the second container to be fluidly connected to the fluid reservoir, so that the fluid can be reliably introduced from the fluid reservoir into the second container. The first flange or the first the peripheral wall can be made in one piece with the plate, or it can be made as a cast part or injection-molded part that can be connected to the plate. The outer wall of the first flange can be shaped essentially round and the outer wall of the outlet of the fluid reservoir can be shaped essentially round. However, other shapes are also conceivable, for example an oval shape.

The first flange can be connected to the outlet of the fluid reservoir, for example by means of a plug connection. Accordingly, the inner diameter of the first flange or the first peripheral wall can essentially correspond to the outer diameter of the outlet of the fluid reservoir, or the inner diameter of the first flange or the first peripheral wall can be slightly larger than the outer diameter of the outlet of the fluid reservoir. The outlet of the fluid reservoir can thus be connected to the first flange in a simple manner, so that fluid can be reliably introduced into the second container. However, it is also conceivable that the first flange can be connected to the outlet of the fluid reservoir by means of a screw connection. Thus, the first peripheral wall of the first flange can comprise a first thread, for example on the inside or on the outside of the first peripheral wall relative to the through-hole, which is connected to a second thread of the outlet of the fluid reservoir, for example on the outside or on the inside of a peripheral wall of the outlet, can be screwed.

Thus, the second container can be connected to the fluid reservoir by simply plugging it on or screwing it. However, it is also conceivable that the second container is glued to the outlet of the fluid reservoir by means of the first flange or that the second container is formed integrally with the fluid reservoir, for example as a combination container. A tank, which can be provided to the dimensions of the device for preparation, can be provided as the fluid reservoir. For example, the tank can have a cross-section in the plane of one of its side surfaces in which the outlet and the outlet opening are arranged, which is essentially the cross-section of the device for preparing baby food or coffee, viewed at an angle different from 0° or 180°, in particular transversely, to the longitudinal axis of the device or to the longitudinal axis of the second receiving area. However, it is also conceivable that the fluid reservoir is a bottle in which fluid suitable for the preparation of baby food or coffee is received and which can be purchased, for example, in the supermarket. Instead of the bottle, a container, in particular a Tetra Pak, is also conceivable. In this case, the bottle opening or the opening of the Tetra Pak can be screwed as an outlet to the first flange in a simple manner, for example the external thread of the bottle opening or the opening of the Tetra Pak with the first thread, for example on the inside of the first flange or the first peripheral wall, can be screwed.

Preferably, the plate preferably has a second flange with a second peripheral wall, the second peripheral wall at least partially surrounding the through-hole and extending essentially transversely away from a second side of the plate opposite the first side. Preferably, the second flange is designed to connect the plate to the inlet and/or to the inlet opening of the container.

As previously described, the plate can be firmly connected to the second container, in particular a second side of the plate, which is opposite the first side with the first flange and the first peripheral wall, can be firmly connected to the second container, so that the second container by means of the plate is connectable to the fluid reservoir. However, it is also conceivable that the plate is a separate element which can be connected to the outlet of the fluid reservoir by means of the first flange and which can be connected to the inlet of the second container by means of a second flange.

The second flange comprises a second peripheral wall and is arranged on the second side of the plate in such a way that the second flange and the second peripheral wall at least partially surround the through-hole. The second flange and the second peripheral wall can be essentially the same as the first flange and the first peripheral wall be designed. The first flange and the second flange preferably surround the same flange center longitudinal axis, which extends at an angle different from 0° or 180°, in particular transversely to the plane of the plate and/or through the through opening. Thus, the plate can be connected to the fluid reservoir by means of the first flange and to the inlet of the second container by means of the second flange, a fluid reservoir central longitudinal axis of the fluid reservoir and the container longitudinal axis of the second container running in the same straight line with the flange central longitudinal axis if the fluid reservoir, the plate and the second container are connected to each other. The fluid reservoir central longitudinal axis extends through the outlet, so that the outlet opening is arranged around the fluid reservoir central longitudinal axis. The longitudinal axis of the container extends through the inlet so that the inlet opening is arranged around the longitudinal axis of the container. The outlet opening and/or the outlet of the second container can also be arranged around the longitudinal axis of the container. It is also conceivable, however, that the outlet opening and/or the outlet do not extend around the longitudinal axis of the container, but rather around a longitudinal axis which extends in a plane parallel to the longitudinal axis of the container.

It is also conceivable that the inlet of the second container can be connected directly to the outlet of the fluid reservoir. In this way, the plate can be dispensed with. Preferably, the outlet of the fluid reservoir can be connectable to the inlet of the second container by means of a plug connection or by means of a screw connection. The inlet of the second container can, however, also be glued to the outlet of the fluid reservoir, or can be connected to each other in one piece. The inlet of the second container preferably comprises the first thread, for example on the inside or on the outside of the inlet, viewed relative to the inlet opening, the first thread with the second thread at the outlet of the fluid reservoir, for example on its inside or outside, viewed relative to the outlet opening, can be screwed. The fluid reservoir preferably comprises a housing with an upper side and a lower side, the upper side and the lower side being arranged at opposite ends to the fluid reservoir central longitudinal axis. The outlet of the fluid reservoir is arranged on the underside, the underside running in a essentially horizontal plane or in a plane at an angle different from 0° or 180°, in particular at an angle of 90°, seen transversely to the fluid reservoir central longitudinal axis. With this configuration, the underside can assume the function of the plate.

The fluid reservoir can also have an inlet having an inlet opening, which is preferably arranged opposite the outlet or the outlet opening. In this way, a fluid can be introduced into the fluid reservoir through the inlet and/or added. However, it is also conceivable that the fluid reservoir does not comprise an inlet or an inlet opening, in particular if the combination container filled with fluid is produced.

Preferably, the outlet of the container is for insertion into a through-hole configured in the lower limitation of the second receiving area.

The outlet of the second container can be designed as an elongated element, for example the outlet can be tubular and the outlet can extend between a first end and an opposite second end along a longitudinal axis of the outlet. The first end is disposed adjoining or near to the second container and the second end is spaced from the second container. The outlet comprises an outer diameter which is smaller than the inner diameter of the through-hole in the lower limitation of the second receiving area. Thus, the outlet of the second container can be guided through the through-hole in the lower limitation of the second receiving area when the second container is inserted into the second receiving area. This enables the outlet to be connected to the preparation device when the second container is inserted into the second receiving area, so that a correctly dosed amount of fluid can emerge from the second container and, by means of the preparation device, with that which is also supplied to the preparation device baby food concentrate or coffee powder can be mixed and filled into a container, preferably into a baby bottle or into a filter and/or funnel container and/or into a coffee cup or coffee pot. It is also conceivable that in this way the fluid and the baby food concentrate or coffee powder are introduced or dosed directly into the container or the baby bottle or the coffee cup, so that the preparation device can be dispensed with. The first end of the outlet can be firmly connected to the second container, for example the first end of the outlet can be designed in one piece with the second container or it can be glued to the second container. When the second container is in the state of the second container being received in the second receiving area, the second end of the outlet can lie in a plane which lies below the plane of the lower limitation. However, it is also conceivable that the second end lies in the same plane as that of the lower limitation, or in a plane adjoining or near to the plane of the lower limitation, for example above the level of the lower limitation.

Preferably, the second container has at least partially a tapering section, wherein the periphery of the second container in the tapering section decreases towards the outlet, preferably essentially conically.

The second container can extend between an inlet end and an opposite outlet end along the longitudinal axis of the container. Adjoined or near to the inlet end, the inlet opening and the inlet are arranged. Adjoined or near to the outlet end, the outlet and the outlet opening are arranged. The second container can have a first side wall and an opposing second side wall which extend essentially parallel to the plane of the longitudinal axis of the container between the inlet end and the outlet end. In the lower area near to the outlet end, the container has a tapering section. In the tapered section, the distance between the first side wall and the second side wall decreases towards the outlet, preferably essentially conically. This enables the fluid to be guided almost completely out of the second container through the outlet, so that a minimal residual fluid remains in the second container.

Preferably, the second container has at least partially a essentially symmetrical section, the periphery of the second container remaining the same within the essentially symmetrical section and wherein the essentially symmetrical section is spaced further from the outlet than the tapered section.

In the essentially symmetrical section, the first side wall and the second side wall each extend in a plane parallel to the plane of the longitudinal axis of the container. The essentially symmetrical section can extend between the inlet end and the tapering section; when the second container is inserted or received in the second receiving area, the clamping elements of the individual clamping element pairs adjoin the side walls in the essentially symmetrical section and can apply pressure exercise the side walls. This enables dosing the fluid for preparing baby food. The first, lower pair of clamping elements is preferably arranged on the side walls of the second receiving area in such a way that it can exert pressure in a region of the second container which lies within the essentially symmetrical section and is adjoining or near to the tapered section.

Preferably, the second container comprises at least one first magnet, wherein the at least one first magnet is preferably arranged on an outer wall of the tapered portion, and wherein the at least one first magnet with at least one second magnet adjoining or near to the through-hole in the lower limitation of the second receiving area is connectable.

The at least one first magnet can be arranged adjoining or near to the outlet, preferably on an outer wall of the outlet. The at least one second magnet can be adjoining or near to the be arranged through hole in the lower limitation, preferably on an inner wall of the through-hole. The at least one first magnet can at least partially surround the outer wall of the outlet, preferably the at least one first magnet can completely surround the outer wall of the outlet. The at least one second magnet can at least partially surround the inner wall of the through hole, preferably the at least one second magnet can completely surround the inner wall of the outlet. The at least one first magnet and the at least one second magnet are preferably arranged in such a way that they can interact when the second container is received in the second receiving area. In this way, the second container is brought or held in a suitable position so that the fluid can be guided almost completely out of the second container through the outlet and/or so that the individual clamping elements enable or ensure optimal dosing of the fluid. Alternatively, it is also conceivable that, instead of the at least one first magnet, a metal element (or a metal plate or a metal strip) is provided which interacts with the second magnet. It is also conceivable that instead of the at least one second magnet, a metal element (or a metal plate or a metal strip) is provided which interacts with the first magnet. Due to the magnets, the second container is always in the correct position, so that the fluid for preparing baby food or coffee can be guided out of the outlet without the fluid reaching the housing of the second receiving area.

Preferably, the second container is a hose or the second container is designed as a hose.

Preferably, the fluid in the interior of the second container can preferably be dosed by means of a peristaltic pump.

Preferably, the second container or the hose and/or the peristaltic pump can be introduced into the second receiving area of the device for dosing and/or preparing baby food, in particular baby milk or baby food, or coffee and can be received by the second receiving area.

Preferably, the hose and/or the peristaltic pump and/or the fluid reservoir are exchangeable and designed as single-use or disposable articles.

Preferably, the hose and the fluid reservoir are connectable or connected to each other.

Preferably, a tempering device, for example a heating plate and/or a cooling plate, is arranged adjoining to or near to the fluid reservoir. The tempering device is also preferably in contact with the fluid reservoir.

Preferably, at least one clamping element is arranged adjoining or near to the fluid reservoir. The at least one clamping element is preferably designed as a clamp. The at least one clamping element or the clamp is preferably designed to heat and/or cool at least part of the fluid in the interior of the fluid reservoir.

Preferably first container and/or the second container and/or the dosing device or the screw conveyor and/or the hose and/or the peristaltic pump are preferably made from a bioplastic or bioplastic or a bio-based plastic. The first container and/or the second container and/or the dosing device or the screw conveyor and/or the hose and/or the hose pump preferably comprise a bioplastic or bioplastic or a bio-based plastic. For example, the bioplastic can comprise stone paper and/or wood.

Preferably, the first container, for example after emptying the baby powder or coffee powder or after reaching a certain level, and/or the second container, for example after emptying the fluid or after reaching a certain level, can be automatically ordered or reordered on the Internet.

Preferably, the sensor or the scale is connected to application software, for example a mobile app, so that the level of the fluid can be automatically indicated, for example by a signal tone or a signal light, so that a new container with fluid or a new container with coffee powder or baby powder can be provided manually and/or so that a new container with fluid or a new container with coffee beans can be ordered automatically on the Internet.

Preferably, the device or the second container comprises a positioning and holding device which is designed to position and hold the second container in the second receiving area.

Preferably, device described above can preferably be operated remotely. The device can be regulated or controlled from anywhere and at any time, for example via an app on the smartphone or via remote control. In this way, baby food or coffee can be prepared remotely without anyone having to be on site near the device. Furthermore, different operating schedules are conceivable so that the device can automatically prepare baby food or coffee at a predetermined time.

A computer-implemented method for controlling or regulating the device as previously described can comprise the following steps:

Dosing the baby food concentrate or the coffee powder from the first container by means of the dosing device and/or dosing the fluid from the second container by means of a further dosing device, for example by means of the clamping elements described above or the lifting system as previously described and/or preparing the baby food or coffee by means of a preparation device (as described below), and/or determination of the fill level in the first container, which is designed to hold baby food concentrate or coffee powder, and/or determination of the fill level in the second container, which is designed to hold a fluid, and/or identification of the first component and/or the fluid and/or reordering of baby food concentrate or coffee powder and/or of fluids based on the determined level.

Preferably, a system is provided, the system comprising a device for preparing baby food, in particular baby milk or baby food, or coffee, a first container for receiving and dosing baby food concentrate or coffee powder and a second container for receiving and dosing fluid for preparing baby food.

Both the device for preparing baby food or coffee and the containers for receiving and dosing baby food concentrate or coffee powder or fluid can have all of the features as previously described and the advantages associated with these features.

The following is a brief description of the features of the preparation device, which can be provided as an optional component in the device:

First, the preparation device for using the device for dosing and/or preparing baby food is described: Preferably, the preparation device has an inner cavity which extends around a central longitudinal axis between an upper open end and a lower open end, the cavity being surrounded by an inner wall, the periphery of which preferably decreases from the upper open end to the lower open end. Preferably, the inner cavity comprises an inner wall which extends along the central longitudinal axis and divides the inner cavity into a first cavity region and a second cavity region. Preferably, a first closing flap for closing the first cavity area and a second closing flap for closing the second cavity area are arranged at the upper open end of the preparation device. Preferably includes the preparation device adjoining or near to the upper open end a connection for connecting or coupling the preparation device to the device and/or the preparation device comprises adjoining or near to the lower open end a connection for connecting or coupling the preparation device with the baby bottle.

If the device is used for dosing and/or preparing coffee, the preparation device has a filter and/or funnel container into which the coffee powder and the fluid can be introduced and/or mixed. The preparation device further comprises a container, for example a coffee cup or a coffee pot, which is arranged relative to the filter and/or funnel container in such a way that the coffee can be introduced or filled in from the filter and/or funnel container due to gravity. The coffee cup or the coffee pot is preferably arranged below the filter and/or funnel container. However, it is also conceivable that the preparation device is designed as in the dripping and cold brew process or type of preparation as in the baby dispenser. In the dripping process, the preparation unit would be placed in a vessel with a sieve at the bottom. The coffee drips from here into a container below, e.g. in a jug or vessel. In the case of the coldbrew method or type of preparation, the preparation device or stirrer and flaps of the preparation device would be in a container designed as a sieve, which is located in a further container in which fluid or water can be located.

Preferable, a drip tray is provided on the housing of the device, which drip tray extends from a side wall of the housing, preferably away from a rear wall of the housing. The drip tray is preferably arranged below the preparation device. Preferably, the distance of the first receiving area can be changed relative to the drip tray and/or the distance of the second receiving area can be changed relative to the drip tray. In particular, the housing of the device can thus be retractable or pushed together. This allows the device, for example, with a collapsed or collapsible housing, so that packaging material for transport can be saved. Furthermore, the change in the distance between the first receiving area and/or the second receiving area relative to the drip tray allows the distance to be adapted to the size of the container, in particular the baby bottle into which the baby food is to be filled, or the coffee container into which the coffee should be filled, can be adjusted. Containers of different sizes or baby bottles or coffee containers can be arranged above or on the drip tray and filled with baby food or coffee.

Preferably, the device is designed to determine the presence and/or the type of preparation device.

Preferably, the dosing device comprises a closure or flap element, the closure or flap element being designed to be opened automatically or manually, the closure or flap element preferably being designed to make the dosing device and/or the first container airtight to close.

The present invention is explained in the following on the basis of drawings showing only preferred exemplary embodiments, wherein

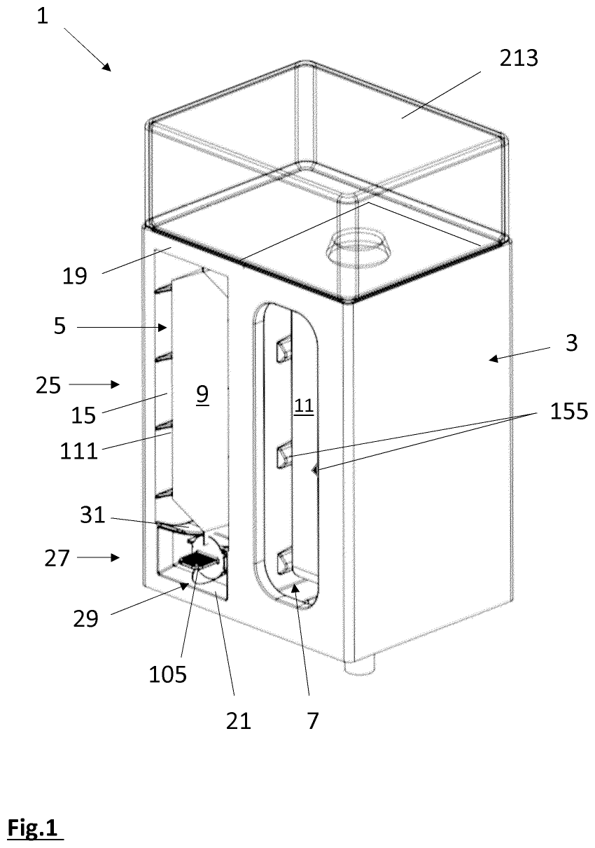

FIG. 1 shows a perspective view of an embodiment of a device for preparing baby food or coffee, in which a dosing device with a screw conveyor and a screw conveyor housing is received,

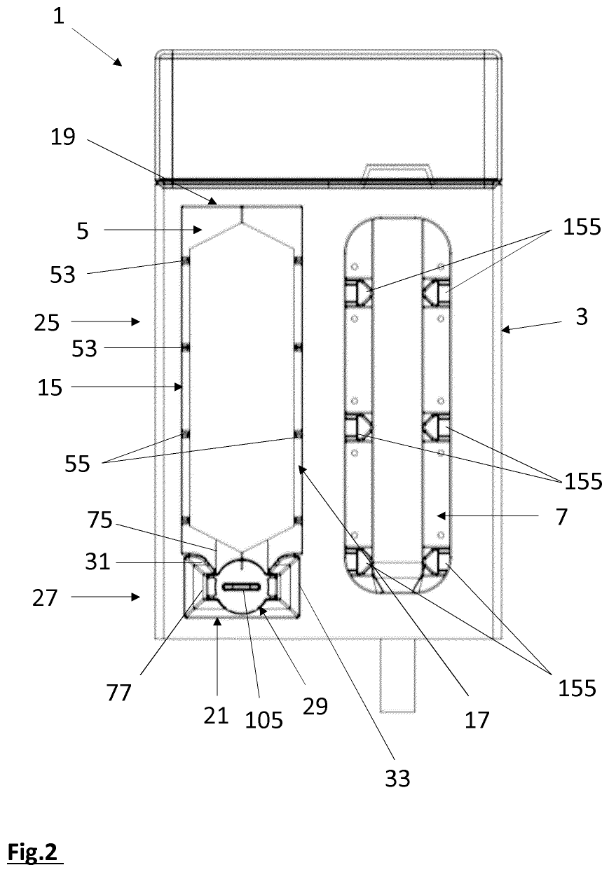

FIG. 2 shows a front view of the embodiment from FIG. 1,

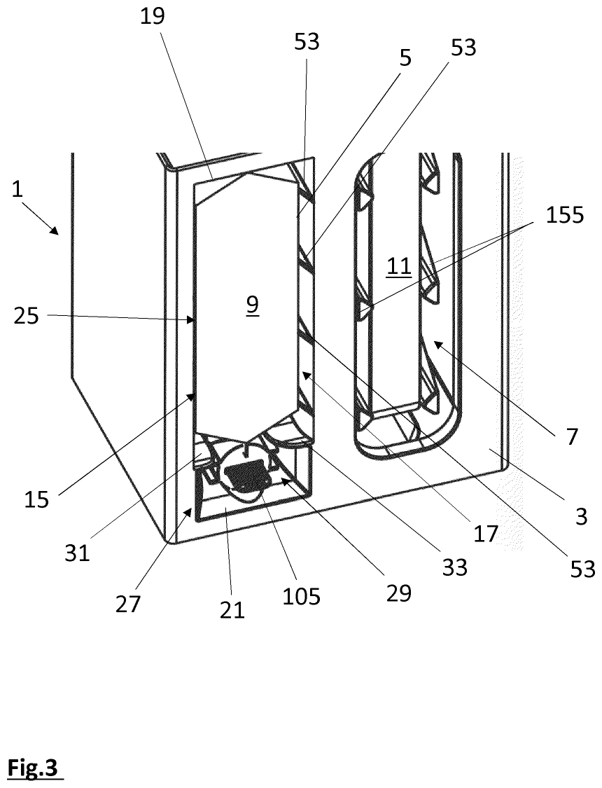

FIG. 3 shows a perspective view of a section of the exemplary embodiment from FIG. 1,

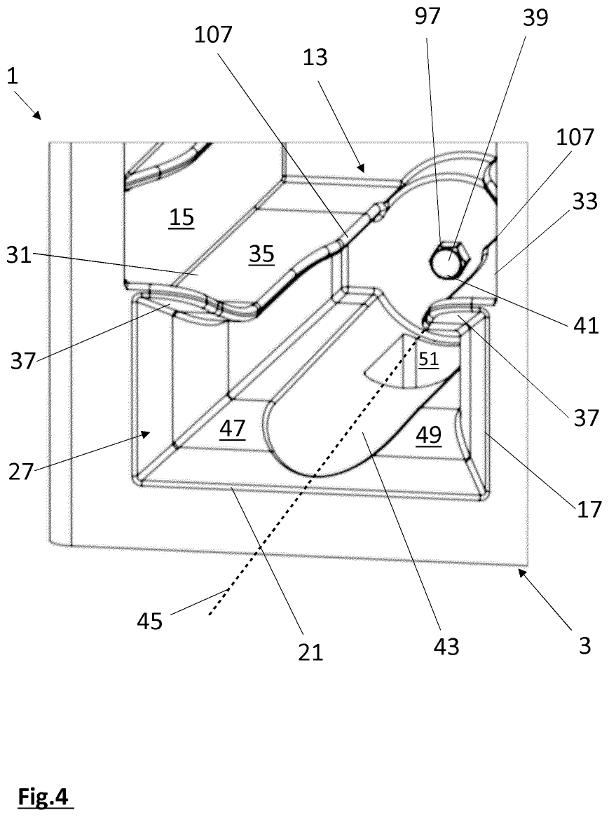

FIG. 4 shows a perspective view of the dosing device receptacle without the dosing device received,

FIG. 5 shows a perspective view of the screw conveyor,

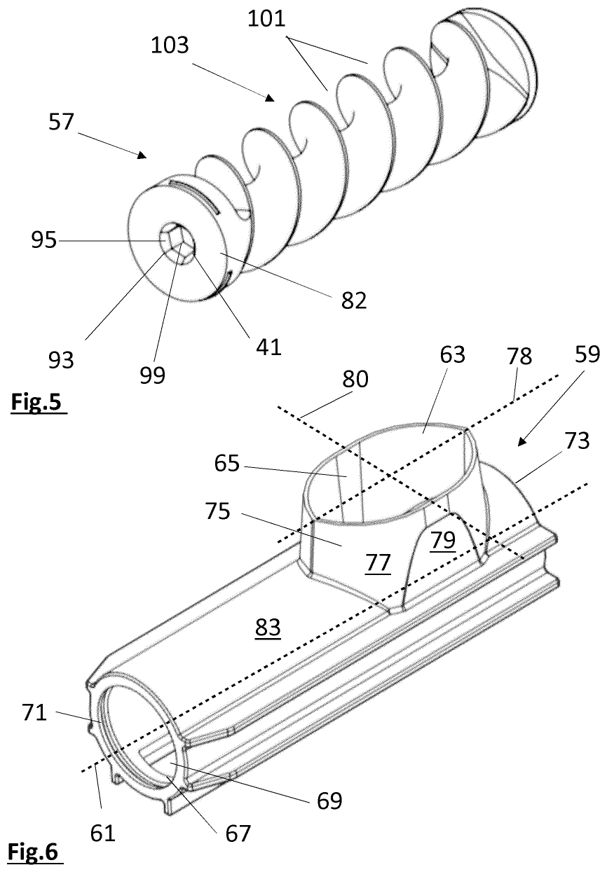

FIG. 6 shows a perspective view of the screw conveyor housing from above,

FIG. 7 shows a perspective view of the screw conveyor housing from below,

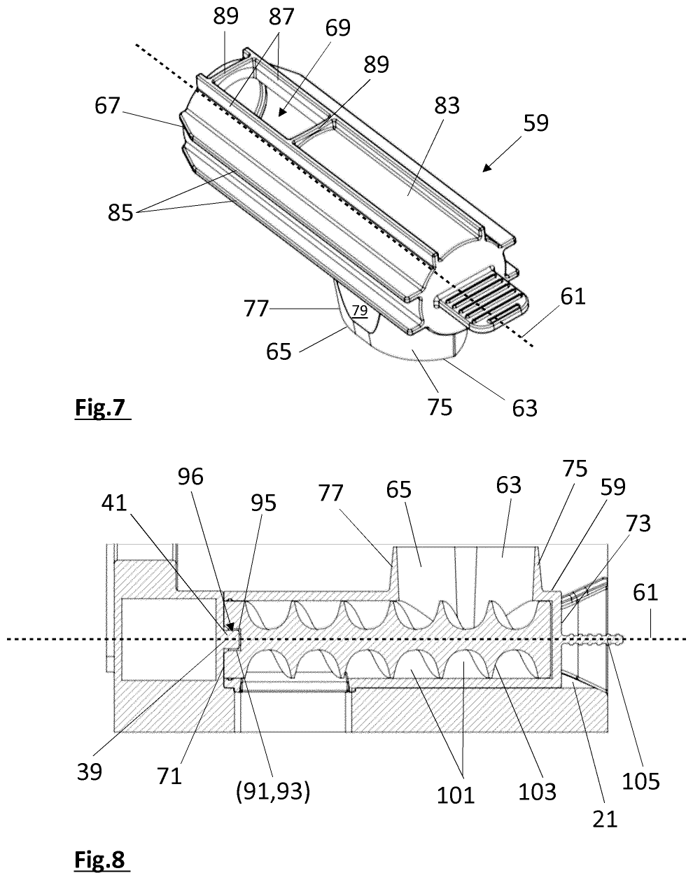

FIG. 8 shows a sectional view of the dosing device receptacle, the dosing device with the conveyor screw and the screw conveyor housing being received in the dosing device receptacle,

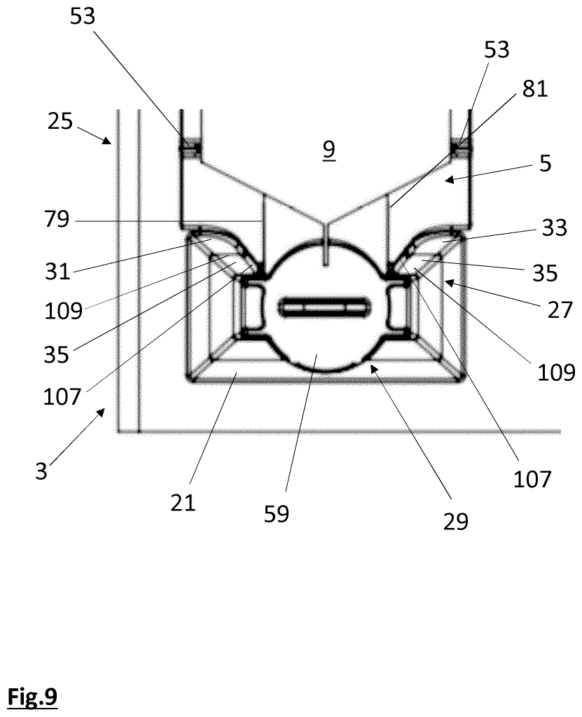

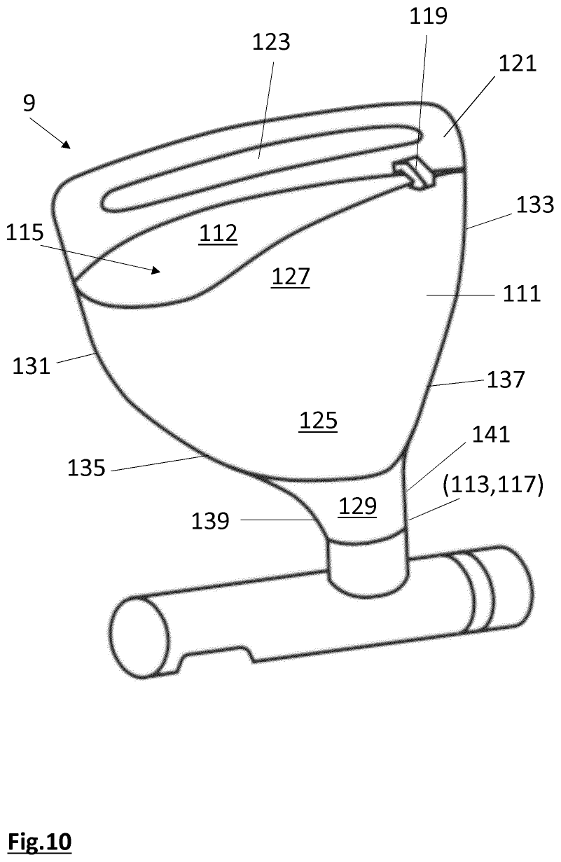

FIG. 9 shows a front view of the dosage device receptacle with the dosing device received,

FIG. 10 shows a side view of a first embodiment of a container which can be connected to a dosing device,

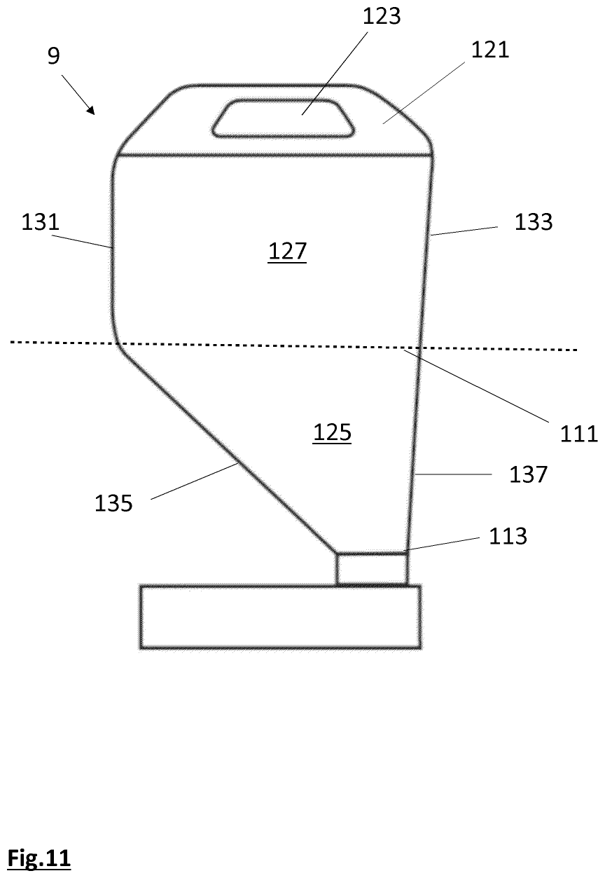

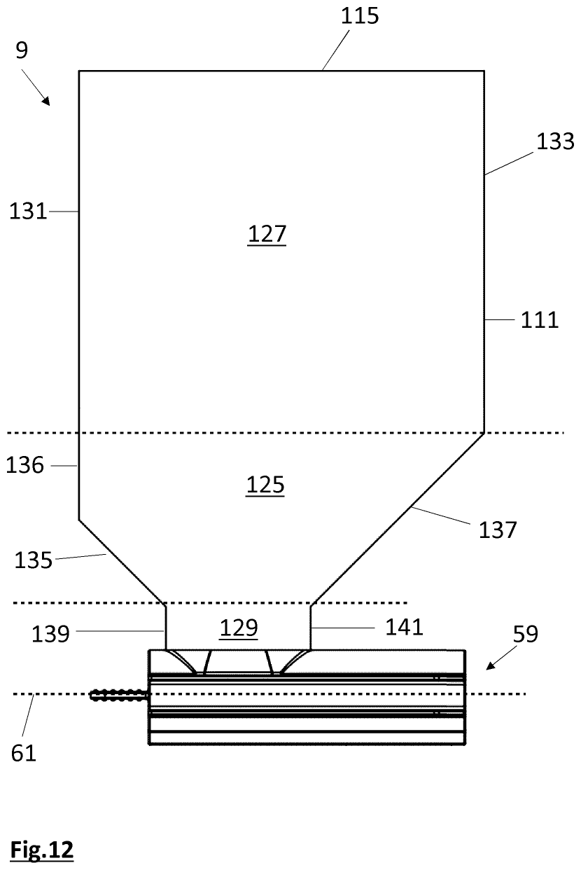

FIG. 11 shows a side view of a further exemplary embodiment of a container which can be connected to a dosing device,

FIG. 12 is a side view of a further embodiment of a container for receiving and dosing of baby food concentrate or coffee powder,

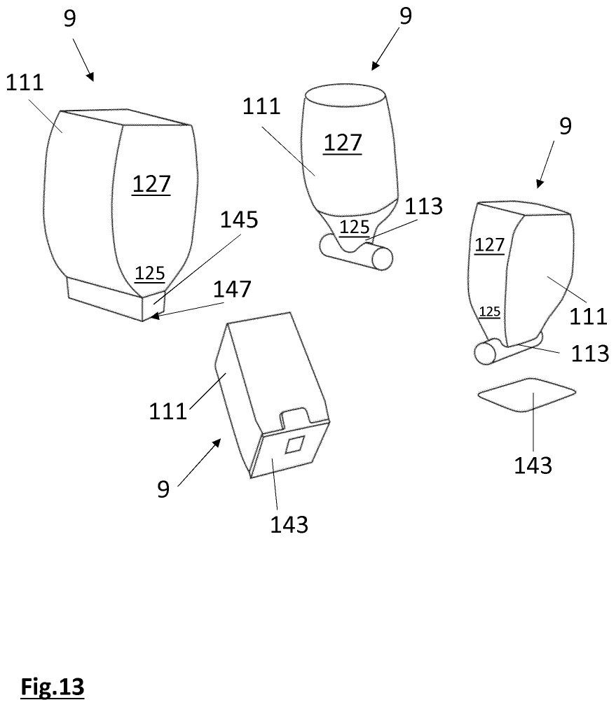

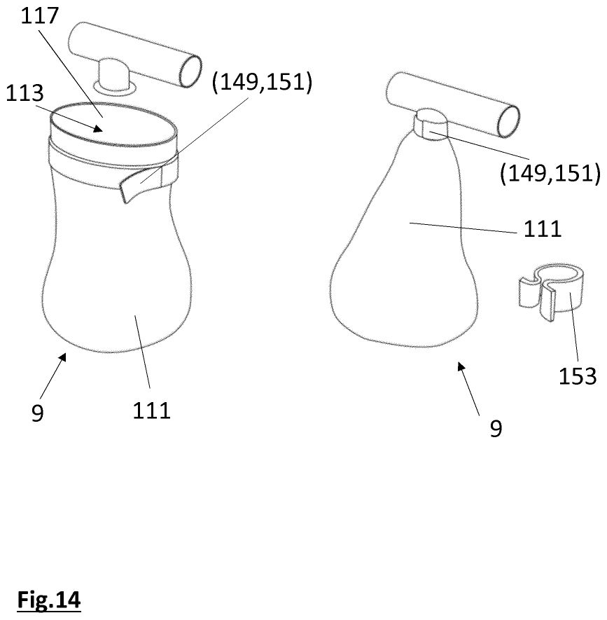

FIG. 13 shows a plurality of further exemplary embodiments of a container which can be connected to a dosing device,

FIG. 14 shows a further embodiment example of a container which can be connected to a dosing device,

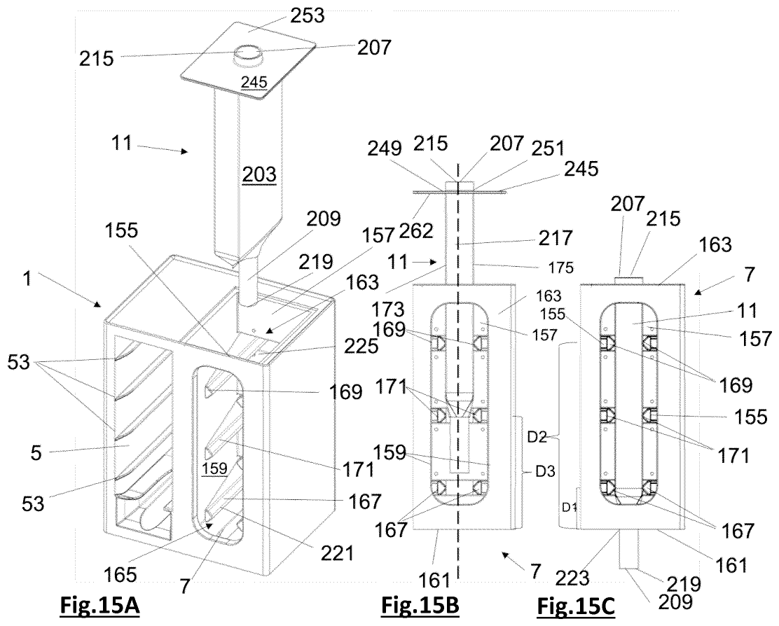

FIG. 15A shows a perspective view of the device for preparing baby food or coffee from FIG. 1, wherein the second container has not yet inserted and received in the second receiving area,

FIG. 15B shows a front view of the second receiving area of the device of FIG. 15A, the second container being inserted into the second receiving area,

FIG. 15C shows a front view of the second receiving area of the device from FIG. 15A, the second container being received in the second receiving area,

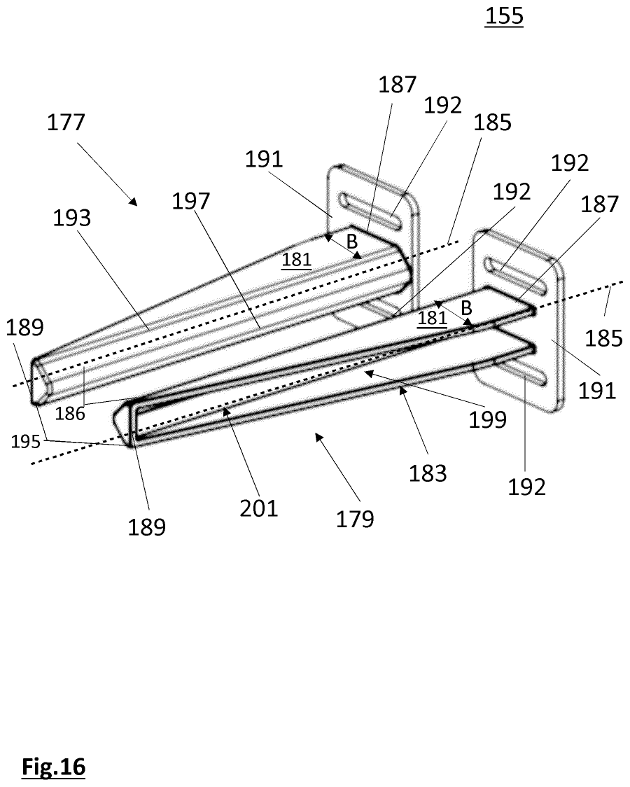

FIG. 16 shows a perspective view of a pair of clamping elements of the second receiving area,

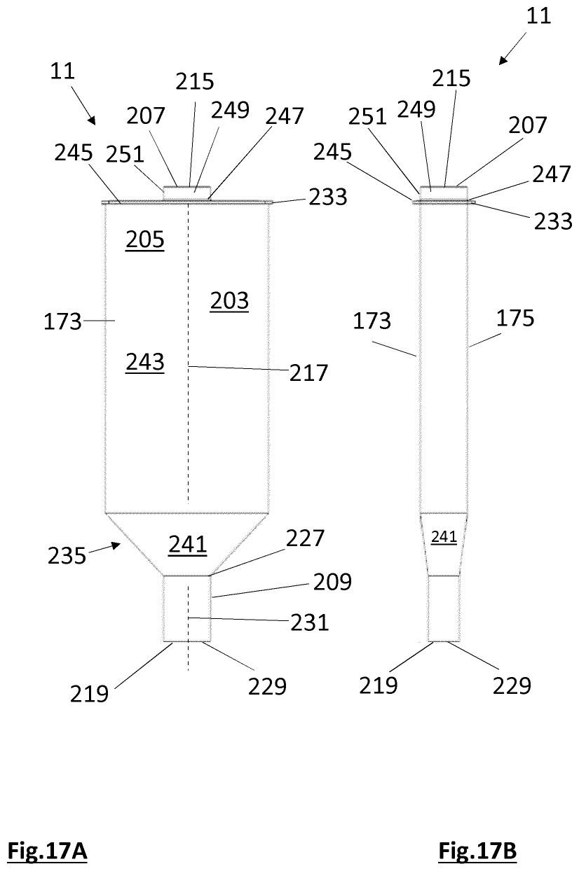

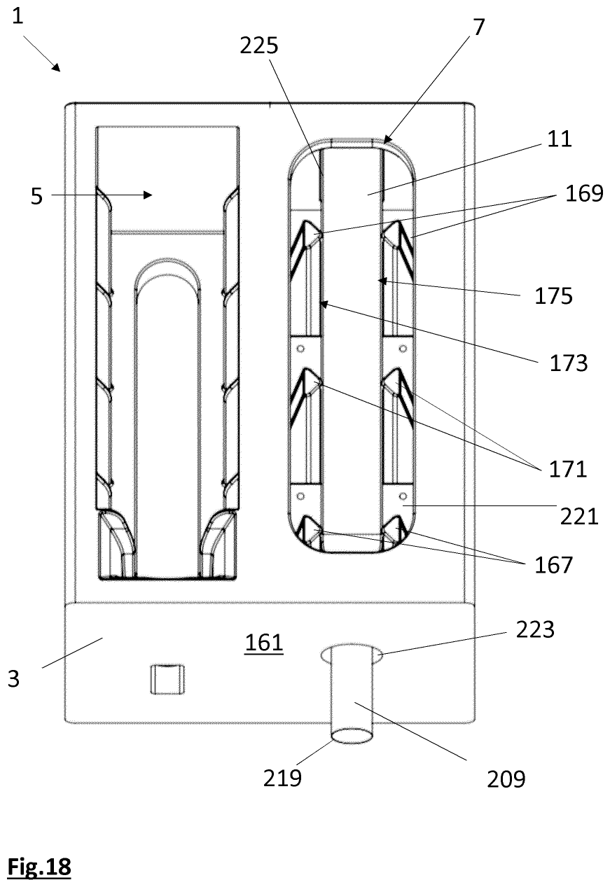

FIG. 17A shows a first side view of the second container,

FIG. 17B shows a second side view of the second container,

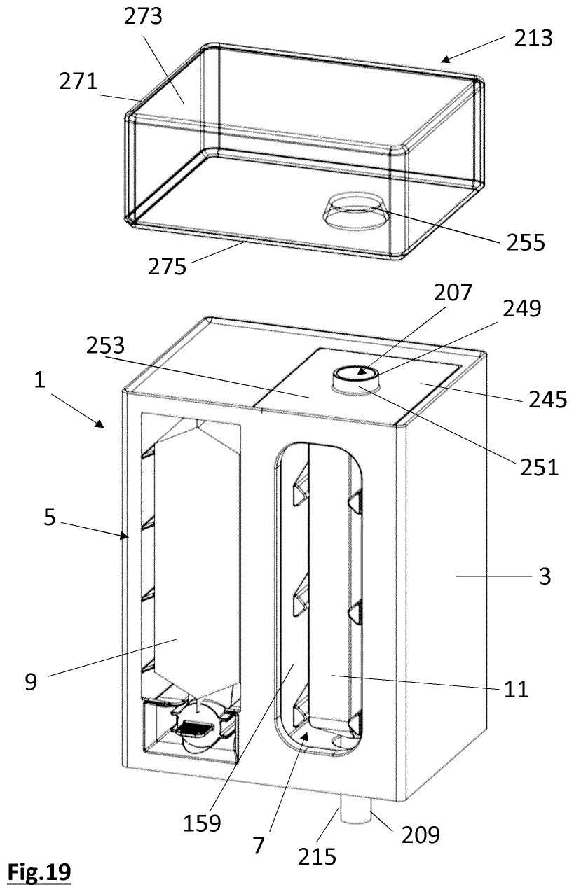

FIG. 18 shows a perspective view of the device for the preparation of baby food or coffee from below,

FIG. 19 shows a perspective view of the device for the preparation of baby food or coffee wherein the fluid reservoir is not fluidly connected to the second container,

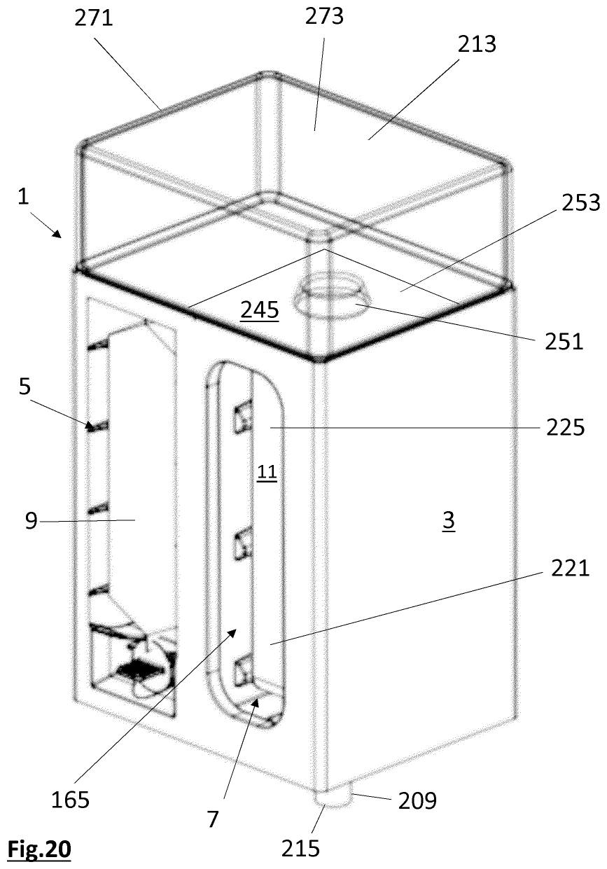

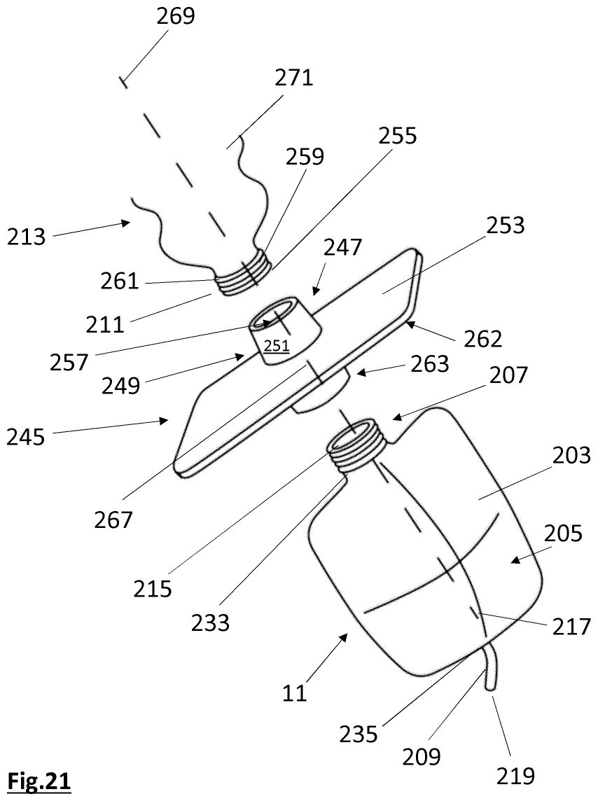

FIG. 20 shows a perspective view of the device for preparing baby food or coffee, wherein the fluid reservoir is fluidly connected to the second container,

FIG. 21 shows a perspective view of an embodiment of the fluid reservoir and the second container, which can be connected to each other by means of a essentially horizontal plate,

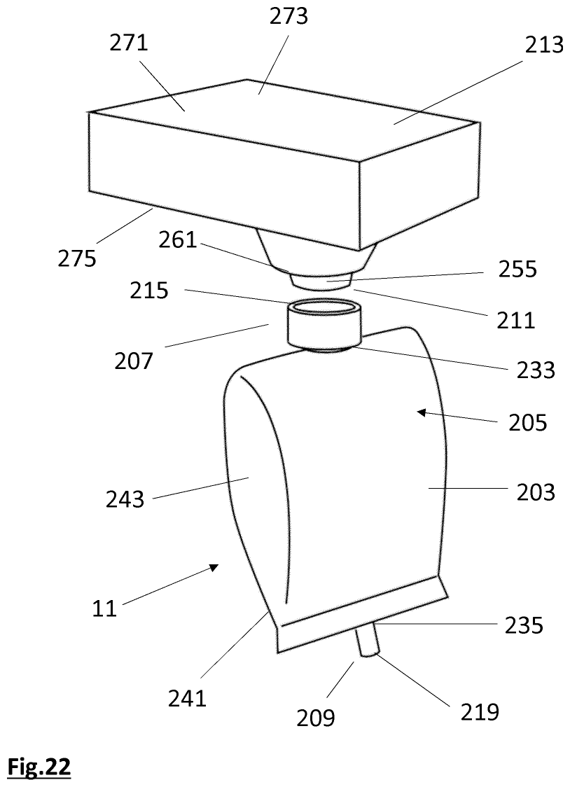

FIG. 22 shows a perspective view of a further exemplary embodiment of the fluid reservoir and the second container, wherein no essentially horizontal plate is provided for connecting the fluid reservoir and the second container,

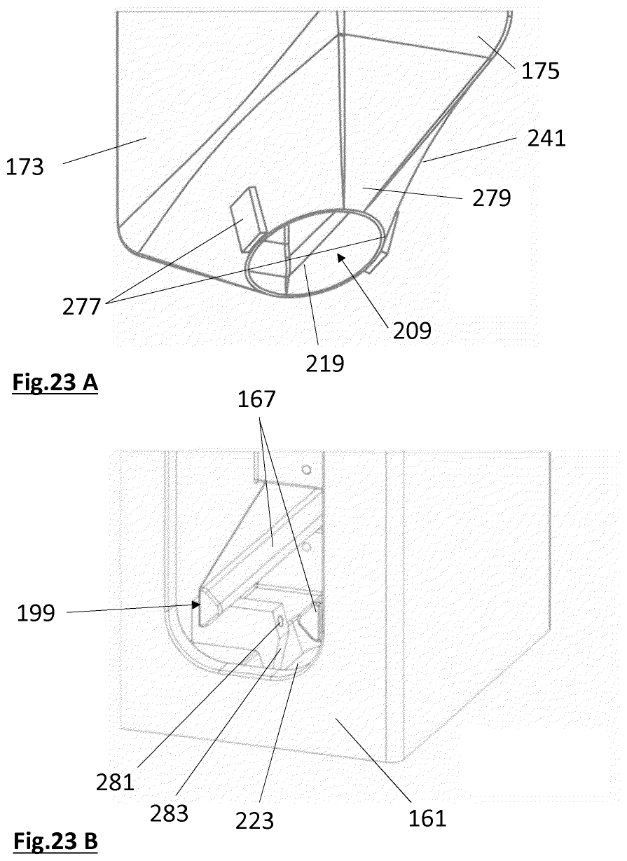

FIG. 23A shows a perspective view of the outlet of the second container,

FIG. 23B shows a perspective view of the lower area of the second receiving area,

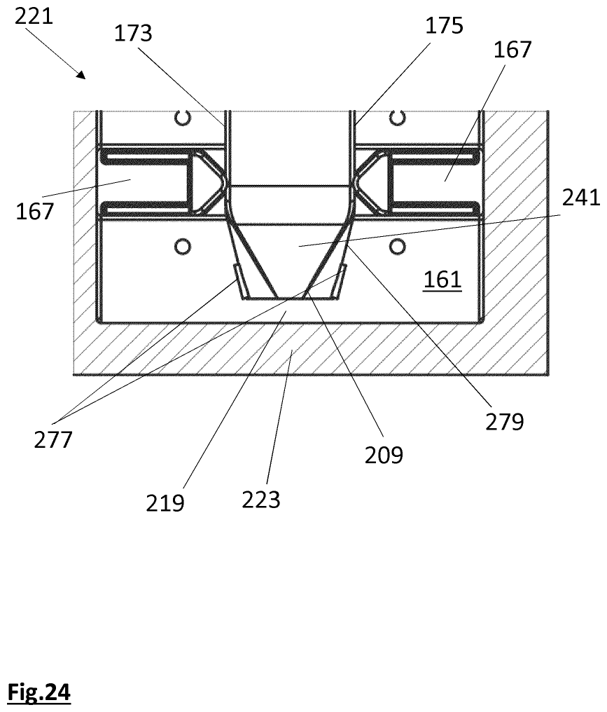

FIG. 24 shows a front view of the lower area of the second receiving area with the second container received,

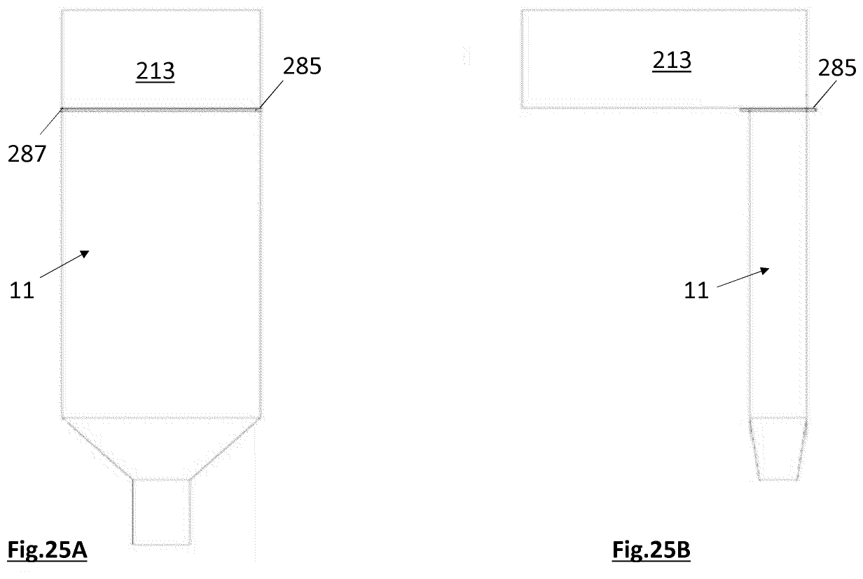

FIG. 25A shows a first side view of a combination container of the second container and the fluid reservoir,

FIG. 25B shows a second side view of the combination container from FIG. 25A,

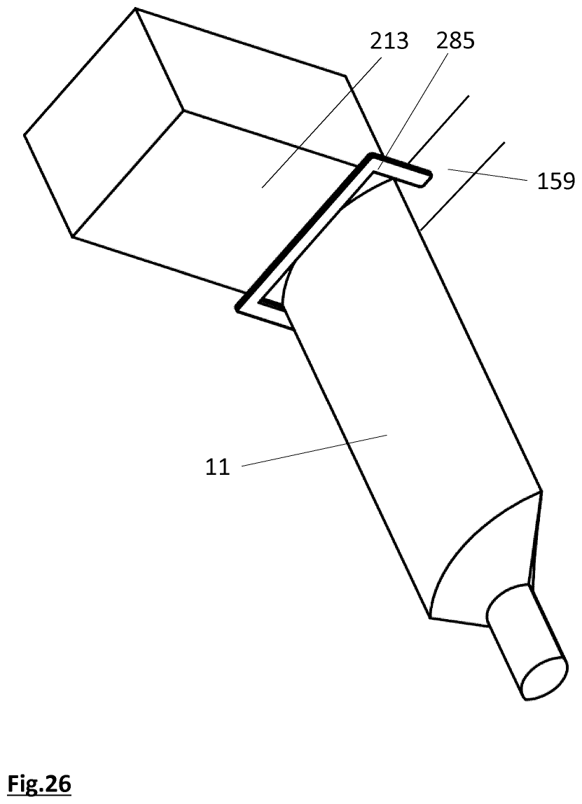

FIG. 26 shows a perspective view of the combination container from FIGS. 25A and 25B, which is positioned by means of a positioning and holding device,

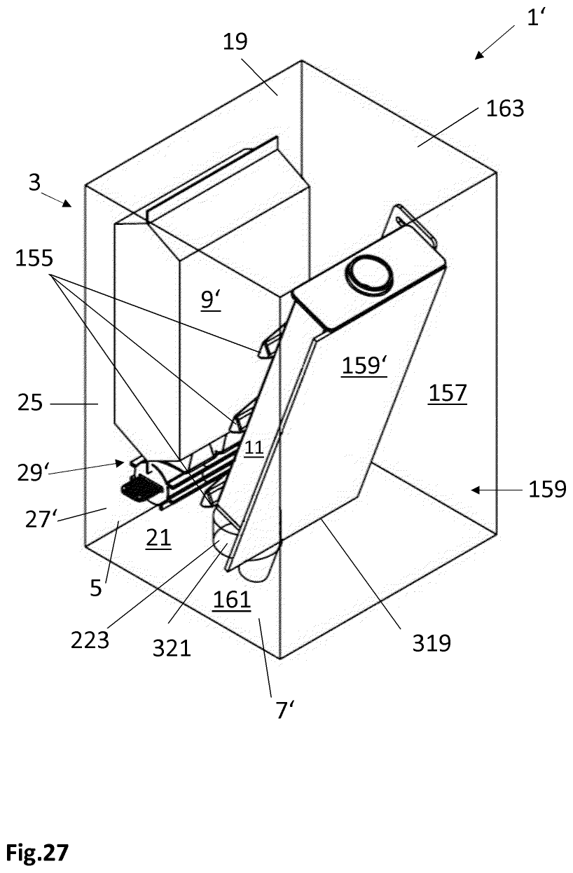

FIG. 27 shows a perspective view of an embodiment example of a device according to the invention for dosing and grinding coffee beans and/or for preparing coffee, in which a dosing and grinding device with a screw conveyor, a grinder and a screw conveyor housing is received,

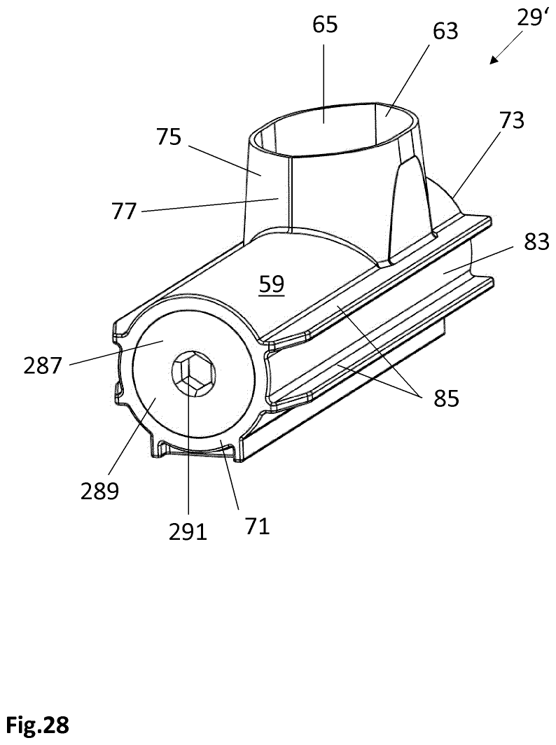

FIG. 28 shows a perspective view of the dosing and grinding device with a screw conveyor, a grinder and a screw conveyor housing,

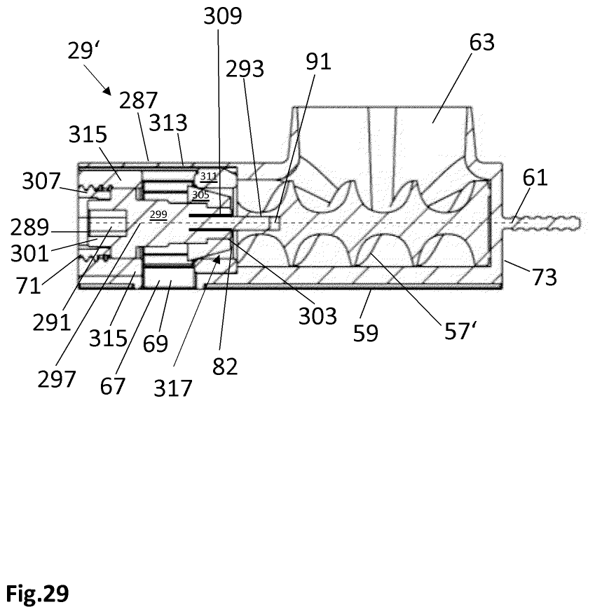

FIG. 29 shows a sectional view of the dosing and grinding device from FIG. 28, wherein the screw conveyor and the grinder being received in the screw conveyor housing of the dosing and grinding device,

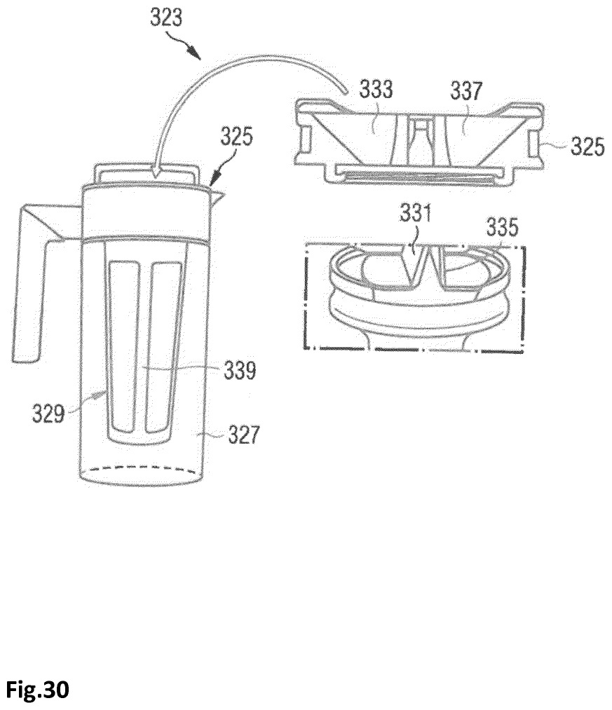

FIG. 30 shows a front view of an embodiment of a preparation device using the example of a cold brew type of preparation,

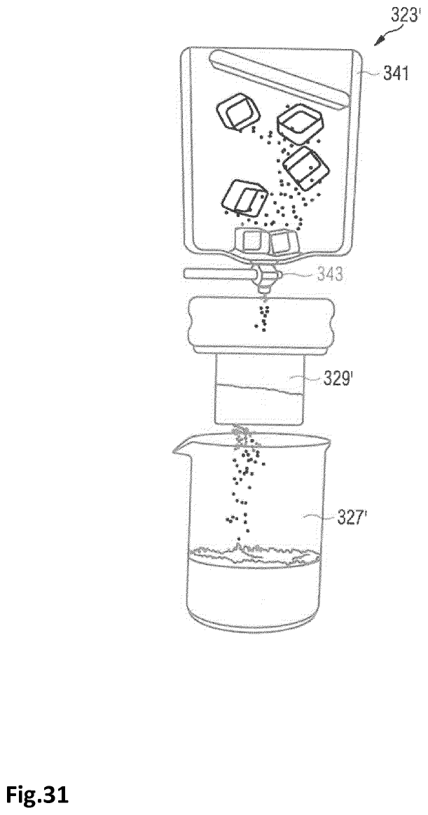

FIG. 31 shows a front view of a further embodiment of a preparation device using the example of a dripping type of preparation,

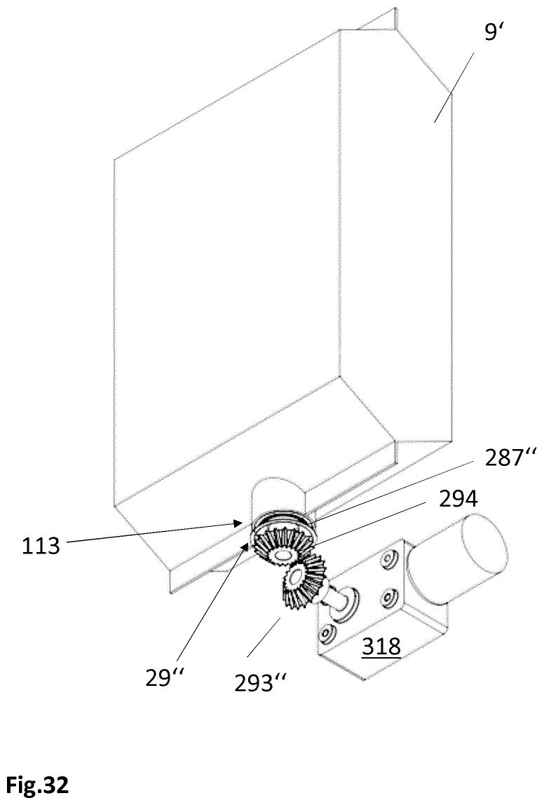

FIG. 32 shows a perspective view of a further exemplary embodiment of a first container, in which the grinding device or the grinder is rotatably arranged in the outlet of the first container,

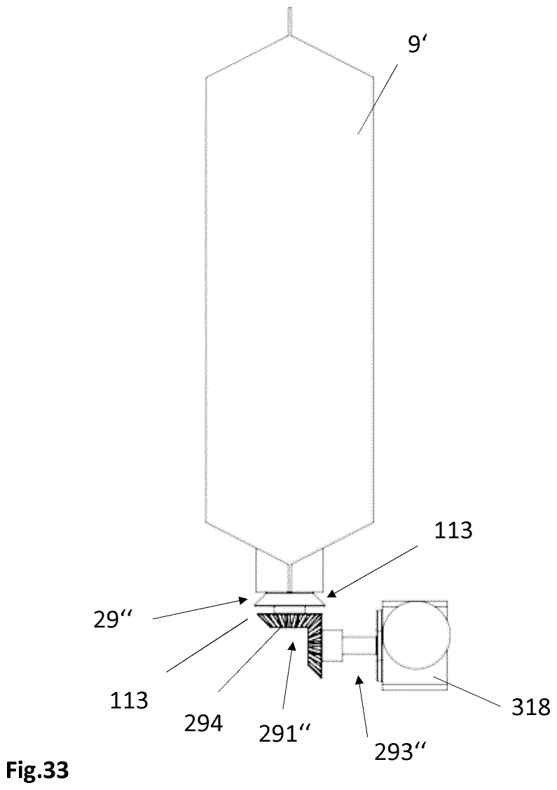

FIG. 33 shows a side view of the first container shown in FIG. 32,

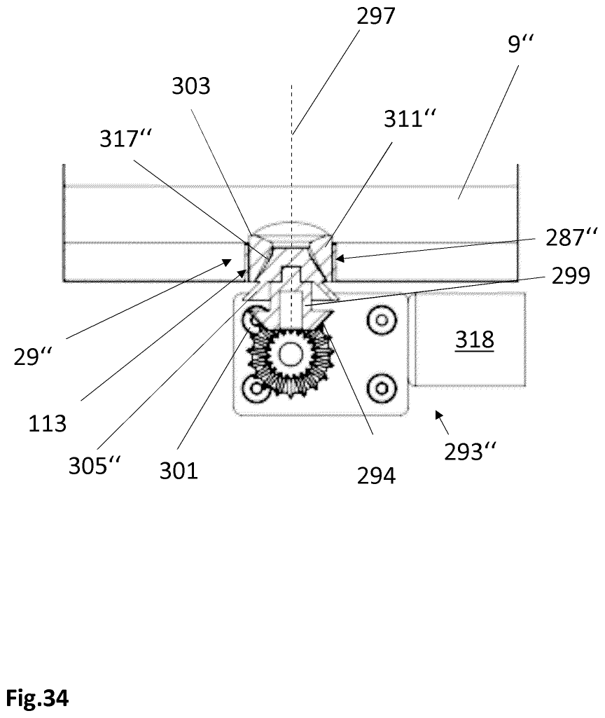

FIG. 34 is a sectional view of the lower portion of that shown in FIG. 32 first container shows, and

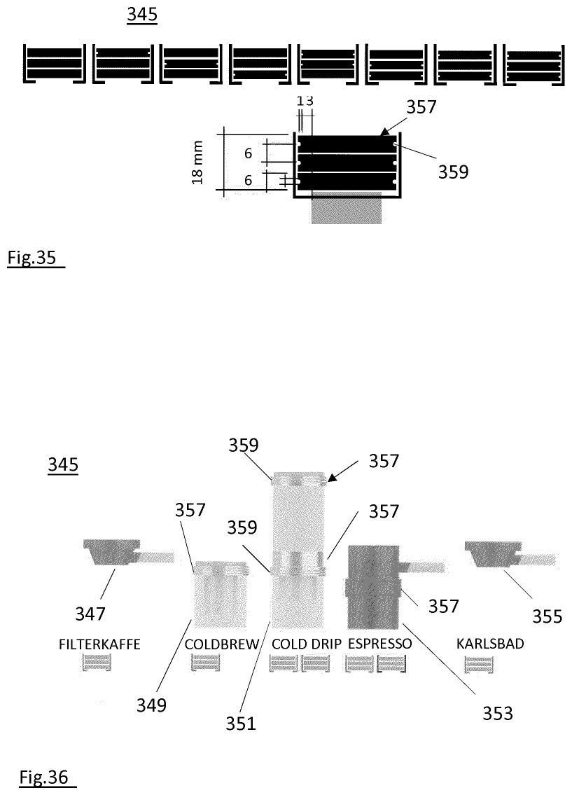

FIG. 35 shows a plurality of preparation devices for different ways of preparing coffee, and

FIG. 36 shows a plurality of preparation devices for different preparations of coffee.

As can be seen from FIGS. 1 to 26, the device features for preparation of infant and baby food, especially baby food and baby mash, will be first explained. However, the same device features are also suitable for preparing coffee, so that the device for preparing coffee is not described separately. It should be noted that the following description of the figures describes the device 1, 1′ according to the invention for dosing and/or preparing a medium to be prepared on the basis of the preparation of baby food, in particular baby milk or baby food, and coffee. However, it is conceivable that other media, for example tea, can be prepared accordingly.

First, with reference to FIGS. 1 to 9, an exemplary embodiment of a device 1 for preparing infant or baby food, in particular baby milk and baby mash, is explained.

As can be seen from FIGS. 1 to 3, the device 1 for the preparation of baby food (i.e. food that is particularly suitable for feeding infants), in particular baby milk and baby mash, comprises a housing 3 with a first receiving area 5 and a second receiving area 7.

The first receiving area 5 is designed to at least partially receive a first container 9 for baby food concentrate. The first receiving area 5 comprises a rear wall 13, two spaced apart side walls 15, 17, which are oriented at an angle different from 0° or 180°, in particular transversely to the rear wall 13, an upper limitation 19 and a lower limitation 21, which is below a angles different from 0° or 180°, in particular transversely to the side walls 15, 17. Furthermore, the first receiving area 5 comprises an at least partially open front side 23 opposite the rear wall 13, so that the first receiving area 5 is formed between the side walls 15, 17, the upper and lower limitation 19, 21, the rear wall 13 and the at least partially open front side 23. The upper and lower limitation 19, 21 can be arranged essentially parallel to each other, and the rear wall 31 can be arranged essentially transversely to the upper and lower limitation 19, 21 so that the upper and lower limitation 19, 21 each run in one plane which is perpendicular to the plane in which the rear wall is arranged.

In the description of the figures, terms such as above, top, left, right, front, back, horizontal, vertical, above, below, etc. relate to the exemplary representation of a device 1 for preparing baby food selected in the respective figures. In particular, the terms horizontal and vertical relate to the planes in which the upper limitation 19 and the lower limitation 21 of the device 1 extend.

The first receiving area 5 comprises an upper container receiving area 25 for receiving the first container 9 and a lower dosing device receiving area 27 for receiving a dosing device 29. The container receiving area 25 is preferably arranged above the dosing device receiving area 27.

Furthermore, FIGS. 1 to 3 show the second receiving area 7 in the device 1 for the preparation of baby food, which is designed to at least partially receive a second container 11 for a fluid (in particular liquid). The second receiving area 7 has a rear wall, two spaced apart side walls which are oriented at an angle different from 0° or 180°, in particular transversely to the rear wall, a lower limitation which is at an angle different from 0° or 180°, in particular transversely is aligned with the side walls, and an open top opposite the lower wall, the second receiving area 7 for receiving the second container 11 being formed between the side walls. The side walls of the second receiving area have one or more, preferably a plurality of clamping elements 155, which extend from a front side of the second receiving area 7 opposite the rear wall to the rear wall and are designed to position the second container 11 in the device 1 and/or to dose the fluid inside the second container and/or to store the fluid in a sterile manner inside the second container. The clamping elements 155 are designed in particular as clamps, two of the clamps being arranged opposite each other and in a plane parallel to the lower limitation. The distance of the clamping elements 155 relative to the lower limitation and/or relative to the open upper side can be changed. The lower limitation of the second receiving area 7 has a through hole which is designed to receive an outlet of the second container 11.

Furthermore, the device comprises a tempering device for tempering of the fluid (not shown in FIGS. 1 to 3). This tempering device can bring the temperature of the fluid (in particular the liquid) in the second container 11 to the preparation temperature (in particular provided or predetermined by the manufacturer of the baby food concentrate). The tempering device is preferably arranged at least partially in contact with the second container 11, preferably in an area adjoining or near to the lower limitation of the second receiving area 7.

Furthermore, the device for preparing baby food comprises a preparation device for preparing the baby food from the baby food concentrate and the fluid (not shown in FIGS. 1 to 3). Preferably, the preparation device is coupled to that of the device 1 so that baby food concentrate from the outlet opening of the screw conveyor housing in the first receiving area and/or fluid from the second container 11 in the second receiving area is poured into a separate container, preferably into a baby bottle.

With reference to FIGS. 4 to 14, the first receiving area 5, the first container 9, and the dosing device 29 are further described in more detail.

FIG. 4 shows that the dosing device receiving area 27 has an actuating and/or drive device 39 for the dosing device 29. The actuation and/or drive device 39 is arranged in or on the rear wall 13 and/or comprises a coupling element or drive shaft 41 which extends essentially away from the rear wall 13. The lower limitation 21 has a receptacle 43 for the dosing device 29, which extends essentially from the open front side 23 to the rear wall 13 essentially along a receptacle longitudinal axis 45. The drive shaft 41 in the rear wall 13 and the receptacle 43 for the dosing device 29 extend in the same plane transversely to the lower limitation 21 and/or essentially perpendicular to the receptacle longitudinal axis 45.

The receptacle 43 has in particular a cross-section transverse to the receptacle longitudinal axis 45 which is concave. In other words, the receptacle 43 is embedded in the lower limitation 21 as a concave section. The lower limitation 21 can thus have a surface which has a first horizontal surface section 47 adjoining or near to a first of the side walls 15, 17 and a second horizontal surface section 49 adjoining or near to the second of the side walls 15, 17, the receptacle 43 is arranged as a concave surface section between the first and second surface sections 47, 49. A receptacle outlet opening 51 can be arranged on the receptacle longitudinal axis 45, in particular adjoining or near to the rear wall 13.

A first guide element 31 and a second guide element 33 are arranged between the container receiving area 25 and the dosing device receiving area 27, the guide elements 31, 33 extending from the open front 23 to the rear wall 13 and/or wherein the guide elements 31, 33 extend from the side walls 15, 17 extend away. The guide elements 31, 32 divide the first receiving area 5 into the upper container receiving area 25 and the lower dosing device receiving area 27 so that the upper container receiving area 25 is limited by the upper limitation 19 and by the two guide elements 31, 33 on opposite sides. The lower dosing device receiving area 27 is limit by the two guide elements 31, 33 and the lower limitation 21 on opposite sides.

After the first container 9 and the dosing device 29 have been introduced into the first receiving area 5 and/or at least partially received, the first container 9 is arranged between the guide elements 31, 33 and the upper limitation 19 and the dosing device 29 is at least partially between the guide elements 31, 33 and the lower limitation 21 are arranged. The guide elements 31, 33 are aligned essentially in a plane parallel to the upper limitation 19 and to the lower limitation 21 and are inclined towards the open front side 23 out of the plane upwards towards the container receiving area 25. The guide elements 31, 33 thus have an essentially parallel area 35 and an inclined area 37. The essentially parallel area 35 extends from the rear wall 13 to the inclined area 37. The inclined 37 extends from the essentially parallel region 35 to the open front side 23.

One or more side walls 15, 17 in the container receiving area 25 comprise a plurality of ribs 53 which extend away from the one or more side walls 15, 17. The plurality of ribs 53 extends in particular essentially parallel to the upper limitation 19 and/or lower limitation 21. The plurality of ribs 53 preferably extend from the open front side 23 to the rear wall 13.

In particular, the plurality of ribs 53 is arranged in pairs on the two side walls 15, 17. Two of the ribs 53 each extend as pairs of ribs 55 in a plane essentially transversely to the side walls 15, 17 and/or essentially parallel to the upper or lower limitation 19, 21. The pairs of ribs 55 are regularly arranged at preferably equal intervals on the side walls 19, 21 in the container receiving area 25, preferably between the guide elements 31, 33 and the upper limitation 19.

The dosing device 29 is designed for dosing the baby food concentrate from the first container 9 from the second container 11. The dosing device 29 can be connectable to the first container 9. Thus, the dosing device 29 can be connected to the first container 9, so that the dosing device 29 in the state connected to the first container 9 can be introduced or received into the first receiving area 5 by a movement that is essentially perpendicular to the rear wall 13 of the first receiving area 5.

The dosing device 29 comprises a screw conveyor 57 and a screw conveyor housing 59, which are shown in FIGS. 5 to 7. As shown in FIG. 8, the screw conveyor 57, preferably in its full length, can be inserted into the screw conveyor housing 59 and rotatably arranged therein, so that the screw conveyor 57 and the screw conveyor housing 59 extend around a common screw conveyor longitudinal axis 61. The following describes the features of the screw conveyor 57 and the screw conveyor housing 59 in relation to the common screw conveyor longitudinal axis 61, even if the screw conveyor 57 is not inserted into the screw conveyor housing 59, as shown in FIGS. 5 to 7.

The screw conveyor housing 59 has an inlet 63 with an inlet opening 35 and/or an outlet 67 with an outlet opening 69. The inlet 63 and the outlet 67 are arranged in the screw conveyor housing 59 on opposite sides, seen transversely to the longitudinal axis 61 of the screw conveyor. The screw conveyor housing 59 extends between a first end 71 and an opposite second end 73 along the longitudinal axis 61 of the screw conveyor. The outlet 67 is disposed adjoining or near to the first end 71 and the inlet 63 is disposed adjoining or near to the second end 73.

The inlet 63 comprises in particular a flange 75 with a peripheral wall 77, the peripheral wall 77 at least partially surrounding the inlet opening 65 and/or extending essentially radially to the longitudinal axis 61 of the conveyor screw away from the screw conveyor housing 59. As FIG. 6 shows, the peripheral wall 77 extends essentially along a first peripheral wall central longitudinal axis 78. The peripheral wall center longitudinal axis 78 can in particular have a length of approximately 47 mm. Furthermore, the peripheral wall 77 extends along a second peripheral wall central longitudinal axis 80, which is oriented at an angle different from 0° or 180°, in particular transversely, preferably perpendicular to the first peripheral wall central longitudinal axis 78, and in particular can have a length of approximately 29 mm.

The flange 75 is designed for connecting the dosing device 29 to the first container 9 and/or for introducing the dosing device 29 into the dosing device receiving area 27. Thus, the peripheral wall 77 comprises a first contact surface 79 and a essentially opposite second contact surface 81, the first and second contact surfaces 79, 81 being aligned parallel to each other. These contact surfaces 79, 81 enable the dosing device 29 to be introduced into the dosing device receiving area 27 in a particularly simple manner. In particular, during insertion into the dosing device receiving area 27, the contact surfaces 79, 81 can slide essentially along the guide elements 31, 33 in the first receiving area 5 and/or, after being received in the dosing device receiving area 27, can essentially rest against the lateral guide elements 31, 33.

The screw conveyor housing 59 in particular has an outer wall 83 with a plurality of ribs or screw conveyor housing ribs 85, the ribs 85 preferably extending in the axial direction at least partially between the first end 71 and the second end 73 of the screw conveyor housing 59. The ribs 85 extend away from the outer wall 83 essentially in the radial direction as seen from the longitudinal axis 61 of the conveyor screw.

Two of the ribs 85, as the first pair of limiting ribs 87, limit the outlet opening 69 of the screw conveyor housing 59 on opposite sides in the peripheral direction of the outer wall 83. Two further ribs 85, as a second pair of limiting ribs 89, limit outlet opening 69 on opposite sides in the axial direction of outer wall 83. This configuration prevents baby food concentrate from coming into contact with the first receiving area 5, which, in contrast to the dosing device 29 and the container 9, cannot be exchanged.

The conveyor screw 57 has a drive end 82 in the direction of the conveyor screw longitudinal axis 61. From or at the drive end 82 of the screw conveyor 57, a coupling device 91 extends essentially along the longitudinal axis 61 of the screw conveyor, the coupling device 91 being designed to interact, in particular to intervene, with the actuating and/or drive device 39 or with the drive shaft 41.

The coupling device 91 can be designed as an essentially cylindrical cavity 93 or as a receptacle, so that after the introduction and at least partial inclusion of the dosing device 29 in the dosing device receiving area 27, the coupling element 41 in the dosing device receiving area 27 is at least partially received in the (cylindrical) recess 93 can be. The inner wall 95 of the (cylindrical) cavity 93 preferably has an inner profile which can be brought into engagement with an outer profile of the outer wall 96 of the coupling element 41. The outer profile of the coupling element 41 has at least one material elevation 97, which can engage or interact with at least one material recess 99 in the inner profile of the cylindrical cavity 93. Thus, the coupling element 91 is designed as a drive shaft, so that the introduction of the coupling element 41 into the (cylindrical) cavity 93 enables the dosing device 29 to be driven and thus the screw conveyor 57 to rotate.

The screw conveyor 57 is preferably designed as a shaft around which one or more helically wound flights 101 in the form of flat guide surfaces or sheets or rubber flaps are wound, which extend in the form of a screw thread 107 transversely away from the longitudinal axis 61 of the conveyor screw.

FIG. 8 shows the dosing device 29 inserted and received by the dosing device receiving area 27 in the first receiving area 5 of the device 1 for preparing baby food. The screw conveyor 57 preferably extends inside the screw conveyor housing 59, the coupling element 41 on the rear wall 13 being inserted into the cylindrical cavity 93 of the screw conveyor 57 and/or being able to drive it. Baby food concentrate that is introduced into the interior of the screw conveyor housing 59 through the inlet opening 65 can be guided along the one or more helically wound flights 101 essentially along the longitudinal axis 61 of the conveyor screw to the outlet 67 and/or through the outlet opening 69 the interior of the screw conveyor housing 59 leave. An insertion element or removal element 105 is preferably arranged on the second end 73 of the screw conveyor housing 59.

FIG. 9 shows a front view of the dosing device receiving area 27 with the dosing device 29 inserted. In the state inserted into the dosing device receiving area 27 of the first receiving area 5, the lateral contact surfaces 79, 81 of the peripheral wall 77 of the dosing device 29 and two of the ribs 85 rest on the two guide elements 31, 33. In particular, the abutment surfaces 79, 81 can abut the edges 107 of the guide members 31, 33 extending away from the side walls 15, 17, and the two ribs can abut the undersides 109 of the two guide members 31, 33 facing the lower limitation 21.

With reference to FIGS. 10 to 14, exemplary embodiments of the first container 9 for receiving and/or for dosing baby food concentrate are described.

As can be seen from FIG. 10, the first container 9 can have a housing 111 having an interior space 112 for receiving baby food concentrate and an outlet 113 in fluid communication with interior space 112. The first container 9 is designed to be introduced and/or received at least partially in a device 1 for preparing baby food as previously mentioned in the context of FIGS. 1 to 9.

The outlet 113 can be connected to an inlet of a dosing device 29, which comprises a screw conveyor 57 and a screw conveyor housing 59, the screw conveyor 57, preferably in its full length, being inserted into the screw conveyor housing 59 and arranged to be rotatable, so that the screw conveyor 57 and the screw conveyor housing 59 extend around a common longitudinal axis, the conveyor screw longitudinal axis 61. The outlet 113 of the first container 9 can be connected to the inlet 63 of the screw conveyor housing 59, the screw conveyor housing 59 having an outlet 67, so that by actuating the dosing device 29, a predetermined or predeterminable amount (or dosage) of baby food concentrate through the outlet 67 is delivered through. The outlet 113 of the first container 9 can be screwed or glued to the inlet 63 in the screw conveyor housing 59, or the inlet 63. The screw conveyor housing 59 can, however, also be integrated into the first container 9 or firmly connected to it.

The first container 9 can have an inlet opening 115, the inlet opening 115 preferably being arranged essentially opposite the outlet 113 and/or an outlet opening 117 in the outlet 113. The inlet opening 115 can be closable by means of a closure element 119, preferably by means of a zipper or zip. The closure element 119, preferably the zipper, is designed to be inserted at least partially into a groove in the first receiving area 5 of the device 1 for preparing baby food. A tab 121 with an inner opening 123 is arranged adjoining or near to the closure element 119. The inner opening 123 can serve as a handle, so that the first container can be easily carried from one location to another.

As can be seen from FIGS. 11 and 12, the first container 9 at least partially has a tapering section 125, the periphery of the first container 9 decreasing in the tapering section 125 towards the outlet 113 (preferably essentially conical) or tapered.

The first container 9 can have a cross-section transverse to the longitudinal axis 61 of the screw conveyor housing 59 (seen in the state connected to the first container 9), the tapering section 125 being laterally limited by a first side edge 135 and a second side edge 137. The first side edge 135 can essentially be at an angle different from 0° or 180°, in particular essentially transversely, preferably at an angle less than 90°, particularly preferably at an angle of approximately 45°, to the plane of the conveyor screw longitudinal axis 61 of the screw conveyor housing 59 (as seen in the connected state). The second side edge 137 can run at an angle different from 0° or 180°, in particular essentially transversely, preferably at an angle less than 90°, particularly preferably at an angle of approximately 45°, to the plane of the conveyor screw longitudinal axis 61 of the screw conveyor housing 59. The first side edge 135 and/or the second side edge 137 can each have a side edge section 136 which can extend at an angle of 90° to the plane of the conveyor screw longitudinal axis 61 of the screw conveyor housing 59 (seen in the connected state). The side edge portion 136 may extend in the plane of the first side edge 131 or the second side edge 133 of the essentially symmetrical portion.

The first container 9 can at least partially have a first essentially symmetrical section 127. The periphery of the first container 29 within the first essentially symmetrical section 127 is preferably constant. The first essentially symmetrical section 127 is spaced further from the outlet 113 than the tapered section 125. The first container 9 can have a cross-section transverse to the longitudinal axis 61 of the screw conveyor housing 59 in the state connected to the first container 9, the first essentially symmetrical section 127 being laterally limited by a first side edge 131 and a second side edge 133. The first side edge 131 and the second side edge 133 are oriented essentially parallel to each other and/or at an angle different from 0° or 180°, in particular essentially transversely, preferably at an angle of approximately 90°, to the plane of the longitudinal axis 61 of the conveyor screw of the screw conveyor housing 59 (seen in the connected state). The first side edge 131 of the first essentially symmetrical section 127 can run in one plane with the first side edge 135 of the tapered section 125 and/or the second side edge 133 of the first essentially symmetrical section 127 can be oriented at an angle different from 0° or 180, in particular be oriented essentially transversely to the second lateral edge 137 of the tapering portion 125.

Adjoining or near to the outlet 113, the first container 9 can have a second essentially symmetrical section 129, the circumference of the first container 9 within the second essentially symmetrical section 129 preferably remaining essentially the same and essentially the periphery of the outlet 113 and/or corresponds to the periphery of an outlet opening in outlet 113. The second essentially symmetrical section 129 can serve as an outlet through which the baby food concentrate can be guided out of the interior space 112 of the first container 9. The side edges 139 and 141 of the second essentially symmetrical section 129 can preferably have a length of 10 to 30 mm, particularly preferably a length of 15 mm.

However, it is also conceivable that the side edges 139 and 141 of the second essentially symmetrical section 129 have a length of greater than 30 mm, preferably between 70 and 110 mm, particularly preferably 90 mm. This can be provided in particular if the dosing does not take place by means of the dosing device 29 with a screw conveyor 58 and a screw conveyor housing 59, as previously described, but if clamping elements or clamps are used to dose the baby food concentrate adjoining or near to the side walls 15, 17 in the first receiving area 5, which are designed analogously to the clamping elements 155 or brackets, which are described later in connection with the second container 11 and the second receiving area 7.

The first container 9 can have a cross-section transversely to the longitudinal axis 61 of the screw conveyor housing 59 in the state connected to the first container 9, the second essentially symmetrical section 129 being laterally limited by a first side edge 139 and a second side edge 141, which in the Is aligned essentially parallel to each other. The first side edge 139 and the second side edge 141 run at an angle different from 0° or 180°, in particular essentially transversely, preferably at an angle of approximately 90°, to the plane of the conveyor screw's longitudinal axis 61 of the screw conveyor housing 59 (seen in the connected state). The first side edge 139 of the second essentially symmetrical section 129 can run in one plane with the first side edge 135 of the tapering section 125 and with the first side edge 131 of the first essentially symmetrical section 127.

The tapered portion 125 is disposed between the first essentially symmetrical portion 127 and the second essentially symmetrical portion 129. The second essentially symmetrical section 129 preferably has a circumference that is smaller than the periphery of the first essentially symmetrical section 127. The second essentially symmetrical section 129 surrounds a volume of the first container 29 which is smaller than a volume which surrounds the first essentially symmetrical section 127.

FIG. 13 shows further, conceivable embodiments of the first container 9, which can be connected to the dosing device 29 and/or which is designed to be inserted and received in the device 1 as previously described for preparing baby food. The dosing device 29 can comprise a plate 143 which is designed as a stand plate and can be arranged on the screw conveyor housing 59 or can be connected to the screw conveyor housing 59. This stand plate 143 is used in particular to position or align the first container 9 better and/or to protect it from falling over, in particular when the first container 9 is positioned to receive baby food concentrate. It is also conceivable that the screw conveyor housing 59 has a cover 145, the cover 145 having at least one flat surface 147 which serves as a base and/or a base so that the first container 9 can be better positioned and is protected from falling over. However, it is also conceivable that the first container 9 has a further, essentially symmetrical section instead of the tapering section 125. The first side edges 131, 135, 139 of the three sections can run in one plane and the second side edges 133, 137, 141 can run in one plane, the two planes being oriented essentially parallel to each other.

FIG. 14 shows a further embodiment of the first container 9, in which one and the same opening 117 is used to receive the baby food concentrate in the first container 9 and to remove the baby food concentrate from the first container 9. In this case the opening corresponds to the outlet opening 117, so that the first container 9 does not have a separately designed inlet or inlet opening. The baby food concentrate is first received through the outlet 113 or the outlet opening 117 into the first container 9, and the outlet 113 is then connected to the dosing device 29. In particular, after the baby food concentrate has been taken up, the outlet 117 can be connected to the inlet 63 of the dosing device 29 by means of a connecting element 149, for example an adhesive element in the form of an adhesive strip 151 or a clip 153.

With reference to FIGS. 15A to 24, the second receiving area 7 of the device 1, the second container 9, and the dosing device 29 will be described in more detail.

As FIG. 15A shows, the second receiving area 7 has a rear wall 157, two spaced-apart side walls 159 which are oriented transversely to the rear wall 157, a lower limitation 161 which is oriented transversely to the side walls 159, and one opposite to the lower limitation 161 open top 163. The second receiving area 7 for receiving the second container 11 is formed between the side walls 159. Adjoining or near to the side walls 159 are arranged a multiplicity of clamping elements 155 which extend at least partially between a front side 159 opposite the rear wall 157 and the rear wall 165 of the second receiving area 7.

The embodiment of the second receiving area 7 illustrated in FIGS. 15A to 15C comprises three pairs of clamping elements 167, 169, 171 which are arranged adjoining or near to the side walls 159. Each of the clamping element pairs 167, 169, 171 is arranged in a plane which is arranged essentially parallel to the lower limitation 161 of the second receiving area 7. The clamping elements 155 can be designed as clamps, two of the clamps being arranged opposite each other and in the plane parallel to the lower limitation 161 of the second receiving area 7. It is conceivable that one of the clamping elements of the first, lower clamping element pair 167 is replaced by the tempering device (not shown). Thus, the second container 11 can be closed or clamped by the interaction of a clamping element with the tempering device, the temperature of the fluid inside the second container 11 being able to be controlled at the same time.

A first of the clamping element pairs 167, 169, 171 is arranged as a lower clamping element pair 167 in such a way that the clamping elements of the first, lower clamping element pair 167 have a first distance D1 from the lower limitation 161. A second of the clamping element pairs 167, 169, 171 is arranged as the upper clamping element pair 169 in such a way that the clamping elements of the second, upper clamping element pair 169 have a second distance D2 to the lower limitation 161, which is greater than the first distance D1 to the lower limitation 161. The first, lower pair of clamping elements 167 can be arranged adjoining or near to the lower limitation 161 in the second receiving area 7. The second, upper pair of clamping elements 169 is arranged adjoining or near to the open upper side 163. Furthermore, a third of the clamping element pairs 167, 169, 171 can be arranged as a middle clamping element pair 171 between the first clamping element pair 167 and the second clamping element pair 169 and have a third distance D3 to the lower limitation 161, which is greater than the first distance D1 and less than the second distance D2.

The distance or distance of the clamping elements 155 relative to the lower limitation 161 or relative to the open upper side 163 can be changed. In particular, the third, middle pair of clamping elements 171 is height-adjustable in that the third distance D3 can be changed. This enables the exact dosage of the desired amount of fluid (in particular amount of liquid) for the preparation of baby food. The first, lower clamping element pair 167 and the second, upper clamping element pair 169 can, however, also be adjustable in height, so that the first distance D1 and the second distance D2 can be changed.

The second container 11 can, as shown in FIGS. 15A and 15B, be inserted into the second receiving area 7 and, as shown in FIG. 15C, can be received by the second receiving area 7. 15C shows the state of the second container 11 received in the second receiving region 7. The second container 11 is introduced into the second receiving area 7 by a movement that is essentially perpendicular to the lower limitation 161.

In the state received in the second receiving area 7, the second container 11 is held or clamped laterally by three pairs of clamping elements 167, 169, 171. The three pairs of clamping elements 167, 169, 171 can assume a first position (see FIG. 15C) and a second position (not shown). In the first position, the three pairs of clamping elements 167, 169, 171 laterally adjoin the second container 11 and/or touch the second container 11 in such a way that the pairs of clamping elements 167, 169, 171 exert pressure on opposing first and second side walls 173, 175 of the second container 11 exercise. In the second position, the three pairs of clamping elements 167, 169, 171 do not adjoin the second container 11 or they do not touch the second container 11, so that the pairs of clamping elements 167, 169, 171 do not exert any pressure on the side walls 173, 175 of the second container 11.

FIG. 16 shows an embodiment of one of the three pairs of clamping elements 167, 169, 171 and the arrangement of a first clamping element 177 and a second clamping element 179 of one of the clamping element pairs 167, 169, 171 relative to each other. In the position in which the first clamping element 177 and the second clamping element 179 are arranged relative to each other, the individual clamping elements of the three clamping element pairs 167, 169, 171 in the second receiving region 7 are also arranged relative to each other.

Each of the clamping elements 155,177,179 comprises a first clamping element surface 181 and a second clamping element surface 183, the clamping element surfaces 181, 183 being arranged on opposite sides of a clamping element longitudinal axis 185, the two clamping element surfaces 181, 183 preferably being connected by means of a third clamping element surface 186, and the third clamping element surface 186 has a essentially conically shaped cross section transverse to the clamping element longitudinal axis 185.

The first and second clamping element surfaces 181, 183 are arranged essentially parallel to each other and each extend in the direction of the clamping element longitudinal axis 185 between a first end 187 and a second end 189. The first clamping element surface 181 extends in a first plane and the second clamping element surface 183 extends in a second plane, wherein the first plane and the second plane are aligned parallel to each other and/or wherein the clamping element longitudinal axis 185 is in a plane between the first and second level is arranged. The width B of the two clamping element surfaces 181, 183, i.e. the width B of the two clamping element surfaces 181, 183 at an angle different from 0° or 180°, in particular transverse to the clamping element longitudinal axis 185, tapers from the first end 187 to the second end 189. Furthermore, each of the clamping elements 177, 179 comprises a connecting plate 191 which is arranged at an angle different from 0° or 180°, in particular transversely to the clamping element longitudinal axis 185. The first clamping element surface 181 is connected with the first end 187 to the connection plate 191 and the second clamping element surface 183 can be connected with the first end 187 to the connection plate 191.

With the connecting plate 191, the individual clamping elements 155, 177, 179 can be connected to the second receiving area 7, in particular to the rear wall 157 of the second receiving area 7, so that the clamping elements 155, 179, 179 are essentially transverse to the rear wall 157 in one of 0° or 180°, in particular transversely, so that the second end 189 of the individual clamping element surfaces 181, 183 are spaced from the rear wall 157 and the clamping elements 155, 177, 179 are adjoining or near to the side walls 173, 175 between the front side 165 and the rear wall 157 extend.

The third clamping element surface 186 extends from a first side edge 193 of the first clamping element surface 181 to a first side edge 195 of the second clamping element surface 183. The first side edges 193, 195 extend in the same plane, which extends at an angle different from 0° or 180°, preferably at an angle of 90°, in particular transversely to the longitudinal axis 185 of the clamping element. The third clamping element surface 186 is arranged at an angle of 90° to the first clamping element surface 181 and to the second clamping element surface 183 and in each case at an angle of 90° to the connecting plate 191 and to the rear wall 157 of the second receiving area 7 when the connecting plate 191 is connected to the rear wall 157 is. Each of the clamping elements 155, 177, 179 of the individual clamping element pairs 167, 169, 171 can thus have a third clamping element surface 186, the third clamping element surfaces 181 of the individual clamping elements 155, 177, 179 of each clamping element pair 167, 169, 171 being aligned parallel to each other when the clamping elements 155, 177, 179 are connected to the rear wall 157 by means of the connecting plate 191. The connecting plate 191 has at least one through hole 192, so that the clamping elements 155, 177, 179 can be connected to the rear wall 157 by means of a connecting element, for example a screw. However, it is also conceivable that the clamping elements 155, 177, 179 are not connected to the rear wall 157 by means of a connecting plate 191, but that the clamping elements 155, 177, 179 are displaceable on the rear wall by means of a slide or by means of a rail or guide rail element 157 and/or are arranged or connectable on the side walls 159 of the second receiving area 7.

The third clamping element surface 186 can have a clamping element edge 197 which extends essentially in the direction of the clamping element longitudinal axis 185 and between the first side edge 193 of the first clamping element surface 181 and the first side edge 195 of the second clamping element surface 183. The clamping element edge 197 extends in the same plane as the clamping element longitudinal axis 185.

The clamping elements 155, 177, 179 are designed to be open opposite the third clamping element surface 186 and viewed transversely to the clamping element longitudinal axis 185. In other words, each of the clamping elements 155, 177, 179 comprises an inner cavity 199 which is limited by the three clamping element surfaces 181, 183, 186 and has an open side 201. However, it is also conceivable that the clamping elements 155, 177, 179 are designed without an inner cavity 199. When the clamping elements 155, 177, 179 are connected to the second receiving area 7, for example when the clamping elements 155, 177, 179 are connected to the rear wall 157 by means of the connecting plate 191 or when the clamping elements 155, 177, 179 are connected by means of a slide or rail or guide rail element are connected to the rear wall 157 and/or the side walls 159, the open side 201 of the inner cavity 199 points to one of the two side walls 159 of the second receiving area 7. At least one of the clamping element surfaces 181, 183, 186, preferably the third clamping element surface 186, can be designed as a support surface, preferably a rubberized support surface. Preferably, each of the clamping elements 155, 177, 179 comprises a spring element (not shown). The spring element can be arranged adjoining or near to the first end 187 of the first clamping element surface 181 or the second clamping element surface 183. The clamping elements 155, 177, 179 can be formed from an elastomer or comprise an elastomer in order to generate a uniform surface pressure when the second container 11 is held or clamped by the clamping elements 155, 177, 179.

The tempering device can be arranged in contact with the second container 11, the tempering device preferably being arranged in an area adjoining or near to the lower limitation 161 of the second receiving area 7 and/or wherein the tempering device is arranged in an area adjoining or near to one of the clamping elements 155, 177, 179 which is closest to the lower limitation 161 is arranged. The tempering device is therefore preferably arranged or in contact at the lowest point of the second container 11 (in the state inserted into the second receiving area 7). This ensures uniform heating and/or mixing of the fluid inside the second container 11,

The tempering device can be arranged adjoined to or near to the first, lower pair of clamping elements 167. When the second container 11 is received by the second receiving area 7, a lower area of the second container 11 is arranged adjoining, preferably adjacent, to the tempering device so that the fluid (especially the liquid) inside the second container 11 can be temperature controlled.

FIGS. 17A and B show a possible embodiment of the second container 11 for receiving and dosing fluid for the preparation of baby food, which is designed to be introduced and received in a device 1 for the preparation of baby food. The second container 11 comprises a housing 203 with an interior space 205 for receiving fluid, an inlet 207 in fluid connection with the interior space 205 and an outlet 209 in fluid connection with the interior space 205. The housing 203 can be formed from a flexible material, for example from a film material. Furthermore, the inlet 207 of the second container 11 can be connected to an outlet 211 or to an outlet opening 255 of a fluid reservoir 213, such that a dosing of the fluid for the preparation of baby food can be delivered through the outlet 209 of the second container 11.

The inlet 207 of the second container 11 comprises an inlet opening 215, which is preferably essentially opposite to the outlet 209 of the second container 11 in the direction of a container longitudinal axis 217 and/or essentially opposite to an outlet opening 219 of the outlet 209 of the second container 11 in the direction of container longitudinal axis 217 is arranged seen. The second container 11 thus comprises an inlet 207 with an inlet opening 215 and an outlet 209 with an outlet opening 219, the outlet 209 being arranged on a side opposite to the inlet 207.

When the second container 11 is introduced into the second receiving area 7 by an essentially vertical movement through the open top 163, the second container 11 is received by the second receiving area 7 such that the outlet 209 is in a lower area 221 of the second receiving area 7 is arranged, adjoining or near to the first, lower clamping element pair 167 and adjoining or near to the lower limitation 161. For example, the outlet 209 of the second container 11 can be passed through the through-hole 223 in the lower limitation 161 of the second receiving area 7, preferably by a essentially perpendicular movement to the lower limitation 161 (see FIG. 18). At the same time, the inlet 207 of the second container 11 is arranged in an upper area 225 of the second receiving area 7, adjoining or near to the open top 163 and adjoining or near to the second, upper pair of clamping elements 169.

The outlet 209 of the container 11 is designed for insertion into the through-hole 223 in the lower limitation 161 of the second receiving area 7.

The outlet 209 of the second container 11 can be configured as an elongated element, for example the outlet 209 can be tubular and extend between a first end 227 and an opposite second end 229 along a longitudinal outlet axis 231. The outlet 209 comprises one outside diameter that is smaller than the inside diameter of through-hole 223 in the lower limitation 161 of the second recording area 7. Hence, the outlet 209 of the second container 11 can through the through-hole 223 in the lower limitation 161 of the second receiving area 7 are passed through when the second container 11 is inserted into the second receiving area 7. The second end 229 of the outlet 209, when the second container 11 is received in the second receiving area 7, can lie in a plane which is below the plane of the lower limitation 161.

The second container 11 extends between an inlet end 233 and an opposite outlet end 235 along the longitudinal axis 217 of the container. Adjoining or near to the inlet end 233, the inlet opening 215 and the inlet 207 are arranged. The outlet 209 and the outlet opening 219 are arranged adjoined or near to the outlet end 235. The outlet longitudinal axis 231 can run in the same plane as the container longitudinal axis 217, which essentially corresponds to a central longitudinal axis of the second container 11. However, it is also conceivable that the outlet longitudinal axis 231 runs in a plane parallel to the container longitudinal axis 217.

The second container 11 has a first side wall 173 and an opposing second side wall 175 which extend essentially parallel to the plane of the container longitudinal axis 217 between the inlet end 233 and the outlet end 235. The second container 11 has at least partially a tapering section 241 in the lower region near to the outlet end 235, the periphery of the second container 11 decreasing, preferably essentially conically, in the tapering section 241 towards the outlet 209. In the tapering section 241, the distance between the first side wall 173 and the second side wall 175 decreases towards the outlet 209, preferably essentially conically. This enables the fluid (in particular the liquid) to be guided almost completely out of the second container 11 through the outlet 209.

The second container 11 has at least partially a essentially symmetrical section 243, wherein the periphery of the second container 11 remains the same within the essentially symmetrical section 243 and wherein the essentially symmetrical section 243 is further spaced from the outlet 209 of the second container 209 than the tapered portion 241.

In the essentially symmetrical section 243, the first side wall 173 and the second side wall 175 each extend in a plane parallel to the plane of the longitudinal axis 217 of the container. The essentially symmetrical section 243 extends between the inlet end 233 and the tapered section 241. When the second container 11 is inserted or received in the second receiving area, the clamping elements 155 of the individual clamping element pairs 167, 169, 171 adjoin the side walls 173, 175 in the essentially symmetrical section 243 and exert pressure on the side walls 173, 175.

Preferably, the distance between the two side walls 173, 175 in the essentially symmetrical section 243 is between approximately 20 mm to 60 mm (for example approximately 30 mm) and/or the length of the two side walls 173, 175 175 in the essentially symmetrical section 243 is in the range of approximately 150 mm to 300 mm (e.g. approximately 220 mm).

The distance between the two side walls 173, 175 decreases in the tapering section 241 from approximately 20 mm to 60 mm (e.g. approximately 30 mm) towards the second end 229 of the outlet 209 to approximately 10 mm to 50 mm (e.g. approximately 20 mm). Preferably, the distance between the two side walls 173, 175 in the outlet (209) is approximately 10 mm to 50 mm (e.g. approximately 20 mm).

As FIGS. 19 and 20 show, the inlet 207 of the second container 11 can be connected to an outlet 227 of the fluid reservoir 213, so that fluid (in particular liquid) can be dosed from the fluid reservoir 213 into the interior of the second container 111 and by means of the clamping elements 155 of individual pairs of clamping elements 167, 169, 171, the fluid can be dosed in the desired amount that is necessary for the preparation of baby food and guided out of the second container 11 through the outlet 209. The inlet 207 of the second container 11 can be firmly connected to the outlet 255 of the fluid reservoir 213, for example screwed or glued. The fluid reservoir 213 can, however, also be integrated into the second container 11.

The second container 11 can have a essentially horizontal plate 245 which is arranged adjoining or near to the inlet opening 215 and/or adjoining or near to the inlet 207 of the second container 11. The plate 245 is particularly advantageous when the fluid reservoir 213 is not designed as a dimensionally stable container or when it is not a combination container 284, as described later with reference to FIGS. 25A, 25B and 26. The plate 245 can preferably be connected to the second container 11 or the plate 245 is firmly connected to the second container 11 or the plate 245 is integrated into the second container 11. The plate 245 or suspension tab can be connected to the upper region near to the inlet end 233 of the second container 11 in a fixed or detachable manner. The plate 245 can be formed integrally with the second container 11. The plate 245 preferably has a surface shape that essentially corresponds to the surface shape of a cross section of the second container 11 at an angle different from 0° or 180°, preferably at an angle of 90°, in particular seen transversely to the longitudinal axis 217 of the second container 11, corresponds. The surface shape of the plate 245 is for example rectangular. The distance between two opposite sides of the plate is preferably equal to or greater than the distance between two opposite side surfaces, for example the distance between the first side wall 173 and the second side wall 175 of the second container 11 when the second container 11 is inserted into the second receiving area 7 and is assumed or if it is filled with a fluid or if a fluid is received in the interior space 205 of the second container 11. As FIGS. 19 and 20 show, when the second container 11 is accommodated in the second receiving area 7, the plate 245 rests on the edges or peripheral surfaces of the open top 163, so that the plate 245 at least partially, preferably completely, the open top 163 covers.

The plate 245 comprises a through hole 247, the through-hole 247 preferably comprising a first flange 249 with a first peripheral wall 251, the first peripheral wall 251 at least partially surrounding the through hole 247 and at an angle different from 0° or 180°, in particular extending essentially transversely from a first side 253 of the plate 245. The first flange 249 is designed to connect the plate 245 to the outlet 211 of the fluid reservoir 213 and/or to an outlet opening 255 of the fluid reservoir 213.

FIGS. 19 and 20 show an embodiment in which the first flange 249 can be connected to the outlet 211 of the fluid reservoir 213, for example by a plug connection. The fluid reservoir 213 is designed as a tank. Accordingly, the outer diameter of the first flange 249 or the first peripheral wall 251 is slightly smaller than the inner diameter of the outlet 211 of the fluid reservoir 213. Thus, the outlet 211 of the fluid reservoir 213 can be plugged onto the first flange 249 in a simple manner and thus connected to the first flange 249, so that the fluid can be reliably introduced into the second container 11.

FIG. 21 shows an exemplary embodiment in which the first flange 249 can be connected to the outlet 211 of the fluid reservoir 213 by a screw connection. The fluid reservoir 213 is a bottle in which fluid suitable for the preparation of baby food is received and which can be purchased, for example, in the supermarket. The first peripheral wall 251 of the first flange 249 comprises a first thread 257, the first thread 257 being arranged on the inside of the first peripheral wall 251, which faces the through-hole 247 in the plate 245. The first thread 257 is designed to be screwed to a second thread 259 on the outside of a peripheral wall 261 of the outlet 211 of the fluid reservoir 213.

The plate 245 can be connectable to the second container 11 or can be firmly connected to the second container 11 or integrated into the second container 11. In particular, the plate 245 can be firmly connected or integrated with the second container 11 with a second side 262, which is arranged opposite to the first side 253, the through-hole 247 of the plate 245 being fluidically connected to the inlet 207 of the second container 11.

As shown in FIG. 21 shows, the through-hole 247 can have a second flange 263 with a second peripheral wall 265, the second peripheral wall 265 at least partially surrounding the through-bore 247 and extending essentially transversely away from the second side 262 of the plate 245. The second flange 263 is designed to connect the plate 245 to the inlet 207 and/or to the inlet opening 215 of the container 11.

The second flange 263 and the second peripheral wall 265 are arranged on the second side 262 of the plate 245 such that the second flange 263 and the second peripheral wall 265 at least partially surround the through-hole 247 of the plate 245. The second flange 263 and the second peripheral wall 265 are configured essentially like the first flange 249 and the first peripheral wall 251. The first flange 249 and the second flange 263 surround the same flange center longitudinal axis 267, which extends at an angle different from 0° or 180°, in particular transversely to the plane of the plate 245 and/or through the through opening 247 of the plate 245. Thus, the plate 245 can be connected to the fluid reservoir 213 by means of the first flange 249 and to the inlet 207 of the second container 11 by means of the second flange 263, a fluid reservoir center longitudinal axis 269 of the fluid reservoir 213 and the container longitudinal axis 217 of the second container 11 are in the same straight line with the flange central longitudinal axis 267 in fig when the fluid reservoir 213, the plate 245, and the second container 11 are connected to each other.

FIG. 22 shows an embodiment in which the inlet 207 of the second container 11 can be connected directly to the outlet 211 of the fluid reservoir 213. The fluid reservoir 213 is designed in such a way that a plate 245 can be dispensed with. The outlet 211 of the fluid reservoir 213 can be connected to the inlet 207 of the second container 11 by means of a plug connection or by means of a screw connection as previously described; in the exemplary embodiment in FIG. 22, the inlet 207 of the second container 11 has the first thread 257 on the inside of the inlet 207, which points towards the inlet opening 215, and can be screwed to the second thread 259 on the outside of the outlet 211 relative to the outlet opening 255 of the fluid reservoir 213. The fluid reservoir 213 comprises a housing 271 with an upper side 273 and a lower side 275, the upper side 273 and the lower side 275 being arranged at opposite ends to the fluid reservoir central longitudinal axis 269. The outlet 211 of the fluid reservoir 213 comprises on the underside 275, the underside 275 running in a essentially horizontal plane or in a plane at an angle different from 0° or 180°, in particular at an angle of 90°, transversely to Fluid reservoir central longitudinal axis 269 seen. The design of the lower side 275 assumes the function of the plate 245.

As FIGS. 23A and 23B show, the second container 11 comprises at least one first magnet 277, wherein the at least one first magnet 277 is preferably arranged on an outer wall 279 of the tapering section 241 of the second container 11, and wherein the at least one first magnet 277 can be connected to at least one second magnet 281 adjoining or near to the through-hole 223 in the lower limitation 161 of the second receiving area 7. The at least one second magnet 281 is arranged on an inner wall 283 of the through-hole 223. The at least one first magnet 277 may include a first magnetic plate and a second magnetic plate, which on opposite sides are arranged at an angle different from 0° or 180°, in particular transversely to the longitudinal axis 217 of the container. The at least one second magnet 281 can have a first magnetic contact and a second magnetic contact, which are arranged on opposite sides of the through hole 223 on its inner wall. As FIG. 24 shows, the second container 11 can be correctly received or placed in the second receiving area 7 by the magnets 277, 281, so that the fluid for preparing baby food can be guided out of the outlet 209 of the second container 11 without fluid reaches the housing of the second receiving area 7. As an alternative to the first magnet 277 or the second magnet 281, a metal element (or a metal plate or a metal strip) can also be provided.

FIGS. 25A and 25B show a combination container 284 in which the second container 11 and the fluid reservoir 213 are connected to each other. The second container 11 and the fluid reservoir 213 can be connected to each other in one piece as a unit, the fluid reservoir 213 and/or the second container 11 being filled with fluid. It is also conceivable here that only the fluid reservoir 213 is filled with fluid and the second container 11 or the dosing and sterilization area is fastened to the fluid reservoir 213 in a folded-in manner. The fluid reservoir filled with fluid, for example a Tetra-Pak, can be separated from the second container 11 or from the dosing and sterilization bag with a clip or with an adhesive strip

However, it is also conceivable that the second container 11 and the fluid reservoir 213 are first produced separately from each other and then connected to each other as previously described, for example by gluing, and are filled with fluid. The combination container 284 can also comprise a positioning and holding device 285, which are provided as an alternative to the connecting plate 191 described above and which serve a essentially similar purpose. The positioning and holding device 285 is preferably designed as a clamp or as a C-clamp or as a C-holding element with a C-shape. This C-clamp is arranged between the second container 11 and the fluid reservoir 213, preferably at the point at which the second container 11 is connected to the fluid reservoir 213. The C-clamp or the C-holding element can for example be attached, preferably glued, to the underside of the fluid reservoir 213 or to the upper side of the second container 11.

As FIG. 26 shows, the combination container 284 can be positioned and held by means of the positioning and holding device 285 on one of the side walls 159 of the second receiving area 7, preferably in an upper area of the second receiving area 7. However, it is also conceivable that a cover with an opening is provided (not shown), by means of which the open top 163 of the second receiving area 7 can be closed or covered so that the combination container 284 is positioned on the cover by means of the positioning and holding device 285 and can be held. The positioning and holding device 285 can be arranged around the opening on the cover in such a way that the positioning and holding device 285 surrounds the opening and the second bag 11 is arranged below the cover and the fluid reservoir 213 is arranged above the cover when the combination container 284 is inserted into the second receiving area 7. The positioning and holding device 285 prevents the combination container 284, in the state inserted into the second receiving area 7, from sliding downwards in the direction of the lower limitation 161 while the fluid is being emptied. This ensures that the second container 11 can be completely emptied.

The system, as previously described, comprises a device 1 for preparing baby food, in particular baby milk or baby mash, a first container 9 for receiving and dosing baby food concentrate and a second container 11 for receiving and dosing fluid (in particular liquid), so that by means of this system the baby food can be prepared. For this purpose, the first container 9 is designed for receiving and dosing baby food concentrate and for receiving and interacting with the device 1 for preparing baby food. The second container 11 is designed for receiving and dosing fluid and for receiving and interacting with the device 1. The device 1 comprises the first receiving area 5, which is designed to receive the first container 9 and to receive and drive the dosing device 29 that can be connected to the container 9. Since the dosing device 29 is driven by the actuation and/or drive device 39, which is arranged in the dosing device receiving area 27 within the first receiving area 5, the baby food concentrate and/or the fluid can be correctly dosed. The device 1 also comprises the second receiving area 7, which is designed to receive the second container 11. The fluid can be correctly dosed by means of the clamping elements 155 which are arranged in the second receiving area 7. With the device 1, the baby food concentrate from the first container 9 and the fluid from the second container 11 can be conveyed to a preparation device and introduced into a container, in particular a baby bottle, in the correct mixing ratio. The device 1 thus enables a simplified, correct and safe preparation of baby food.

An exemplary embodiment of a device 1′ for dosing and grinding coffee powder and/or for preparing coffee is described with reference to FIGS. 27 to 29. In the following, the device 1′ for dosing and grinding coffee powder and/or for preparing coffee is explained solely on the basis of the features that differ from the device 1 as previously described. It is therefore conceivable that the device 1′ described below can also be used for the dosing and preparation of baby food if a first container 9 with baby food concentrate is inserted into the first receiving area 5′ of the device 1′ instead of the first container 9′ with coffee beans is used.

The device 1′ for dosing and grinding coffee powder and/or for preparing coffee differs from the device 1 previously described in FIGS. 1 to 26 in that the dosing device 29 is used as a dosing and grinding device 29′ for dosing and grinding coffee beans is configured, wherein the first receiving area 5′ has a dosing and grinding device receiving area 27′ for receiving the dosing and grinding device 29′, and in the dosing and grinding device receiving area 27′ an actuating and/or drive device 39 for the dosing and Grinding device 29′ is arranged.

FIG. 27 shows the device 1′, a first container 9′ for coffee beans and the dosing and grinding device 29′ connected to it being inserted into the first receiving area 5′ of the device 1′. Furthermore, the second container 11 is inserted into the second receiving area 1′ of the device 1′.

FIGS. 28 and 29 show the dosing and grinding device 29′. The dosing and grinding device 29′ comprise a screw conveyor 57′, a grinder 287 and a screw conveyor housing 59. The grinder 287 is preferably inserted in its full length into the screw conveyor housing 59 and is rotatably arranged therein and the screw conveyor 57′ is preferably inserted in its full length into the screw conveyor housing 59 and is rotatably arranged therein. When inserted into the screw conveyor housing 59, the grinding mechanism 287 and the screw conveyor 57′ extend near to each other along the longitudinal axis of the screw conveyor housing 59, which runs in one plane or in a straight line with the screw conveyor screw axis 61, so that the grinding mechanism 287 and the screw conveyor 57′ can be operated and/or driven simultaneously by the operating and/or drive device 39. As a result of the actuation and/or drive, the conveyor screw 57′ transports the coffee beans introduced through the inlet 53 from the first container 9′ in the direction of the conveyor screw longitudinal axis 61 to the grinder 287, so that the coffee beans are ground to coffee powder by the grinder 287 and so that the ground Coffee powder leaves the screw conveyor housing 59 through the outlet 67. The screw conveyor 57′ of the device 1′ is designed essentially like the screw conveyor 57 of the device 1, the length of the screw conveyor 57′, 57 differing. In other words, the screw conveyor 57′ is shorter than the screw conveyor 57, so that the length of the screw conveyor 57′ between the opposite ends in the direction of the conveyor screw longitudinal axis 61 is shorter than in the screw conveyor 57.

The screw conveyor housing 59 extends between a first end 71 and an opposite second end 73 along the longitudinal axis of the screw conveyor housing 59, the grinder 287 being arranged adjoining to or near to the first end 71 and extending along the longitudinal axis 297 of the grinder and the longitudinal axis of the screw conveyor housing 59, wherein the screw conveyor 57′ is arranged adjoining or near to the second end 73 and extends along the longitudinal axis 61 of the screw conveyor, with the outlet 67 being arranged adjoining or near to the first end 71 and the inlet 53 of the screw conveyor housing 59 adjoining or near to the second end 73 is arranged. The inlet 53 is arranged above the screw conveyor 57′. The coffee beans can thus enter the screw conveyor housing 59 from the second container 11 solely by the action of gravity and then be conveyed by the screw conveyor 57′ in the direction of the grinder 287. The outlet 67 of the screw conveyor housing 59 is arranged below the grinder 287. The coffee powder ground by the grinder 287 can leave the screw conveyor housing 59 or the screw conveyor and grinder housing 59 through the outlet 67 solely by the action of gravity and be brought into connection with the fluid for preparing coffee.

The grinding mechanism 287 has a first end 301 and an opposite second end 303 along a grinding mechanism longitudinal axis 297. The first end 301 of the grinder 287 is designed as a drive end 289 of the grinder 287. The screw conveyor 57′ has a first end and an opposite second end along the longitudinal axis 61 of the screw conveyor. The first end of the screw conveyor 57′ is designed as a drive end 82 of the screw conveyor 57′

A coupling device 91 extends from the drive end 82 of the screw conveyor 57′ in the direction of the longitudinal axis 61 of the screw conveyor 57′, and a coupling device 291 extends from the drive end 289 of the grinding mechanism 287 along the longitudinal axis 297 of the grinding mechanism. The coupling device 91 of the screw conveyor 57′ is designed to interact in a coupling manner, in particular to intervene, with an actuating and/or drive device 293 of the grinding mechanism 287, which is arranged at the second end of the grinding mechanism 287. The coupling device 91 of the grinder 287 is designed to interact in a coupling manner with the actuating and/or drive device 39 for the dosing and grinding device 29′, in particular to intervene.

In the interconnected state, the coupling device 91 of the screw conveyor 57′ engages with the actuating and/or drive device 293 of the grinder 287 such that the grinder longitudinal axis 297 and the conveyor screw longitudinal axis 61 run in one plane or in a straight line, and in the screw conveyor housing 59 inserted state with the longitudinal axis of the screw conveyor housing 59 extend in one plane or in a straight line. Thus, by actuating or driving the actuating and/or drive device 39 of the device 1′, the grinder 287 and the screw conveyor 57′ can be driven simultaneously via the same shaft.

The coupling device 91 of the screw conveyor 57′ is designed as an essentially cylindrical cavity and/or as a receptacle which extends essentially in the direction of the longitudinal axis 61 of the screw conveyor. Correspondingly, the coupling device 291 of the grinding mechanism 287 is designed as an essentially cylindrical cavity and/or as a receptacle which extends essentially in the direction of the grinding mechanism longitudinal axis 297.

The grinding mechanism 287 has a grinding mechanism core 299 with an essentially conically shaped longitudinal section in the direction of the grinding mechanism longitudinal axis 297. The grinder core 299 extends between the first end 301 and the second end 303 of the grinder 287 in the direction of the grinder longitudinal axis 297. Corresponding to the conically shaped longitudinal section of the grinding mechanism core 299, the periphery of the grinding mechanism core 299, seen transversely to the grinding mechanism longitudinal axis 297, decreases from the first end 301 in the direction of the second end 303.

The grinder 287 has an inner ring 305 adjoining or near to the second end 303. The inner ring 305 extends at least partially around the grinder core 299 from the second end 303 in the direction of the first end 301. The inner ring 305 surrounds the longitudinal axis 297 of the grinder and preferably has a essentially conical longitudinal section along the longitudinal axis 297 of the grinder, the cross-sectional area of the inner ring 305 tapering towards the second end 303.

The inner ring 305 of the grinder 287 seated on the grinder core 299 or on the shaft can be moved by means of an adjusting element 307 along the grinder longitudinal axis 297, in the direction of the first end 301 and/or in the direction of the second end 303 of the grinder 287. The adjusting element 307 is arranged adjoining or near to the first end 301 and surrounds the longitudinal axis 297 of the grinding machine concentrically. By means of the adjusting element 307, the inner ring 305 can be displaceable in the direction of the first end 301 and/or in the direction of the second end 303 of the grinder 287. This enables a degree of grinding to be set in a simple manner.

The grinder 287 can have a spring element (not shown) which is arranged adjoining or near to the inner ring 305 and/or adjoining or near to the second end 303 of the grinder 287 or the grinder core 299. The spring element can be arranged in a recess 309 within the grinder core 299, the recess 309 extending at least partially in the direction of the grinder longitudinal axis 297 and or parallel to the grinder longitudinal axis 297 and in the direction of the first end 301.

The grinder 287 has an outer ring 311. This outer ring 311 has a essentially cylindrical cross section and an inner periphery that is larger than the outer periphery of the inner ring 305. The outer ring 311 is arranged on the inner wall 313 of the screw conveyor housing 59 and is arranged on the inner wall 313 of the screw conveyor housing 59 by means of a holding element 315, for example a hold-down 315. The hold-down device 315 extends between the first open end 71 of the screw conveyor housing 59, adjoining or near to the adjusting element 307, as far as the outer ring 311 along the inner wall 313 of the screw conveyor housing 59. Outer ring 311 can be held in a fixed or stationary position by holding-down device 315.

The hold-down device 315 does not have to adjoin the outer ring 311 at every point on the circumference of the outer ring 311 so that the outer ring 311 is held in a fixed or stationary position. This is not necessary because of the rigidity of the outer ring, which is preferably made of ceramic material or which preferably comprises ceramic material. It is sufficient that the holding-down device 315 is in contact with the outer ring 311 at least two points or contact points, preferably three points or contact points, so that the outer ring 311 can be held in a fixed or stationary position. The three points can preferably be arranged at 120° intervals around the longitudinal axis of the grinder. This enables the hold-down 315 not to block the outlet 67 or the outlet opening 69 of the screw conveyor housing 59, so that the ground coffee powder can exit from the screw conveyor housing 59.

The outer ring 311 is arranged around the inner ring 305 so that the inner ring 305 can rotate within the outer ring 311 due to the drive of the grinding mechanism 287. By adjusting the degree of grinding by means of the adjusting element 307, the position of the inner ring 305 relative to the outer ring 311 (viewed in the direction of the longitudinal axis 61 of the conveyor screw) can be adjustable, so that an intermediate space 317 between the inner ring 305 and the outer ring 311 can be adjusted. Within the intermediate space 317, the coffee beans can be ground to coffee powder at the interfaces of the inner ring 305 and the outer ring 311. The coffee beans conveyed by the screw conveyor 57′ in the direction of the grinder 287 thus reach the space 317 between the inner ring 305 and the outer ring 311 and can be ground into coffee powder due to the rotation of the inner ring 305 within the outer ring 311. The inner ring 305 and the outer ring 311 are adjoining or near to the outlet 67 or to the outlet opening 69 of the screw conveyor housing 59 is arranged. Thus, the coffee powder ground between the inner ring 305 and the outer ring 311 can exit the screw conveyor housing 59 through the outlet 67.

As FIG. 27 shows, the second receiving area 7′ has a rear wall 157, two spaced apart side walls 159 which are oriented at an angle different from 0° or 180°, in particular essentially transversely to the rear wall 157, a lower limitation 161 which is oriented at an angle different from 0° or 180°, in particular transversely to the side walls 159, 159′, and an open top 163 opposite the lower limitation 161, at least one of the side walls 159′ being an inclined side wall 159′ which is oriented at an angle different from 90°, preferably at an angle between 10° and 50°, more preferably at an angle between 10° and 30°, particularly preferably at an angle of 20°, to the lower limitation 161.

The inclined side wall 159′ is connected or connectable to the rear wall 157 and is spaced apart from the lower limitation 161. The lower edge 319 of the inclined side wall 159′ or the edge 319 of the inclined side wall 159′, which points to the lower limitation 161 of the second receiving area 7, can be adjoining or near to a flange 321 which surrounds the through-hole 223 in the lower limitation 161 can be arranged. The inclined side wall 159′ is designed to receive the second container 11 with fluid and to hold it in the inclined position, so that the second container 11 rests or is supported with one of its outer side walls on the inclined side wall 159′. The second container 11 can be held in an inclined position by the inclined side wall 159′, so that the outlet 209 of the second container 11 can open into the through-hole 223 which is surrounded by the flange 321.

The first container 9 with the dosing and grinding device 29 is arranged in a position that is essentially transverse, preferably at an angle of 90° to the lower limitation 161 and above the lower limitation 21 of the first receiving area 5′ and/or above the lower limitation 161 of the second receiving area 7′ and/or arranged above the through-hole 223. Thus, the outlet 67 from the screw conveyor housing 59 and the outlet 209 of the second container 11 can advantageously open together into the through-bore 223. This enables the ground coffee powder and the fluid to be guided through the through-hole 223 in the device 1″ for dosing and grinding coffee powder and/or for preparing coffee, so that the coffee powder and the fluid can be fed to a container, preferably a filter container. However, it is also conceivable that the outlet 67 from the screw conveyor housing 59 and the outlet 209 of the second container each open through spaced through bores 51, 223, as shown in the first exemplary embodiment of the device 1 (see FIGS. 4 and 18).

Adjoining or near to the inclined side wall 159′, three clamping elements 155 are arranged, which run in a plane or clamping element plane parallel to the inclined side wall 159′. The clamping element plane is oriented at an angle different from 90°, preferably at an angle between 10° and 50°, more preferably at an angle between 10° and 30°, particularly preferably at an angle of 20°, to the lower limitation 161. The second container 11 is held between the inclined side wall 159′ and the clamping elements 155, so that one of the side walls of the second container 11 rests or rests on the inclined side wall 159′ and an opposite side wall of the second container 11 rests on the clamping elements 155. Thus, the fluid (in particular the liquid) can be dosed out of the second container 11 by means of the clamping elements 155.

FIG. 30 shows a first embodiment of a preparation device 323, which can preferably be used to prepare a coffee using the cold brew method or type of preparation. The preparation device 323 comprises a filter and/or funnel container 325 or filter container into which the coffee powder and the fluid can be introduced and/or mixed. Furthermore, the preparation device 323 can have a container 327, for example a coffee cup or a coffee pot. The filter and/or funnel container 325 comprises a sieve or a filter 329 which is arranged inside the container 327. A first closing flap 331 for closing a first cavity area 333 and a second closing flap 335 for closing a second cavity area 337 are arranged at the upper open end of the preparation device 323. It is conceivable that the lid can be placed manually on the filter or the jug. Coffee powder, for example, can be introduced into the first cavity area 333 and, for example, fluid can be introduced into the second cavity area 337, in each case from the device 1′ for dosing and grinding coffee beans and/or for preparing coffee. The preparation device 323 further comprises a stirring element or stirrer 339, which is arranged within the container 327 and/or within the sieve or filter 329 when the container 327 is connected to the preparation device 323. It is also conceivable that the preparation device 323 has only one cavity or cavity area and only one closure cap, through which both the coffee or the coffee powder and the fluid are introduced into the filter and/or funnel container 325. It is also conceivable that the mixture is dispensed with so that the device 11 for dosing and grinding coffee beans and/or for preparing coffee only introduces the coffee powder and the fluid in the desired amount into the filter and/or funnel container 325.

FIG. 31 shows a further embodiment of the preparation device 323′, which can preferably be used to prepare a coffee by means of the dripping method or type of preparation. The preparation device 323′ comprises a container 327′ into which the coffee can be filled. A sieve or a filter 329′ is arranged above the container 327′, which includes the coffee powder and into which cooled water or ice water or fluid mixed with ice cubes is fed from a further container or cooling water container 341. The cooling water tank 341 is preferably arranged above the filter 329′. A valve 343 can be arranged between the cooling water tank 341 and the filter 329′, by means of which the cooling water can be introduced drop by drop into the filter 329′. Instead of the cooling water container 341, the second container 11 with fluid can also be provided, which has cooled fluid in its interior. The second container 11 can for example be cooled by the tempering device so that the fluid can be temperature controlled to the temperature suitable for preparing a coffee by means of the dripping method or type of preparation. As an alternative to temperature control by means of the tempering device, when using a reusable second container 11, ice can be packed or introduced into the interior of the second container.

To prepare the coffee, the preparation device 323′ is connected to the filter 329′ in such a way that the cooling water or fluid can be fed drop by drop into the filter 329′ and can be mixed with the coffee powder so that finished coffee is placed in the container 327′ can be performed. The preparation device 323′ can further include a stirring element or stirrer 339 which is arranged within the container 327′ and/or within the sieve or filter 329′ when the container 327′ is connected to the preparation device 323′ (not shown). It is also conceivable that the preparation device 323′ has at least one cavity or cavity area and at least one closure cap, preferably two cavity areas and two closure flaps, through which the coffee or coffee powder and the fluid into the sieve or into the filter 329′ are introduced (not shown).

It is conceivable that instead of the preparation device 323′, 323′, the 3D acoustic waves are used to mix the coffee powder with the fluid. It is also conceivable that the mixture is dispensed with, so that the device 1′ for dosing and grinding coffee beans and/or for preparing coffee only introduces the coffee powder and the fluid in the desired amount into the filter and/or funnel container 325.

FIGS. 32 to 34 show an embodiment of a first container 9″, in which the grinding device 29″ or the grinding mechanism 287″ is arranged in the outlet 113. By operating the grinder 287″, a defined amount of coffee beans can be ground from the first container 9″ and thus a defined amount of coffee or ground coffee can be dosed at the same time.

The grinder is introduced into the outlet 113 of the first container 9″ and is rotatably arranged therein so that the grinder 287″ and the outlet 113 extend around a common longitudinal axis or around the longitudinal axis 297 of the grinder. In this way, the coffee beans inside the first container 9″ can be guided gravimetrically towards the outlet 113 and ground.

The grinder 293″ is operated and/or driven by an actuation and/or drive device 293″. By actuating and/or driving the coffee beans from the first container 9″ can be ground by the grinder 287″ so that the ground coffee powder can leave the container 9″ or the grinder 287″ through the outlet 113 of the first container 9″.

The grinder 287″ comprises a first end 301 and an opposite second end 303 along the longitudinal axis 297 of the grinder. The first end 301 protrudes from the outlet 113 of the first container 9″ and is therefore arranged outside the first container 9″. The second end 303 is to be arranged inside the first container 9″. Because the first end 301 of the grinder 287″ protrudes from the outlet 113, the first end 301 can be designed as a drive end 301 of the grinder 287″.

A coupling device 291″ is arranged at the drive end 301 of the grinder 287″. The coupling device comprises a gear transmission with a gear 294, by means of which the grinding device 29″ or the grinder 287″ can be driven.

The grinding mechanism 287″ comprises a grinding mechanism core 299″ with an essentially conically shaped longitudinal section in the direction of the grinding mechanism longitudinal axis 297. The grinder core 299″ extends between the first end 301 and the second end 303 of the grinder 299″ in the direction of the longitudinal axis 297 of the grinder.

The grinder 287″ has an inner ring 305″ adjoining or near to the second end 303. The inner ring 305″ extends at least partially around the grinder core 299″ from the second end 303 in the direction of the first end 301.

The grinder 287″ further comprises an outer ring 311″. The outer ring 311″ adjoins the inner wall of the outlet 113 of the first container 9″ and is arranged around the inner ring 305″ so that the inner ring 305″ can rotate within the outer ring 311″ due to the drive of the grinder 287″. By adjusting the degree of grinding, for example by means of an adjusting element, the position of the inner ring 305″ relative to the outer ring 311″ (viewed in the direction of the longitudinal axis 297 of the grinder) can be adjusted so that a gap between the inner ring 305″ and the outer ring 311″ can be adjusted. Within the space 311″, the coffee beans can be ground to coffee powder at the limitation surfaces of the inner ring 305″ and the outer ring 311″.

The actuation and/or drive device 293″ comprises a motor 318, the motor 318 being designed to drive the grinder 287″. The motor 318 comprises a gear 293 which is arranged in relation to the gear 294 of the coupling device 291″ so that the gear 293 of the motor 318 comes into contact with the gear 294 of the coupling device 291″ of the grinder 287″ and the grinding device 30 or the grinder 287″ Is driven.

FIGS. 35 and 36 show a plurality of preparation devices 345 for different types of preparation, for example filter coffee 347, cold brew 349, cold drip 351, espresso 353, and Karlsbader 355. The preparation devices 345 (or preparation units) have at least one ring 357. Some of the rings 357 have at least one notch 359. This enables the device to determine the number of rings 357 and/or the number of notches 359 or the presence of a notch 359 via corresponding sensor elements (not shown) and thus to determine the respective preparation device 345 and then the corresponding type of preparation, e.g. filter coffee, cold brew, cold drip, espresso, Karlsbader, etc., can carry out.

The system, as previously described, comprises a device 1′ for dosing and grinding coffee beans and/or for preparing coffee, a first container 9′ for receiving and dosing coffee beans and a second container 11 for receiving and dosing fluid (in particular liquid), so that the coffee can be prepared using this system. For this purpose, the first container 9′ is designed to receive and dose coffee and to receive and interact with the device 1′. The second container 11 is designed for receiving and dosing fluid and for receiving and interacting with the device 1′. The device 1′ comprises the first receiving area 5′, which is designed to receive the first container 9′ and to receive and drive the dosing and grinding device 29′ that can be connected to the container 9′. As the dosing and grinding device 29′ is driven by the actuating and/or drive device 39, which is arranged in the dosing and grinding device receiving area 27′ within the first receiving area 5′, the coffee beans can be moved from the first container 9′ into the Dosing and grinding device 29′ are introduced and correctly dosed by this and ground to coffee powder. The device 1′ also comprises the second receiving area 7′, which is designed to receive the second container 11. The fluid can be correctly dosed by means of the clamping elements 155 which are arranged in the second receiving area 7′. With the device 1′, the ground coffee powder from the dosing and grinding device 29′ and the fluid from the second container 11 can be fed to a preparation device and introduced into a container, in particular into a filter container, in the correct mixing ratio. Thus, the device 1′ enables a simplified, correct and safe preparation of coffee.

### LIST OF REFERENCE SYMBOLS

1, 1′ device
3 housing
5, 5′ first receiving area
7, 7′ second receiving area
9, 9′, 9″ first container
11 second container
13 rear wall
15 side wall
17 side wall
19 upper limitation
21 lower limitation
23 open front
25 container receiving area
27, 27′ dosing device receiving area, dosing and grinder receiving area
29, 29′, 29″ dosing device, dosing and grinding device
31 guide element
33 guide element
35 essentially parallel area of the guide elements
37 inclined area of the guide elements
39 actuation and/or drive device
41 coupling element or drive shaft
43 holder for a dosing device
45 longitudinal axis
47 first horizontal surface section
49 second horizontal surface section
51 receptacle outlet opening
53 plurality of ribs
55 pairs of ribs
57, 57′ screw conveyor
59 screw conveyor housing
61 screw conveyor longitudinal axis
63 inlet of the screw conveyor housing
65 inlet opening
67 outlet of the screw conveyor housing
69 outlet opening
71 first end of the screw conveyor housing
73 second end of the screw conveyor housing
75 flange
77 peripheral wall
78 first peripheral wall central longitudinal axis
79 first contact surface
80 second peripheral wall central longitudinal axis
81 second contact surface
82 drive end of the screw conveyor
83 outer wall of the screw conveyor housing
85 plurality of ribs
87 first pair of limiting ribs
89 second pair of limiting ribs
91 coupling device
93 cylindrical cavity
95 inner wall of the cylindrical cavity
96 outer wall of the coupling element
97 at least one material elevation
99 at least one material recess
101 helically wound flights
103 screw thread
105 insertion element or removal element
107 edges of the guide elements
109 bottom
111 housing of the first container
112 interior space of the first container
113 outlet
115 inlet opening of the first container
117 outlet opening
119 closure element
121 tab
123 inner opening
125 tapered section
127 first essentially symmetrical section
129 second essentially symmetrical section
131 first side edge of the first essentially symmetrical section
133 second side edge of the first essentially symmetrical section
135 first side edge of the tapered section
137 second side edge of the tapered section
139 first side edge of the second essentially symmetrical section
141 second side edge of the second essentially symmetrical section
143 plate
145 cover
147 flat surface
149 connecting element
151 adhesive strip
153 clip
155 clamping elements of the side walls of the second receiving area
157 rear wall of the second receiving area
159, 159′ side walls
161 lower limitation
163 open top
165 front
167 first, lower pair of clamping elements
169 second, upper pair of clamping elements
171 third, middle pair of clamping elements
D1 first distance
D2 second distance
D3 third distance
173 first side wall of the second container
175 second side wall of the second container
177 first clamping element
179 second clamping element
181 first clamping element surface
183 second clamping element surface
185 clamping element longitudinal axis
186 third clamping element surface
187 first end of the clamping element surfaces
189 second end of the clamping element surfaces
B width of the clamping element surfaces
191 connecting plate
192 through hole
193 first side edge of the first clamping element surface
195 first side edge of the second clamping element surface
197 clamping element edge
199 inner cavity
201 open side of the cavity
203 housing of the second container
205 interior space
207 inlet of the second container
209 outlet of the second container
211 outlet of the fluid reservoir
213 fluid reservoir
215 inlet opening of the second container
217 longitudinal container axis
219 outlet opening of the second container
221 lower area of the second receiving area
223 through hole in the lower limitation
225 upper area of the second receiving area
227 first end of the outlet of the second container
229 second end of the outlet of the second container
231 longitudinal outlet axis
233 inlet end
235 outlet end
241 tapered section of the second container
243 essentially symmetrical section of the second container
245 essentially horizontal plate
247 through hole of the plate
249 first flange of the plate
251 first peripheral wall of the plate
253 first side of the plate
255 outlet opening of the fluid reservoir
257 first thread
259 second thread
261 peripheral wall of the outlet of the fluid reservoir
262 second side of the plate
263 second flange of the plate
265 second peripheral wall of the plate
267 flange center longitudinal axis
269 fluid reservoir central longitudinal axis
271 housing fluid reservoir
273 top of the fluid reservoir
275 bottom of the fluid reservoir
277 at least a first magnet
279 outer wall of the tapered section
281 at least a second magnet
283 inner wall of the through hole
284 combination container or combination bag
285 positioning and holding device
287, 287″ grinder
289 drive end of the grinder
291, 291″ coupling device of the grinder
293, 293″ actuating and/or drive device
294 gear or pinion
295 gear or pinion
297 longitudinal axis of the grinder
299, 299″ grinder core
301 first end grinder
303 second end grinder
305, 305″ inner ring
307 adjustment element
309 recess
311, 311″ outer ring
313 inner wall of the screw conveyor housing
315 holding element/hold-down
317, 317″ space
318 motor
319 lower edge of the inclined side wall
321 flange surrounding the through-hole
323, 323′ preparation device
325 filter and/or funnel container
327 container
329 sieve or filter
331 first flap
333 first cavity area
335 second flap
337 second cavity area
339 stirrer or stirrer element
341 cooling water tank
343 valve
345 preparation device
347 filter coffee—preparation device
349 cold brew—preparation device
351 cold drip preparation device
353 espresso preparation device
355 Karlsbader—preparation device
357 at least one ring
359 at least one notch

1. Device (1; 1′) for dosing and/or preparing a medium to be prepared, in particular baby food, in particular baby milk or baby mash, coffee and/or tea, the device comprising:
a housing (3) with a first receiving area (5; 5′) and a second receiving area (7; 7′), the first receiving area (5; 5′) for receiving a first container (9; 9′; 9″) is designed for a first component of the medium to be prepared and wherein the second receiving area (7; 7′) is designed to receive a second container (11) for a fluid,
a tempering device for tempering the fluid,
a dosing device (29; 29′; 29″) for dosing the first component,
wherein the first receiving area (5; 5′) comprises a dosing device receiving area (27; 27′) for receiving the dosing device (29; 29′; 29″),
an actuating and/or drive device (39) for the dosing device (29; 29′; 29″) is arranged in the dosing device receiving area (27; 27′),
the dosing device (29; 29′) is connected to the first container (9; 9′; 9″) and the first container (9; 9′; 9″) and the dosing device (29; 29′; 29″) are interchangeable and are designed as disposable articles,
the dosing device (29; 29′) comprises a screw conveyor (57; 57′) and a screw conveyor housing (59), the screw conveyor (57; 57′), preferably in its full length, is inserted in the screw conveyor housing (59) and rotatably arranged therein, and
the screw conveyor housing (59) has an inlet (63) with an inlet opening (65) and an outlet (67) with an outlet opening (69).

2-3. (canceled)

4. Device according to claim 1, wherein the second container (11) is connectable to a fluid reservoir (213), wherein the second container (11) and/or the fluid reservoir (213) are exchangeable and are designed as disposable article, and/or
the dosing device (29′; 29″) has a grinding device (29′; 29″), the grinding device (29′; 29″) preferably being designed for grinding and/or dosing and grinding, and/or
the screw conveyor (57; 57′) and the screw conveyor housing (59) extend around a common screw conveyor longitudinal axis (61);
the inlet (63) and the outlet (67) are furthermore preferably arranged on opposite sides, viewed transversely to the longitudinal axis (61) of the conveyor screw, in the screw conveyor housing (59); and
the screw conveyor housing (59) preferably extending between a first end (71) and an opposite second end (73) along the conveyor screw longitudinal axis (61), the outlet (67) preferably being arranged adjoining or near to the first end (71) and wherein the inlet (63) is disposed adjoining or near to the second end (73).

5-6. (canceled)

7. Device according to claim 1, wherein from a drive end (82) of the screw conveyor (57; 57′) a coupling device (91) extends in the direction of the longitudinal axis of the screw conveyor (57; 57′), the coupling device (91) being designed to interact in a coupling manner with, in particular to engage the actuation and/or drive device (39), and/or
the screw conveyor housing (59) has an outer wall (83) with a plurality of ribs (85),
the ribs (85) preferably extend in the axial direction at least partially between the first end (71) and the second end (73), and/or
the ribs (85) extend away from the outer wall (83) essentially in the radial direction,
preferably two of the ribs (85) limit the outlet opening (69) on opposite sides in the peripheral direction of the outer wall (83), and
preferably two further ribs (85) delimiting the outlet opening (69) on opposite sides in the axial direction of the outer wall (83).

8. (canceled)

9. Device according to claim 1, wherein the first receiving area (5) comprises a container receiving area (25) for receiving the first container (9),
wherein the container receiving area (25) is preferably arranged above the dosing device receiving area (27; 27′); and/or
one or more side walls (15, 17) of the container receiving area (25) comprises a plurality of ribs (53) which extend away from the one or more side walls (15, 17); and/or
a first guide element (31) and a second guide element (33) are arranged between the container receiving area (25) and the dosing device receiving area (27; 27′),
the guide elements (31, 33) extend from the open front side (23) to the rear wall (13); and/or
the guide elements (31, 33) extend away from the side walls (15, 17).

10-11. (canceled)

12. Device according to claim 1, wherein the device comprises a preparation device (323; 323′) for preparing the medium to be prepared from the first component and the fluid,
the device is preferably designed to determine the presence and/or the type of preparation device (323; 323′); and/or
the dosing device (29; 29′) comprises a closure or flap element,
the closure or flap element is designed to be opened automatically or manually, and
the closure or flap element is preferably designed to close the dosing device (29; 29′) and/or the first container (9; 9′) airtight.

13. (canceled)

14. Container (9; 9′) for receiving and dosing a component for the preparation of a medium, in particular baby food, in particular baby milk or baby mash, coffee and/or tea, the container (9; 9′) comprising:
a housing (111) having an interior space (112) for receiving the component; and
an outlet (113) in fluid communication with the interior space (112),
wherein the outlet (113) connectable to an inlet (63) of a dosing device (29),
the dosing device (29; 29′) has an outlet (67), so that by actuating the dosing device (29) a dosage of the component is dispensed through the outlet (67);
the dosing device (29; 29′) is connected or connectable to the container,
the container (9; 9′) and/or the dosing device (29; 29′) are exchangeable and are designed as disposable article,
the dosing device (29; 29′) comprises a screw conveyor (57; 57′) and a screw conveyor housing (59), and
the screw conveyor (57; 57′), preferably in its full length, can be inserted rotatably into the screw conveyor housing (59).

15. Container according to claim 14, wherein the container (9; 9′) can be supplied pre-filled with the component and/or wherein the dosing device (29′) has a grinding device (29′), the grinding device (29′) preferably is designed for dosing and grinding.

16. Container (9; 9′) according to claim 14,
the screw conveyor (57; 57′) and the screw conveyor housing (59) extend along a common screw conveyor longitudinal axis (61), and
the inlet of the dosing device (29; 29′) is arranged in or on the screw conveyor housing (59);
the outlet (113) of the container (9; 9′) is firmly connected, preferably screwed or glued, to the inlet (63) in the screw conveyor housing (59); and/or
the screw conveyor housing (59) is integrated into the container (9; 9′).

17-19. (canceled)

20. Device according to claim 1, the second receiving area (7) comprising a rear wall (157), two spaced-apart side walls (159; 159′) which are oriented transversely to the rear wall (157), a lower limitation (161), which is oriented transversely to the side walls (159; 159′), and an open upper side (163) opposite the lower limitation (161),
wherein the second receiving region (7) for receiving the second container (11) is formed between the side walls (159; 159′); and/or
adjoining or near the side walls (159; 159′) a plurality of clamping elements (155) is arranged which extend at least partially between a front side (165) opposite the rear wall (157) and the rear wall (157) of the second receiving area (7).

21-26. (canceled)

27. Device according to claim 20, wherein the tempering device is arranged in contact with the second container (11),
the tempering device is preferably arranged in an area adjoining or near the lower limitation (161) of the second receiving area (7);
the tempering device is arranged in an area adjoining or near one of the clamping elements (155) which is closest to the lower limitation (161);
preferably at least one of the clamping elements (155) is replaced by the tempering device for tempering the fluid to be dosed by means of the clamping elements (155); and/or
the at least one of the clamping elements (155) is adjustable or height-adjustable in the clamping element plane.

28-32. (canceled)

33. Device according to claim 1, wherein by means of application software, for example a mobile app, the filling level of the first component in the first container (9; 9′) and/or the filling level of the fluid in the second container (11; 11′) is automatically indicated, for example by a signal tone or a signal light, and/or
the first container (9; 9′), for example after emptying the first component or after reaching a certain level, and/or the second container (11; 11′), for example after Emptying of the fluid or after reaching a certain level, can be ordered automatically on the Internet.

34. (canceled)

35. Device according to claim 1, wherein the first container (9; 9′) and/or the second container (11; 11′) and/or the dosing device (29; 29′) and/or the clamping elements (155) comprise a bioplastic or bioplastics or a bio-based plastic, preferably stone paper and/or wood.

36. Device according to claim 1, further comprising a positioning and holding device (285) which is designed to position and hold the second container (11; 11′) in the second receiving area (7, 7′).

37. Container (11) for receiving and dosing fluid for the preparation of a medium to be prepared, in particular baby food, in particular baby milk or baby mash, coffee and/or tea; the container (11) comprising:
a housing (203) having an interior space (205) for receiving fluid, and
an outlet (209) in fluid communication with the interior space (205),
a dosage of the fluid for preparing the medium to be prepared can be delivered through the outlet (209) of the container (11), and
the container (11) is exchangeable and designed as a disposable article, and
the container (11) comprises a positioning and holding device (285) which is designed to position and hold the container (11) in the second receiving area of a device for dosing and/or preparing a medium to be prepared.

38. Container (11) according to claim 37, wherein the container (11) comprises an inlet (207) in fluid communication with the interior space (205) and wherein the inlet (207) is connectable to an outlet (211) of a fluid reservoir (213), and/or the container (11) is deliverable pre-filled with the fluid, and/or
the inlet (207) of the container (11) is firmly connected, preferably screwed or glued, to the outlet (211) of the fluid reservoir (213), and/or
the fluid reservoir (213) is integrated into the container (11), and/or
the inlet (207) of the container (11) comprises an inlet opening (215) which is preferably essentially opposite to the outlet (209) of the container (11) and/or essentially is arranged opposite an outlet opening (219) in the outlet (209) of the container (11).

39-41. (canceled)

42. Container (11) according to claim 38, wherein the container (11) comprises an essentially horizontal plate (245) which is arranged adjoining or near the inlet (207) of the container, wherein the horizontal plate (245) preferably is integrated into the fluid reservoir (213),
preferably, the plate (245) can be connected to the container (11) or wherein the plate (245) is firmly connected to the container (11) or wherein the plate (245) is integrated into the container (11), and/or
the first flange (249) is designed to connect the plate (245) to the outlet (211) and/or to an outlet opening (255) of the fluid reservoir (213), and/or
the container (11) further comprises at least one magnet (277), and
preferably, the at least one magnet (277) is arranged on an outer wall (279) of a tapering section (241) of the container (11).

43-48. (canceled)

49. Computer-implemented method for controlling or regulating a device for dosing and/or preparing a medium to be prepared, in particular baby food, in particular baby milk or baby food, coffee and/or tea, according to claim 1, the method comprising:
Dosing the first component from the first container (9; 9′) by means of the dosing device (29; 29′),
Dosing of the fluid from the second container (11, 11′) by means of a further dosing device.

50-51. (canceled)

52. System comprising a device (1; 1′) for preparing a medium to be prepared, in particular baby food, in particular baby milk or baby mash, coffee and/or tea according to claim 1, a first container (9; 9′) for receiving and dosing a component for preparing a medium, and/or a second container (11) for receiving and dosing of fluid for the preparation of a medium to be prepared, in particular baby food, in particular baby milk or baby mash, coffee and/or tea.

53. Device according to claim 1, wherein the device comprises three clamping elements (155),
the tempering element is arranged between a first, lower clamping element and a third, middle clamping element, or
the tempering element is arranged between a first, lower clamping element and a second, upper clamping element, wherein preferably, the tempering element extends between the first, lower clamping element and the third, middle clamping element or the second, upper clamping element, and/or
at least one of the side walls of the second receiving area is an inclined side wall which is inclined at an angle different from 90°,
the inclined side wall with the heating element serve as a counter surface to one of the clamping elements, preferably to the lower clamping element, so that the second container can be arranged between the lower clamping element and the heating element and/or the inclined side wall, or to one of the other two clamping elements.

54. Device according to claim 53, wherein the device comprises more than one tempering element, preferably two tempering elements,
each of the tempering elements is arranged adjoining or near to the inclined side wall, and/or
the first, lower clamping element is configured to press a lowest point of the second container in the state received in the second receiving area, preferably an area adjoining to or near to an outlet opening of the second container, against the tempering element, and/or
the three clamping elements are configured to assume at least one position, and
the position of the clamping elements can be changed or displaced relative to the side walls or relative to the inclined side wall and/or relative to the lower boundary of the second receiving area.
